(19) United States
(12) Patent Application Publication
(10) Pub. No.: US 2016/0083791 A1
(43) Pub. Date: Mar. 24, 2016

(54) SYSTEM AND METHOD FOR DETECTING ABNORMALITIES IN CERVICAL CELLS

(71) Applicant: PATHADVANTAGE ASSOCIATED, Dallas, TX (US)

(72) Inventors: Alexandra Jean GILLESPIE, Dallas, TX (US); Richard Thornton HOPLEY, Allen, TX (US); Jennifer Rebecca TINKLER, Dallas, TX (US)

(73) Assignee: PATHADVANTAGE ASSOCIATED

(21) Appl. No.: 14/489,726

(22) Filed: Sep. 18, 2014

(51) Int. Cl. C12Q 1/68

(52) U.S. Cl. CPC C12Q 1/6881

(57) ABSTRACT

The present disclosure is directed to a method for identifying an abnormal sample of cells by (a) hybridizing a set of chromosomal probes to the sample, wherein the set comprises probes to 3q, 5p, CEP7, and 20; (b) evaluating cells of the sample to detect and quantify the presence of each probe in the set; (c) categorizing the evaluated cells of the sample as normal or abnormal, wherein the normal cells contain exactly two copies of each probe in the set and the abnormal cells do not contain exactly two copies of each probe in the set; (d) calculating the percentage of the abnormal cells in the evaluated cells of the sample; and (e) identifying the sample of cells as abnormal if the percentage of abnormal cells in the evaluated cells is greater than or equal to a predetermined cut-off threshold value.

### FIELD OF THE INVENTION

The present disclosure relates generally to a system and method for detecting abnormalities in human cervical and vaginal cells.

### BACKGROUND

Worldwide, cervical cancer is both the fourth most common cause of cancer and deaths from cancer in women (http://en.wikipedia.org/wiki/Cervical_cancer; accessed Sep. 18, 2014). In 2012, it was estimated that there were 528,000 cases of cervical cancer, and 266,000 deaths. It is the second most common cause of female specific cancer after breast cancer accounting for around 8% of both total cancer cases and total cancer deaths in women. Approximately 80% of cervical cancers occur in developing countries.

It is estimated that human papillomavirus (HPV) is associated with 500,000 new cases of cervical cancer and 250,000 cervical cancer deaths worldwide each year. Within the US, it was estimated for 2008 that 11,070 new cases would be diagnosed, and about 3,870 women would die of their disease (Jemal A, Siegel R, Ward E, et al., CA Cancer J. Clin. (2008), 58(2):71-96). The disease usually presents in several premalignant stages ranging from mild dysplasia (cervical intraepithelial neoplasia grade 1 (CIN1) to more severe degrees of neoplasia and microinvasive lesions (CIN2 or CIN3), to invasive cancer. Classification of the disease according to this CIN System forms the basis of diagnosis and treatment approaches including therapeutic options and secondary preventive measures. Importantly, CIN1 lesions can regress spontaneously with the risk of progression to severe dysplasia being 1% per year.

Historically, the primary screening program for this disease has relied upon the cytologic appearance of abnormal cells in the transformation zone of the cervix (Pap test). A single cytologic examination is relatively insensitive, poorly reproducible and frequently yields equivocal results. In the United States, about 55 million Pap smears are performed each year, and of these approximately 5% (2,750,000 smears per year) are diagnosed as containing atypical squamous cells of undetermined significance (ASCUS) and require follow-up testing, and 5-10% of ASCUS patients have undetected cancer. It is known that about 39% of women with high grade disease (CIN2/CIN3 or frank cancer) will actually present as ASCUS. Thus, considering the 2,750,000 smears diagnosed as ASCUS each year, just under 10% have underlying CIN3 or cancer. Current guidelines for patients include follow-up Pap testing, testing for high-risk human papilloma virus (HR HPV, or HPV) and/or colposcopy.

Infection with HPV is associated with cervical cancer and many patients are tested for HPV after an ASCUS Pap test result. The strength of sensitive HPV testing is that it provides extremely high negative predictive value; women who test negative are at low risk for developing cervical cancer. However, the positive predictive value of HPV testing is limited since only a small fraction of HPV positive early lesions progress to high-grade dysplasia and cancer. Thus, HPV detection, even in combination with cytomorphological evaluation, is a test with poor specificity.

In addition, approximately 3% of Pap tests are diagnosed with low-grade squamous intraepithelial lesions (LSIL). Current guidelines for these patients recommend additional monitoring and/or colposcopy. Clinical studies show the majority of these patients are HPV+.

There is significant risk for an ASCUS/HPV+ or LSIL patient to progress to more severe cervical disease and require surgical treatment in the two years following the initial test. The identification of these patients that will progress is impossible based on morphology and HPV infection. Genetic alterations have been identified in the early development of cervical cancer that can predict the patient's risk of disease progression. These aberrations include changes in DNA content (e.g. ploidy) and the amplification of portions of chromosomal DNA.

To date, gains of 3q, 5p, and 20q have been the most commonly and consistently observed genomic copy number alterations in cervical cancer, which are also found in the other anogenital cancers. However, variability occurs in the reported frequencies of these gains, which is mostly attributed to differing cut-offs values for the presence/absence of the abnormality. For example, Heselmeyer-Haddad et al. developed algorithms to permit classification of HSIL specimens, that were dependent on the particular cut-off used. Overall though, there is evidence that these abnormalities are present in precancerous lesions and may have roles in cervical carcinogenesis. In a follow-up study by Heselmeyer-Haddad et al. (Am. J. Pathol. (2005), 166(4): 1229-38) of precancerous used pap smears (total of 59), gain of 3q (TERC) was associated with progression of CIN1/CIN2 lesions to more dysplastic lesions, while none of the CIN1/CIN2 cases that regressed showed the abnormality, using cut-offs re-established for used cervical smears. The sensitivity of prediction of progression was 100% and specificity was 70%. Additionally, gain of 3q was found in 33% of cytologically normal smears from women who at later times displayed CIN3 or cervical cancer. Thus, at least for gain of 3q, there is preliminary evidence that this genomic lesion may serve as biomarker of disease progression.

HPV infection is thought to lead to chromosomal instability (resulting in the abnormalities described above) and ultimately transformation. To this end, associations between HPV infection and particular genomic abnormalities have been assessed. A recent FISH study combined detection of the HPV genome with the detection of 3q and 8q gain in 235 residual liquid cervical specimens (Sokolova I, Algeciras-Schimnich A, Song M, et al, J. Mol. Diagn. (2007), 9(5):604-11). This study showed an increase in the number of “double positive” cells (positive for both HPV and 3q and/or 8q gain) with increasing degree of dysplasia and using a cut-off of four cells, that 80% of CIN2/3 cases were “double positive”.

Other studies have shown an amplification in both a portion of chromosome 3, specifically locus 3q26, that includes a gene TERC that encodes a subunit of the telomerase protein and a portion of chromosome 5, specifically 5p15, that includes a gene, TERT, that encodes another subunit of the telomerase protein, both of which are linked to cell immortality. Studies have demonstrated multicolor fluorescent DNA probes can detect abnormalities in both ploidy, and 3q and 5p copy number by fluorescence in situ hybridization (FISH) with greater sensitivity and specificity than other methods.

The implementation of cervical cancer screening programs has greatly reduced disease incidence and mortality in industrialized countries. However, a single cytological evaluation remains relatively insensitive, hence the need for frequent follow-up investigations. This is attributable to sampling or interpretation errors, and to the fact that some early lesions may not have acquired recognizable phenotypic alterations.

Invasive cervical carcinomas develop through increasing stages of cervical dysplasia, to cervical intraepithelial neoplasia (CIN) 1, CIN2, CIN3 and to carcinoma in situ, which is considered a bona fide precancerous lesion that requires surgical intervention. However, only about 15% of all low-grade dysplastic lesions follow this path of linear progression. Pap and HPV tests are indirect methods for determining the presence of cervical dysplasia or cancer. Therefore, there is a continuing unmet need for identifying the presence of dysplasia or cancer and monitoring disease progression.

### BRIEF SUMMARY OF THE INVENTION

The present disclosure is directed to a system and method for detecting abnormalities in in a cell sample.

In certain embodiments, the present disclosure is directed to a method for identifying an abnormal sample of cells comprising the steps of: (a) hybridizing a set of chromosomal probes to the sample, wherein the set comprises probes to 3q26, 5p15, CEP7, and 20q13; (b) evaluating cells of the sample to detect and quantify the presence of each probe in the set; (c) categorizing the evaluated cells of the sample as normal or abnormal, wherein the normal cells contain exactly two copies of each probe in the set and the abnormal cells do not contain exactly two copies of each probe in the set; (d) calculating the percentage of the abnormal cells in the evaluated cells of the sample; and (e) identifying the sample of cells as abnormal if the percentage of abnormal cells in the evaluated cells is greater than or equal to a predetermined cut-off threshold value.

In specific embodiments, the disclosure relates to a system and method for detecting abnormalities in cervical, vaginal, or anal cells. In further embodiments, the present disclosure is directed to detecting abnormal cells that are categorized as cells having a single gain, cells having multiple gains, tetra-ploid cells, and combinations thereof. Also, the present disclosure provides for a system and method for detecting abnormalities in cervical cells based on threshold values obtained in validation studies.

The present disclosure also relates to manual and automated systems and methods for detecting abnormalities in cervical cells.

### BRIEF DESCRIPTION OF THE SEVERAL VIEWS OF THE DRAWING

FIG. 1a. Microscopic image of a normal cell showing the combined images of the red filter (3q26), green filter (5p15), aqua filter (CEP7), and gold filter (20q13). The image shows exactly two (2) copies of each probe present in the cell.

FIG. 1b. Image depicted in FIG. 1a with the colors inverted to show the probes as dark spots on the lighter cell background.

FIG. 2a. Microscopic image of an abnormal cell showing the combined images of the red filter (3q26), green filter (5p15), aqua filter (CEP7), and gold filter (20q13). The image shows multiple copies of each probe present in the cell.

FIG. 2b. Image depicted in FIG. 2a with the colors inverted to show the probes as dark spots on the lighter cell background.

FIG. 3. Exemplary HPV-4C DNA Damage Test Report for reporting negative results from samples tested by methods disclosed herein.

FIG. 4. Exemplary HPV-4C DNA Damage Test Report for reporting negative results from samples tested by methods disclosed herein.

### DETAILED DESCRIPTION

The present disclosure is directed to a system and method for screening and detecting a variety of abnormalities and conditions that may be present in a cell sample.

As used herein, the term “sample” relates to any liquid or solid sample collected from a subject to be analyzed. In some embodiments, the sample is liquefied at the time of assaying. In other embodiments, the sample is a suspension of single cells disintegrated from a tissue biopsy such as a tumor biopsy. In other embodiments, the sample is a tissue sample, for example, a tissue section mounted on a slide. In other embodiments, the sample comprises genomic DNA, mRNA or rRNA. The sample to be analyzed can be collected from any kind of animal subject to be evaluated. In some embodiments, the animal subject is a mammal, including a human being, a pet animal, and a zoo animal. In other embodiments, the sample is derived from any source such as body fluids. Preferably, this source is selected from the group consisting of milk, semen, blood, serum, plasma, saliva, faeces, urine, sweat, ocular lens fluid, cerebral spinal fluid, cerebrospinal fluid, ascites fluid, mucous fluid, synovial fluid, peritoneal fluid, vaginal discharge, vaginal secretion, cervical discharge, cervical or vaginal swab material or pleural, amniotic fluid and other secreted fluids, substances, cultured cells, and tissue biopsies. One embodiment relates to a method in which the sample or biological sample is selected from the group consisting of blood, vaginal washings, cervical washings, cultured cells, tissue biopsies such as cervical biopsies, and follicular fluid. Another embodiment relates to a method in which the biological sample is selected from the group consisting of blood, plasma and serum. The sample taken may be dried for transport and future analysis. Thus, the present disclosure includes the analysis of both liquid and dried samples. In some embodiments, the sample is pre-treated prior to analysis. Pre-treatment relates to any kind of handling of the sample before it has been applied to the disclosed system or method. Pre-treatment procedures includes separation, filtration, dilution, distillation, concentration, inactivation of interfering compounds, centrifugation, heating, fixation, addition of reagents, or chemical treatment.

As used herein, the terms “biopsy” and “biopsy specimen” are intended to mean a biological sample of tissue, cells, or liquid taken from the human body.

The term “specimen” generally refers to a sample used for medical testing.

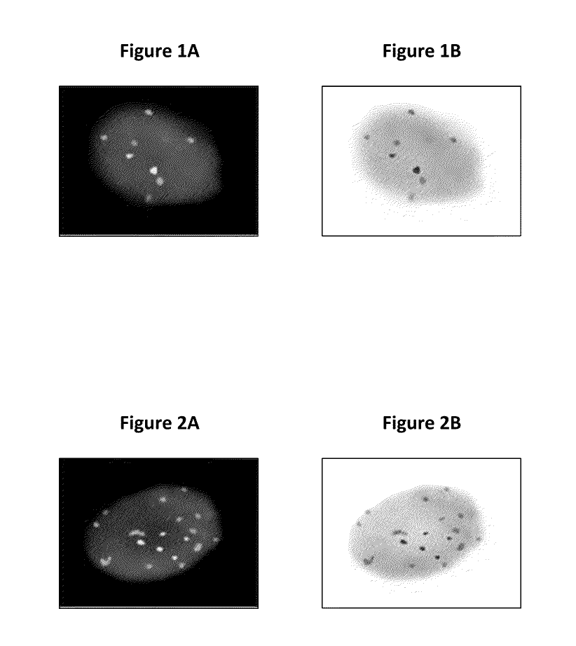

The term “abnormal cell” as used herein, refers to any cell that appears atypical under a microscope or that functions differently than it should compared to a normal cell. Abnormal cells include benign, infected, inflamed, dysplastic, precancerous, and true cancerous cells. In some embodiments, cells are classified as “normal” or “abnormal” based on the number of chromosomes or chromosomal regions detected in the cells. In this embodiment, a “normal” human somatic cell is one that contains 46 chromosomes, representing two complete haploid sets, which make up 23 homologous chromosome pairs (FIGS. 1a and 1b). Accordingly, an “abnormal” human somatic cell is characterized as one that contains more or less than 46 chromosomes (e.g., FIGS. 2a and 2b). In this embodiment, abnormal cells include cells that contain extra or missing chromosome(s). Thus, abnormal cells include cells that are monoploid (1 set), diploid (2 sets), triploid (3 sets), tetraploid (4 sets), pentaploid (5 sets), hexaploid (6 sets), heptaploid/septaploid (7 sets), etc. The generic term polyploid is frequently used to describe cells with three or more sets of chromosomes (triploid or higher).

The term “abnormal sample” as used herein, refers to a sample that has been analyzed and determined to contain one or more abnormalities as assessed by certain criteria. In some embodiments, an abnormal sample contains one or more abnormal cells, as defined herein. In particular embodiments, a sample of cells is evaluated by the disclosed methods and is considered abnormal if the sample contains more than a predetermined cut-off (threshold) value of abnormalities.

As used herein, the terms “cancer” and “cancerous” are intended to mean the physiological condition in mammals that is typically characterized by unregulated cell growth. Examples of cancer include any cancer associated with HPV, including, for example, cancers of the cervix, anus, vulva, vagina, penis, oropharynx, and pharynx.

As used herein, the terms “precancer” and “precancerous” are intended to mean the physiological condition in mammals that is typically characterized by unregulated cell growth that will progress to cancer. Examples of precancer include any precancer associated with HPV including, for example, precancers of the cervix, anus, vulva, vagina, penis, oropharynx, and pharynx.

As used herein, “cervical cell disorder,” “cervical disorder,” or “cervical disease” means any of the following: cervical carcinogenesis, Human Papilloma Virus (HPV) positive, Atypical Squamous Cells of Undetermined Significance (ASCUS), Low-grade Squamous Intraepithelial Lesion (LSIL), Atypical Squamous Cells-cannot exclude high-grade squamous intraepithelial lesion (ASC-H), Atypical Glandular Cells of Undetermined Significance (AGUS), High-grade Squamous Intraepithelial Lesion (HSIL), cervical dysplasia, pre-cancer, pre-malignant legion, cervical cancer, cervical adenocarcinoma, cervical squamous cell carcinoma, cervical intraepithelial neoplasia 1 (CIN1), cervical intraepithelial neoplasia (CIN2), cervical intraepithelial neoplasia 3 (CIN3), carcinoma in situ, invasive cervical carcinoma, and cytological or genetic abnormality of the cell. Also, “disease,” “cell disorder,” or “disorder” as used herein includes but is not limited to any cytological or genetic abnormality of the cell.

The term “cervical cancer” as used herein refers to a malignant neoplasm of the cervix uteri or cervical area. A typical treatment consists of surgery (including local excision) in early stages and chemotherapy and radiotherapy in advanced stages of the disease. Following chemotherapy and radiotherapy, the cervical cancer may relapse as a subtype of cervical cancer resistant to at least one of the presently available chemotherapies or radiotherapies.

Abnormal cells can be identified and differentiated from normal cells by evaluating one or more biomarkers within the cells.

The term “biomarker” refers to a macromolecule that is present in a cell being analyzed, and includes nucleic acids (e.g., DNA, mRNA, microRNA or other non-coding RNA), proteins (e.g., enzyme, receptor, or antibody), carbohydrates, lipids, macrocycles, and/or combinations thereof. A biomarker can include macromolecules that are normally present in the sample of cells being evaluated or can be macromolecules that are derived from foreign or infectious origins, such as a virus or bacteria. Biomarkers can be correlated with a disease state or pathogen. In certain embodiments, a specific biomarker may be deliberately evaluated by an observer or instrument to reveal, detect, or measure the presence or frequency and/or amount of a specific condition, event or substance. For example, molecular markers are specific molecules, such as proteins or protein fragments, whose presence within a cell or tissue indicates a particular disease state.

As used herein, the term “genetic material” is intended to mean materials comprising or formed predominately of nucleic acids. The term specifically is intended to encompass, deoxyribonucleic acids (DNA) or fragments thereof and ribonucleic acids (RNA) or fragments thereof. The term also can be used in reference to genes, chromosomes, and/or oligonucleotides and can encompass any portion of the nuclear genome and/or the mitochondrial genome of the human body.

In certain variations, the biomarker can be a polynucleotide sequence of DNA or RNA or a polypeptide sequence. A DNA biomarker can be an entire chromosome, a chromosome region, or a fragment or complement of such sequences. Similarly, an RNA biomarker can contain the entire or partial sequence of any of the nucleic acid sequences of interest. A protein biomarker can be directed to the entire or partial amino acid sequence of the protein. In a specific embodiment, the biomarker is a nucleic acid sequence representing a segment of a human chromosome.

### Chromosomal Regions

As used herein, the term “chromosome region” refers to a portion of a chromosome. The term also can be used in relation to specific oligonucleotides that have sequences that correspond to a portion of the human genome. The location of the nucleic acid polymer within the genome can be defined with respect to either the chromosomal band in the human genome or one or more specific nucleotide positions in the human genome. Several chromosome regions have been defined by convenience in order to refer to the location of genes, for example the distinction between chromosome region p and chromosome region q. In diploid organisms, homologous chromosomes get attached to each other by the centromere. The centromere divides each chromosome into two regions: the smaller one, which is the p region, and the bigger one, the q region. At both ends of a chromosome is a telomere, and the areas of the p and q regions close to the telomeres are the subtelomeres, or subtelomeric regions. The areas closer to the centromere are the pericentronomic regions. Finally, the interstitial regions are the parts of the p and q regions that are close to neither the centromere nor the telomeres, but are roughly in the middle of p or q. The chromosomal region may be further defined by reference to the conventional banding pattern of the chromosome. For example, 3p11.2 refers to chromosome 3, p arm, with the numbers that follow the letter representing the position on the arm: band 1, section 1, sub-band 2. The bands are visible under a microscope when the chromosome is suitably stained. Each of the bands is numbered, beginning with 1 for the band nearest the centromere. Sub-bands and sub-sub-bands are visible at higher resolution. As a further example, 3p11.2-p14.1, refers to the region on the p arm of chromosome 3 from band 1, section 1, sub-band 2 to band 1, section 4, sub-band 1.

The term “CEN” or “Cen” refers to a Centromere and the term “CEP” refers to a Centromere Enumerating Probe. Certain embodiments of the present disclosure are directed to the use or detection of a CEP7 probe. Thus, as used herein, CEP7 refers to a probe that recognizes and hybridizes to chromosome the centromere of chromosome 7 (CEN7).

In some embodiments, abnormal cells can be detected and differentiated from normal cells by evaluating the dosage of chromosomal regions within a cell sample. Dosage generally refers to the number of copies of a chromosomal region, or portion thereof, or a gene present in a cell or nucleus. Thus, a chromosomal region dosage represents the number of copies of a particular chromosomal region, or portion thereof, in a cell or nucleus. Likewise, a gene dosage refers to the number of copies of a particular gene in a cell or nucleus. The term dosage encompasses equivalents, gains, and losses.

As used herein, “gain” of a chromosomal segment (e.g., “gain of 3q” or “3q gain”) refers to multiplication (amplification) of all or any part thereof of the chromosome segment resulting in increased copy number of the segment. In one embodiment, “gain of 3q” is multiplication (amplification) within 3q26.

As used herein, “loss” of a chromosomal segment (e.g., “loss of 3q” or “3q loss”) refers to a deletion of all or any part thereof of the chromosome segment resulting in decreased copy number of the segment.

As used herein, “tetraploidy” or “tetra-ploidy” refers to a duplication of the chromosomal complement, or four (4) times the haploid number of chromosomes in the nucleus. Tetraploidy can be seen during the normal process of cell division. Tetraploidy may also be caused by a response to reactive conditions (such as benign infections, inflammation, etc.) or may be associated with cervical dysplasia.

In certain embodiments, chromosomal regions that are analyzed for gains and losses include those regions involved in cervical cancer, including those identified and discussed in patent documents: US2011/0224088 by Lyng et al.; US2012/0295807 by Rosenberg et al.; US2014/0079836 by McDaniel; WO2006/081621 by Hammer; WO2012/033828 by Chaganti et al.; WO2014/072832 by Lyng et al.; US2014/0045915 by Skog et al.; U.S. Pat. No. 8,603,746 by Endress et al.; U.S. Pat. No. 8,603,747 by Endress et al.; and in publications including: Rajkumar et al.: “Identification and validation of genes involved in cervical tumourigenesis.” BMC Cancer (2011) 11:80; Rajkumar et al.: Identification and validation of genes involved in cervical tumourigenesis. BMC Cancer (2011) 11:80; and in various public databases including: http://www.ncbi.nlm.nih.gov; http://www.expasy.org; http://www.genscript.dk; http://atlasgeneticsoncology.org (all of which are hereby incorporated by reference in their entireties). In specific embodiments, the chromosomal regions include the regions of human chromosome 3, 5, 7, and 20. In a particular embodiment, chromosomal regions include the regions and genes identified in Table 6 (Chromosome 3); Table 7 (Chromosome 5); and Table 8 (Chromosome 20) and Cen7 on chromosome 7. In a more specific embodiment, chromosomal regions include 3q26, 5p15, Cen7, and/or 20q13.

### Probes

As used herein, the term “probe” is intended to mean any molecular structure or substructure that hybridizes or otherwise binds to a genomic region. Probes can be labeled with any substance that can be attached to the probe so that when the probe binds to a corresponding site a signal is emitted or the labeled probe can be detected by a human observer or an analytical instrument. Labels envisioned by the disclosed method can include any labels that emit a signal and allow for identification of a component in a sample. Non-limiting examples of labels encompassed by the disclosed method include fluorescent moieties, radioactive moieties, chromogenic moieties, and enzymatic moieties.

The disclosure also provides methods of utilizing the probes for identifying biomarkers indicative of HPV-associated cancer. Various materials can be used in carrying out the methods disclosed herein and the following discussion provides only certain embodiments encompassed by the invention. Further embodiments also are intended to be encompassed by the invention.

In certain embodiments, the disclosed method can provide a probe set or panel of probes for detecting biomarkers in a sample indicative of HPV-associated precancer or cancer. Particularly, the probe set comprises a plurality of labeled, distinct genomic regions, wherein each of the distinct genomic regions can be individually capable of hybridizing to material present in a sample. Specifically, the genomic regions in the probe set can be regions wherein an alteration therein is correlated to one or more types of HPV-associated cancer. The probe set can be used in a FISH-based testing algorithm to identify biomarkers indicative of HPV-associated cancer and thus provide a tool for diagnosis and prognosis of HPV-associated cancers in various stages of the cancer cycle (e.g., precancer, early stage cancer, and late stage cancer).

As noted herein, the disclosed method can related to specific probes useful in identifying biomarkers indicative of HPV-associated cancer. Such probes can be prepared according to various methods not limited to the exemplary embodiments described herein. In certain embodiments, one or more probe sets commercially available can be used. In other embodiments, the inventive methods can be carried out using specially prepared probe sets. In still further embodiments, combinations of probe sets can be used. As used herein, the term “probe set” is intended to mean a single set and/or two or more sets, wherein each set can comprise a plurality of nucleic acids of varying lengths that are homologous or complementary to genomic regions (e.g., DNA fragments).

The probes of the disclosed method hybridize to genomic DNA, particularly a target genomic region as disclosed herein. It is recognized that for two single-stranded DNAs to hybridize to each, such as for example, a probe and a target genomic region as disclosed herein, one single stranded DNA must be complementary to the other DNA single stranded DNA. Thus, the probes of the disclosed method encompass nucleic acids that are complementary to either strand of the double-stranded DNA of the target genomic regions as disclosed herein. While the probes of the disclosed method can be fully complementary to all or at least a portion of a target genomic region of the disclosed method, the disclosed method encompasses probes that are not fully complementary to a target genomic region but that can specifically hybridize to the target genomic region under hybridization conditions disclosed herein or otherwise known one of skill in the art.

Probes directed to any chromosomal region can be utilized by the methods disclosed herein. Chromosomal probes include, nucleic acid probes that recognize chromosomal regions in 1q; 2q; 3q; 5p; 6p; 6q; 7; 8q; 9p; 9q; 10q; 11q; 12q; 16q; 17p; 18p; 19q; 20q and/or combinations thereof. Probes to: 1q; 12q; 19q; 11q; 6q; 17p; 7; 8q (detected in late stage dysplasia); 9q; 16q; 2q; 9p; 10q; 18p and any combination of probes thereof. According to specific embodiments of the aforementioned probe panel, probes to the 3q26 locus and 5p15 locus, including the Cri du Chat region, in addition to, probes to the following chromosomal loci can be used: 1q21-31; 20q12; 12q13-24; 19q13; 11q21; 7q11-22; 8q24 (detected in late stage dysplasia); 9q33-34; 16q23; 2q32; 9p22; 10q21-24; 18p11 and any combination thereof.

In one aspect, methods are disclosed for assessing a patient condition of cervical cell disorder which may include cervical dysplasia or cancer comprising: detecting, in a sample from a patient: a genomic amplification in chromosome 3q; a genomic amplification in chromosome 5p; a genomic amplification in chromosome 20q; a genomic amplification in chromosome the centromere of chromosome 7 (CEN7); and/or any combination thereof.

In a specific embodiment, the probes are FISH probes the FISH-based HPV-Associated Cancer Test (FHACT®) combination probe (manufactured by CGI Italia) was used, which contained the following probes: 3q26 (TERC) (red), 5p15 (D5S2095) (green), 20q13 (D20S911) (gold) and CEP7 (aqua) as described in WO 2012/033828, which is incorporated by reference in its entirety. The FHACT® combination probe set is a four color FISH Probe that can be used for cervical cancer screening as additional triage before referral for colposcopy. FHACT® can be used on leftover thin prep specimen (no resampling) and conventional Pap smears.

### Abnormalities

The present disclosure is directed to a method for screening and detecting abnormal cells in a sample by evaluating the presence, absence, or amount of a biomarker in the cells of the sample. In embodiment, abnormal cells can be detected and differentiated from normal cells by identifying the presence of a particular biomarker. For example, an abnormal cell that has been infected by a virus can be differentiated from a normal cell that has not been infected, by detecting the presence of viral proteins or nucleic acids within the abnormal cell. In another embodiment, abnormal cells can be detected and differentiated from normal cells by identifying the absence of a particular biomarker. In yet another embodiment, an abnormal cell can be detected and differentiated from normal cells by comparing the relative amounts of a particular biomarker within the cells. In variations of this embodiment, abnormal cells can include cells that have an increase or decrease in the biomarker compared to normal cells.

Genetic aberrations can be observed in a sample of cells cytologically, by measuring genetic abnormalities either as increase or decrease in gene regions. The methods discussed herein can directly identify abnormalities in the DNA of cervical cells by detecting aberrant regions in the chromosome. When greater than, or less than, the expected number of chromosomal regions are observed, a cell sample can be diagnosed as diseased and DNA damage can be diagnosed before dysplasia can be observed cytologically. Subjects with these abnormalities can have a poor prognosis and can be at high risk to develop more advanced cervical disease.

The disclosed system and method are useful for detecting abnormalities in cervical cells from a human patient including, but not limited to, cervical cancer as well as a variety of viral, parasitical or bacterial infections associated with sexually transmitted infections, such as candidiasis, chancroid, chlamydia, cytomegalovirus, granuloma inguinale, gonorrhea, hepatitis, herpes, human immunodeficiency virus (HIV), human papillomavirus (HPV), syphilis and/or trichomoniasis.

The present disclosure is generally applicable to any one or more types of HPV-associated precancers and cancers including, but not limited to, precancers and cancers of the cervix, anus, vulva, vagina, penis, oropharynx, and pharynx. The methods disclosed herein can be performed subsequent to or in lieu of ASCUS/HPV+ or LSIL Pap tests/results, among other abnormal results from cytology testing, in order to provide more specific information about a patient's risk of disease progression. One aspect of the disclosed method is directed to the identification of gain in copy number of chromosomal regions associated with cancer, and in particular cervical cancer. The disclosed method is useful for screening and/or detecting the presence of cervical cell disease, including cervical cancer or cervical dysplasia, in a patient. Overall then, there exist genomic abnormalities (gain of 3q, 5p, and/or 20q) that are shared to some extent in several HPV-associated diseases, and for which there is some preliminary evidence suggesting an early role in carcinogenesis.

As used herein, “cytogenetic abnormality” when used in singular or plural, shall mean an alteration in the human genome that can be detected by examination of the chromosomes. A “cytogenetic abnormality” is also referred to herein as a “chromosomal abnormality”.

As used herein, “cytogenetic assay” shall mean a laboratory assay that examines chromosomes.

### Detection

Detection of abnormal cells can be performed using a variety of techniques depending on the biomarker being analyzed. Methods for detecting nucleic acids include, polymerase chain reaction (PCR); real-time PCR; Northern blotting; Southern blotting; in situ hybridization (ISH); chromogenic in situ hybridization (CISH), fluorescence in situ hybridization (FISH) including DNA-FISH, RNA-FISH, combined DNA and RNA-FISH; RNA in situ hybridization (RNAscope®); methylation-specific fluorescence in situ hybridization (MeFISH); microarrays; comparative genomic hybridization (CGH); and next-generation sequencing.

In a specific embodiment, the detectable marker of the probe can emit a fluorescent signal or the probe may be chromogenic. The probes are hybridized using fluorescent in situ hybridization (FISH). FISH is a cytogenetic technique used to detect or localize the presence or absence of specific DNA sequences on chromosomes. FISH uses fluorescent probes that bind to parts of the chromosome with which they show a high degree of sequence similarity. Fluorescence microscopy can be used to find out where the fluorescent probe binds to the chromosome. In situ hybridization is a technique that allows the visualization of specific nucleic acid sequences within a cellular preparation. Specifically, FISH involves the precise annealing of a single stranded fluorescently labeled DNA probe to complementary target sequences. The hybridization of the probe with the cellular DNA site is visible by direct detection using fluorescence microscopy.

In instances where additional genetic material is required for testing, the genome may be amplified or detected by Polymerase Chain Reaction (PCR).

FISH can also be performed on liquid cytology specimens such as SUREPATH® or THINPREP® specimens for hybridization of DNA probes. SUREPATH® is available from Becton-Dickinson of Sparks, Md. THINPREP® is available from Hologic Laboratories of Bedford, Mass.

The present disclosure is, in certain embodiments, directed to a fluorescence in situ hybridization (FISH)-based HPV-associated cancer detection test (FHACT®) to detect genomic abnormalities in cervical, anal, vulval, vaginal, penile, oropharyngeal, and pharyngeal specimens. Further embodiments provide for use of the test in HPV-associated cancer screening programs.

In specific embodiments, the disclosed method provides a robust, sensitive, and specific FISH-based test that, together with standard cytology and HPV-typing, can provide for accurate detection of precancer and cancer in cytology specimens. Such test can significantly impact standard-of-care recommendations in HPV-associated cancer screening programs and can identify patients requiring additional follow-up and treatment.

The present disclosure provides for the assessment of genomic alterations in the diagnosis and prognosis of precancer, particularly HPV-associated cancer. In particular, the disclosure provides the ability to use hybridization technology, such as fluorescence in situ hybridization (FISH), as a clinical tool for the diagnosis and prognosis of HPV-associated cancer.

In one aspect, a probe set for detecting biomarkers in a sample that are indicative of HPV-associated cancer are used. In certain embodiments, the probe set can comprise a plurality of labeled, distinct genomic regions, such as DNA fragments (including bacterial artificial chromosomes (BACs)). Preferably, each of the distinct genomic regions is individually capable of hybridizing to material present in the sample. Moreover, the genomic regions in the probe set can be regions wherein an alteration therein is correlated to one or more types of HPV-associated cancer (i.e., are biomarkers indicative of HPV-associated cancer progression).

In another aspect, biomarkers in a sample indicative of HPV-associated cancer progression are detected. Such methods can be useful to identify precancer cells, formations, or the like, as well as early and/or late stage cancer. Certain embodiments include the following steps: (a) providing a probe set as described herein; (b) providing the sample with genetic material therein; (c) hybridizing the genetic material in the sample with the probe set; (d) analyzing the hybridization pattern of the genetic material in the sample to the probe set to detect patterns indicating the presence of alterations in the genetic material from the sample; and (e) identifying any detected alterations as biomarkers indicative of HPV-associated cancer progression. Fluorescence in situ hybridization (FISH) is utilized in certain embodiments.

In interphase FISH, a single-stranded fluorescent-labeled nucleic acid sequence (probe) complementary to a target genomic sequence is hybridized to metaphase chromosomes and interphase nuclei to detect the presence or absence of a given abnormality (Patel A S, Hawkins A L, Griffin C A, Curr. Opin. Oncol. (2000), 12(1):62-7; and Carpenter N J, Semin. Pediatr. Neurol. (2001), 8(3): 135-46). FISH can be applied to non-dividing (interphase) cells and a variety of specimen types. Depending on the color scheme and placement of the probes (spanning or flanking the genomic region of interest), interpretation of hybridized nuclei preparations can involve counting of hybridization signals per nucleus (genomic gain/loss), identification of fusion hybridization signals (rearrangement), or identification of signals that break apart (rearrangement). For the most part, in a clinical laboratory setting, FISH is considered an adjunct to traditional G-banding metaphase chromosome analysis. Even in this capacity, the impact of FISH-based assays on patient management is well established for a broad range of cancers for both diagnostic and prognostic purposes. Two FISH-based tests that have been FDA-approved in cancer are: PATHVYSION® (Abbott Molecular, Inc./Vysis, Inc.) for the detection of HER2 amplification in breast cancer to assist in treatment decisions, and UROVYSION™ (Abbott/Vysis) for the detection of aneuploidy associated with bladder cancer in urine specimens. In these tests, a FISH-based assay is being utilized in clinical management of patients in conjunction with morphologic examination (pathology and cytology respectively) and not metaphase chromosome analysis. In addition, both assays involve enumeration of signals per nucleus (cut-offs established by the manufacturer based on large cohort studies), which lends itself for automation using systems such as the Metafer (MetaSystems). Such systems are currently in routine use in clinical laboratories for assays such as UROVYSION™. Thus, commercial precedence exists for the use of highly sensitive FISH-based assays in diagnostic and prognostic clinical settings in solid tumors.

While HPV infection plays a major role in the development of cervical, vaginal, and anal cancer, additional host oncogenic events are involved. Molecular cytogenetic and genetic studies have identified a number of genomic abnormalities that are shared between these cancer types that potentially harbor oncogenes or tumor suppressor genes. For several of these regions, candidate genes have been suggested though none have experimentally been confirmed to have such a role. Despite this, these abnormalities serve as biomarkers of HPV-associated cancers, but it is unknown at which stage in the etiology of these cancers, these abnormalities are observed. HPV-associated cancers are thought to follow a course from initial infection, to persistence of the infection, to progression into a precancerous lesion that ultimately becomes invasive cancer. For cervical cancer, there is reasonable evidence to suggest that gain of 3q is a genomic alteration that is associated with progression of the disease into a precancerous lesion and that detection of this abnormality in cervical cytology specimens may differentiate between lesions that will progress versus regress. There is also some preliminary evidence supporting a similar role for gain of 5p and 20q in cervical cancer progression.

The present disclosure can provide improved screening programs for HPV-associated cancers, particularly through the identification of biomarkers associated with HPV-associated cancer progression. In specific embodiments, as described herein, the disclosed method provides for the use of FISH-based assays in the evaluation of biomarker indicative of HPV-associated cancer in cervical and anal cytology specimens, such as the gain of 3q, 5p, 20q, centromere 7, and combinations thereof. The disclosure also can provide for determining whether detected genetic alterations are biomarkers of HPV-associated cancers that can successfully stratify patients into those that require additional treatment versus those who do not. In particular embodiments, this can be accomplished through use of a robust, sensitive, and specific FISH-based HPV-associated cancer detection test (FHACT®) that can significantly contribute to clinical decision making in patients with abnormal cytology diagnoses, impacting clinical management and cost of care. The disclosed method also can allow for evaluating the commonality of genetic alterations in HPV-associated cancers and obtaining valuable information on possible common roles of these abnormalities in the etiology of the diseases.

### Cell Sample Collection and Preparation

Various methods can be used in specimen collection and preparation.

Cells recovered and isolated from specimens or samples collected from patients can be fixed on slides. Specimens can be retrieved using various techniques known in the art. In one embodiment specimens can be retrieved from THINPREP® and/or SUREPATH® samples. SUREPATH® is a Pap test used for the screening of cervical cancer. SUREPATH® has various collection devices to collect Pap samples from a patient. Some have detachable heads that hold the sample, are directly detached and put into a vial that is sent for screening, enabling 100% of sample to be available for processing. A liquid-based Pap test using thin-layer cell preparation process called the BD SUREPATH® liquid-based Pap test which claims an increase in detection rate compared to the conventional Pap smear is used with the SUREPATH® collection devices such as the broom-like device or the brush/spatula with detachable heads, as disclosed in U.S. patent application Ser. No. 11/521,144, incorporated herein by reference in its entirety. The THINPREP® Pap is a liquid-based cytology method. A sample of the cervical cells is rinsed into a vial instead of a smear onto a slide thus preventing clumping of cells. The cells are separated in a laboratory to eliminate blood and mucus and the cells to be studied are then placed on a slide for studies to detect cancerous cells.

The samples may also comprise analysis of tissue from cervical biopsies, punch biopsies, “soft” biopsies (Histologics™ LLC) surgical procedures including LEEP, hysterectomy, CONE biopsy, ECC. The sample may be prepared from tissue or cells removed from the cervix, vagina or vulva.

Cervical cytology specimens for FHACT® can be received in PreservCyt™ and SurePath™, alcohol-based preservation media used routinely for the preservation of cervical specimens in preparation for cervical thin-layer cytology. For FISH, the specimen cells preferably can be transferred into Carnoy's fixative, (3:1 methanol:acetic acid), which removes most of the cytoplasm leaving nuclei open to hybridization with the DNA probe. The Carnoy's fixative evaporates rapidly facilitating the spreading of nuclei when making air-dried slides. Thus the cells of the coded specimen (approximately 0.5 to 1 ml) can be pelleted (such as by centrifugation), re-suspended in fixative, and left for about 30 minutes. Alternately, the cells can be stored overnight of longer (e.g., at 4° C.). The fixative then can be changed at least two times just prior to use or for longer storage (e.g., −20° C. for up to 3 years). In specific embodiments, about 0.5-1.0 ml residual cytology specimen can be sufficient material (nuclei) for an average of about 4-20 hybridization areas having a dimension of about 18 mm².

### Hybridization

Chromosomal regions disclosed here are identified using in situ hybridization. Generally, in situ hybridization comprises the following major steps: (1) fixation of tissue or biological structure to be analyzed; (2) pre-hybridization treatment of the biological structure to increase accessibility of target DNA, and to reduce nonspecific binding; (3) hybridization of the mixture of nucleic acids to the nucleic acid of the biological sample or tissue; (4) post-hybridization washes to remove nucleic acid fragments not bound in the hybridization and (5) detection of the hybridized nucleic acids. Hybridization protocols for the applications described herein are described in U.S. Pat. No. 6,277,563, which is incorporated by reference in its entirety.

From samples, the target DNA can be denatured to its single stranded form and subsequently allowed to hybridize with the probes. Following hybridization, the unbound probe is removed by a series of washes, and the nuclei are counterstained with DAPI (4,6 diamidino-2-phenylindole), a DNA-specific stain. Hybridization of the DNA probes can be viewed using a fluorescence microscope equipped with appropriate excitation and emission filters allowing visualization of the aqua and gold fluorescent signals. Enumeration of CEN 7, and chromosomal signals is conducted by microscopic examination of the nuclei.

The clinical test disclosed herein can use several biomarkers in combination for the early detection of cervical cancer and is important because current morphology based screening and detection methods have significant limitations. Identification of chromosomal regions, including 3q, 5p, and/or 20q, amplification and other cytogenetic abnormalities can more precisely and accurately identify patients at risk for developing cervical cancer and help them receive earlier treatment.

Prior to hybridization, slides can treated be with pepsin (e.g., 0.004% in 0.01N HCl) at for a time of about 15 minutes at a temperature of about 37° C., washed twice in PBS at room temperature (T) for 5 minutes each, post-fixed in 1% formaldehyde for about 5 minutes at RT, dehydrated in an ethanol series (e.g., 70% and 100%) for 2 minutes each at RT, and air-dried. The FHACT probe cocktail in hybridization mix (5 μl) then can be applied to each target area of the slide (a circle), coverslipped, and sealed (such as with rubber cement). The probe/hybridization mix and specimen can be co-denatured (e.g., at about 80° C. for 2 minutes) and incubated overnight in a humidified chamber (e.g., at about 37° C.). After removal of the rubber cement and the coverslip, the slide can be submitted to two washes in 2×SSC plus 0.1% Tween-20 (e.g., 45° C. for about 5 minutes), and rinsed briefly in distilled water at RT. The slides then can be air-dried, DAPI counterstain applied, and coverslipped. Slides preferably are kept in a light-sensitive box until scoring is performed.

For each hybridization batch, the control slide initially can be scored using any suitable equipment type, such as an epi-fluorescence microscope equipped with filters to view the red, green, blue, and gold hybridization signals arising from the labels used in this embodiment. The microscope also can include a CCD camera. An exemplary operating system is the Isis Imaging Software (available from Metasystems).

The slide first can be examined for cell density, background, nuclear morphology, and hybridization signal strength. Using established criteria (e.g., derived from experience in performing FISH with other probes on clinical specimens), the quality of hybridization can be ranked and, if suitable for analysis, is scored. In one method for scoring, 300 or more nuclei are consecutively scored where nuclei are not scored if they are: 1) overlapping such that the signals belonging to each nucleus cannot be distinguished; 2) are scratched or otherwise physically damaged; 3) are partially covered by fluorescent debris which might obscure signals; 4) have signals which are pale or irregular and cannot be distinguished from background; and 5) do not have at least one red, one green, one blue, and one gold signal (i.e., at least one signal for each label color used). For scoring, each signal must be on or touching the DAPI-stained nucleus, be larger than background spots, and be a single spot, a closely-spaced doublet (less than one signal-width between), a closely-spaced cluster, or a continuous string. The nuclei are scored according to the signal patterns obtained for each probe set, where the expected normal pattern would be two signals of each color. Once it is determined that the controls are within the established ranges, the specimen slides are scored in a manner that is essentially the same as the control slide except that 300 or more nuclei are scored. In this embodiment, the patterns of hybridization (# red signals; # green signals; # gold signals; # blue signals) and the number of cells exhibiting these patterns are recorded. The number of cells with an abnormal pattern (e.g., more than two signals of red, green, gold, and/or blue) with the respective abnormality are calculated.

The slides can be pre-treated manually (optionally, pre-treated using VP2000 (Abbott Molecular, Inc., Des Plaines, Ill.)), hybridized manually (optionally, hybridized using Thermobrite Denaturation/Hybridization System (Abbott Molecular, Inc.)), and washed manually. Using microscopy, abnormal cells can be selected, and probes can be enumerated. Preferably, an automated procedure is used. An automated procedure can involve collecting and fixing cells in PreservCyt (Hologic, Inc., Bedford, Mass.). ThinPrep slides (Hologic, Inc.) can be prepared, pre-treated using VP2000 (Abbott Molecular, Inc.), and hybridized using Thermobrite Denaturation/Hybridization System (Abbott Molecular, Inc.). The slides can be washed and, using microscopy, abnormal cells can be identified, and probes can be enumerated. Cells can be pre-scanned, sorted and imaged, which allows for automatic probe enumeration and remote review. The use of ThinPrep results in cleaner background, reduced cell loss, larger and flatter cell morphology, and better signal quality.

### Analysis

In situ hybridization is a technique that allows the visualization of specific nucleic acid sequences within a cellular preparation. Traditionally the visualization of probe signals has been performed manually by highly-trained personnel. However, it is possible to adapt current technology to automate the image acquisition and analysis process. Microscopes on the market today, such as those manufactured by Carl Zeiss, Leica, Nikon, and Olympus, allow the user to capture digital images of the field of view within the specimen/slide on the microscopy stage. Some of these manufacturers have software available for the automated acquisition of images from specimens/slide. In addition, several entities (Ikonisys, Metasystems, Bioimagene, BioView, Aperio, Ventana, among others) have created software platforms specifically for use in commercial laboratories. Some of these entities have systems that include both a microscopy platform and the automated imaging software, including the Ikoniscope Digital Microscopy System by Ikonisys and Metafer and Metacyte by Metasystems.

The type and source of the specimen to be analyzed directly impacts the analysis process and methodology. Each tissue type has its own biology and structure plus each cancer develops differently with different factors affecting the rate of carcinogenesis. Therefore, the present disclosure provides for several methods for automated image acquisition and analysis of specimens.

It is an embodiment of the system and method to be used in conjunction with specimens in liquid suspension that can be placed onto a microscope slide in an even, monolayer of cells, this includes liquid-base cytology specimens such as THINPREP® and SUREPATH® plus any fine-needle aspirate (FNA), sputum, or swab-based collection. This automated method screens the entire area covered by cells on the FISH prepared slide and utilizes the DAPI-stain to identify cellular nuclei. The system then enumerates each probe signal within the DAPI-stained region and records the copy number of each probe identified. The software system continues its automated scoring of cells and chromosomal copy number within each cell until it obtains results of at least 1000 cells. Once the 1000 cell threshold is reached, the software can categorize each cell imaged and counted into a category based upon the copy number of each chromosome identified. A normal cell with two copies of each probe (e.g., 3q, 5p, 20q, and CEN7) would be placed into a 2,2,2,2 category. Abnormal cells would be identified by their probe signal patterns. For instance, a cell with two copies of the CEN7 probe, 5 copies of the 3q probe, 3 copies of the 5p probe, and 4 copies of the 20q probe can be placed in the 2,5,3,4 category. Once all of the imaged cells are categorized, the specimen can be evaluated relative to the positive/negative disease threshold. All cells identified as abnormal by the automated imaging system can be reviewed and verified manually by trained personnel before test results are communicated to a physician. The present disclosure further provides for automated verification. Specific cell threshold numbers can vary by specimen type and collection method. In addition, the software can be adapted to reflect biological (cell shape, cell size, DNA content of the nucleus, proximity of cells to each other, cell type, etc.) or disease related differences (number of loci with abnormal number, the number of abnormalities at a locus within a single cell, relationship of an abnormality to survival or treatment response). This method and system can be used on a representative sampling of area covered by cells on the slide instead of the entire area, typically this is performed by imaging multiple fields of view or a path based on cellular density until the minimum imaged cell threshold is met.

Cells identified as abnormal by the automated imaging system can be reviewed and verified manually by trained personnel before test results are communicated electronically via methods known in the art to a physician. Specific cell threshold numbers can vary by specimen type and collection method. In addition, the software can be adapted to reflect biological (cell shape, cell size, DNA content of the nucleus, proximity of cells to each other, cell type, etc.) or disease related differences (number of loci with abnormal number, the number of abnormalities at a locus within a single cell, relationship of an abnormality to survival or treatment response). The present embodiments can be used on a representative sampling of area covered by cells on the slide instead of the entire area, typically this is performed by imaging multiple fields of view or a path based on cellular density until the minimum imaged cell threshold is met. Only a subset of the rank-ordered abnormal cells can be reviewed relative to the positive/negative test threshold as long as the clinical and disease significance is known for the subset. Typically the subset is the most abnormal 25 or 50 cells within the specimens, but other subsets can be identified and utilized depending on the specimen source, collection method, and disease.

The scoring data can be analyzed by calculating the number of any one of the signals (e.g. 3q, 5p, 20q, or CEN7) and dividing by the total number of nuclei scored; recording that number in the chart at the top of the Score Sheet. A result greater than 2 recorded and reported as amplified for any given probe.

The scoring data is analyzed by adding the number of any one of the signals (e.g., 3q, 5p, 20q, or CEN 7) and dividing by the total number of nuclei scored. A result greater than 2 can be reported as amplified for the given probe. Images are named by the specimen number and slide number and saved.

### Automated Systems

Automated systems include systems for sample preparation, slide preparation, probe denaturation/hybridizing, microscopy platforms, and automated imaging software.

Typical microscopic automation can provide for efficient and expedient biological sample analysis. Automatic microscopy can include, but is not limited to, robotic microscopic systems, automatic operation, automated slide scanning, automated stage, automated slide cassettes and handling systems, and computer software systems to facilitate detection and analysis of fluorescent signals.

It is yet another embodiment to provide for an automated microscope and system to perform each of the steps of the method disclosed herein. It is an embodiment whereby each of the steps is carried out without manual intervention. It is also an embodiment of the invention, for the microscope to read a patient identified, e.g. barcode, on the slide for entry into a database prior to scanning so that the results of the method can be indexed according to each patient identifier.

It is an embodiment to provide for automated image analysis of the signal from the FISH probe. Microscopes can allow for automated capture of digital images of the field of view within the specimen/slide on the microscopy stage. Such manufacturers include Carl Zeiss, Leica, Nikon and Olympus. Also, the method provides for software platforms for automated image analysis such as microscope-software systems developed by such entities Applied Spectral Imaging of California, as Ikonisys of Connecticut, Metasystems of Massachusetts and Germany, Bioimagene of California, and Bioview of Massachusetts and Israel, among others. Such automated systems may apply to viewing 3q chromosomes alone or in combination with 5p abnormalities in the patient sample.

The type and source of the specimen to be analyzed directly impacts the analysis process and methodology. Each tissue type has its own biology and structure plus each cancer develops differently with different factors affecting the rate of carcinogenesis. In order to account for variation in cell biology, morphology and structure, the method can distinguish between epithelial and other cells and structures to avoid unwanted artifacts in the image. The software system of the invention can account for these different factors. Morphology can be automatically imaged where cells morphogenically suspicious for malignancy can be further analyzed for morphological abnormalities including, but not limited to, pyknosis, large nuclear size, irregular nuclear shape, and patchy DAPI staining Therefore, the system can begin with cells that appear morphologically abnormal before counting normal cells. If few morphologically abnormal cells are present, cells which are the largest or have the largest detectable nuclei are scanned and analyzed. Overlapping cells that cannot be distinguished are not counted.

In one embodiment, cells identified as abnormal by the automated system can be communicated electronically via methods known in the art to a physician or other user.

In yet another embodiment, the system and method captures an image used alternatively for scoring by (1) identifying the image sample number and recording the image used (2) visualizing the signal colors separately (3) analyzing and recording the signal patterns for individual nuclei, selecting the appropriate nuclei based on the criteria described in preceding paragraph and (4) recording the signal numbers.

### Specific Embodiments

(1) A method for identifying an abnormal sample of cells comprising:
a) hybridizing a set of chromosomal probes to the sample, wherein the set comprises probes to 3q26, 5p15, CEP7, and 20q13;
b) evaluating cells of the sample to detect and quantify the presence of each probe in the set;
c) categorizing the evaluated cells of the sample as normal or abnormal, wherein the normal cells contain exactly two copies of each probe in the set and the abnormal cells do not contain exactly two copies of each probe in the set;
d) calculating the percentage of the abnormal cells in the evaluated cells of the sample; and
e) identifying the sample of cells as abnormal if the percentage of abnormal cells in the evaluated cells is greater than or equal to a cut-off value of 0.3%.
   (2) The method of (1), wherein the sample of cells is a sample of cervical, vaginal, or anal cells.
   (3) The method of (2), wherein the abnormal cells are selected from the group consisting of: cells having a single gain, cells having multiple gains, tetra-ploid cells, and combinations thereof.
   (4) The method of (3), wherein a minimum of 1,000 cells in the sample are evaluated.
   (5) The method of (4), wherein the sample of cells is classified as abnormal if:
i. the percentage of cells having a single gain is ≧0.3%;
ii. the percentage of cells having multiple gains is ≧0.7%; or
iii. the percentage of tetra-ploid cells is ≧0.8%.
   (6) The method of (4), wherein the sample of cells is classified as abnormal if:
i. the percentage of cells having a single gain is ≧0.7%;
ii. the percentage of cells having multiple gains is ≧1.0%; or
iii. the percentage of tetra-ploid cells is ≧1.1%.
   (7) The method of (4), wherein the sample of cells is classified as abnormal if:
i. the percentage of cells having a single gain is ≧1.2%;
ii. the percentage of cells having multiple gains is ≧0.7%; or
iii. the percentage of tetra-ploid cells is ≧0.8%.
   (8) The method of (4), wherein the sample of cells is classified as abnormal if:
i. the percentage of cells having a gain in 3q26 is ≧1.3%;
ii. the percentage of cells having a gain in 5p15 is ≧1.2%;
iii. the percentage of cells having a gain in CEP7 is ≧1.0%;
iv. the percentage of cells having a gain in 20q13 is ≧1.0%;
v. the percentage of cells having multiple gains is ≧1.3%; or
vi. the percentage of tetra-ploid cells is ≧1.5%.
   (9) The method of (4), wherein the sample of cells is classified as abnormal if:
i. the percentage of cells having a gain in 3q26 is ≧2.2%;
ii. the percentage of cells having a gain in 5p15 is ≧3.2%;
iii. the percentage of cells having a gain in CEP7 is ≧1.6%;
iv. the percentage of cells having a gain in 20q13 is ≧0.9%.
v. the percentage of cells having multiple gains is ≧1.0%; or
vi. the percentage of tetra-ploid cells is ≧1.2%.
   (10) The method of (1), wherein the steps of the method are performed manually.
   (11) The method of (1), wherein the steps of the method are performed by an automated system.
   (12) The method of (11), further comprising the step of verifying steps (b)-(e) manually.
   (13) The method of (11), further comprising the step of verifying steps (b)-(e) manually anytime an abnormal cell having a multiple gains is detected by the automated system.
   (14) A method for detecting an abnormal sample of cervical cells comprising:
a) hybridizing a first nucleic acid probe to a target nucleic acid sequence on chromosome 3q of the cervical cells to form a first hybridization complex;
b) hybridizing a second nucleic acid probe to a target nucleic acid on chromosome 5p of the cervical cells to form a second hybridization complex;
c) hybridizing a third nucleic acid probe to a target nucleic acid on chromosome 20q of the cervical cells to form a third hybridization complex;
d) hybridizing a fourth nucleic acid probe to centromere of chromosome 7 (CEN7) to form a fourth hybridization complex;
e) evaluating cells within the sample to detect and quantify:
   i. the formation of the first hybridization complex on chromosome 3q;
   ii. the formation of the second hybridization complex on chromosome 5p;
   iii. the formation of the third hybridization complex on 20q;
   iv. the formation of the fourth hybridization complex on CEN7,
f) categorizing each cell within the evaluated cells as normal or abnormal, wherein
   i. the normal cell contains exactly two copies of 3q, 5p, 20q, and CEN7; and
   ii. the abnormal cell contains more than two copies of 3q, 5p, 20q, CEN7, or a combination thereof;
g) calculating the percentage of abnormal cells present in the evaluated cells of the sample; and
h) classifying the sample of cervical cells as abnormal if the percentage of abnormal cells in the evaluated cells is greater than or equal to a cut-off value of 0.3%.
   (15) The method of (14), wherein the abnormal cells are selected from the group consisting of: cells having a single gain, cells having multiple gains, tetra-ploid cells, and combinations thereof.
   (16) The method of (14), wherein a minimum of 1,000 cells in the sample are evaluated.
   (17) The method of (14), wherein the sample of cells is classified as abnormal if:
i. the percentage of cells having a gain in 3q26 is ≧1.3%;
ii. the percentage of cells having a gain in 5p15 is ≧1.2%;
iii. the percentage of cells having a gain in CEP7 is ≧1.0%;
iv. the percentage of cells having a gain in 20q13 is ≧1.0%;
v. the percentage of cells having multiple gains is ≧1.3%; or
vi. the percentage of tetra-ploid cells is ≧1.5%.
   (18) The method of (14), wherein the sample of cells is classified as abnormal if:
i. the percentage of cells having a gain in 3q26 is ≧2.2%;
ii. the percentage of cells having a gain in 5p15 is ≧3.2%;
iii. the percentage of cells having a gain in CEP7 is ≧1.6%;
iv. the percentage of cells having a gain in 20q13 is ≧0.9%.
v. the percentage of cells having multiple gains is ≧1.0%; or
vi. the percentage of tetra-ploid cells is ≧1.2%.
   (19) The method of (14), wherein the steps of the method are performed by an automated system.
   (20) A method for detecting an abnormal sample of cervical cells comprising:
a) hybridizing a first nucleic acid probe to a target nucleic acid sequence on 3q26 of the cervical cells to form a first hybridization complex;
b) hybridizing a second nucleic acid probe to a target nucleic acid on 5p15 of the cervical cells to form a second hybridization complex;
c) hybridizing a third nucleic acid probe to a target nucleic acid on 20q13 of the cervical cells to form a third hybridization complex;
d) hybridizing a fourth nucleic acid probe to centromere of chromosome 7 (CEN7) to form a fourth hybridization complex;
e) evaluating at least 1,000 cells within the sample to detect and quantify:
   i. the formation of the first hybridization complex on chromosome 3q26;
   ii. the formation of the second hybridization complex on chromosome 5p15;
   iii. the formation of the third hybridization complex on 20q13;
   iv. the formation of the fourth hybridization complex on CEN7,
f) categorizing each cell within the evaluated cells as normal or abnormal, wherein
   i. the normal cell contains exactly two copies of 3q26, 5p15, 20q13, and CEN7; and
   ii. the abnormal cell is selected from the group consisting of: a cell having a single gain, a cell having multiple gains, a tetra-ploid cell, and combinations thereof;
g) calculating the percentage of abnormal cells present in the evaluated cells of the sample; wherein the steps of (a)-(g) are performed manually or by an automated system, the method further comprising the step of
h) classifying the entire sample of cervical cells as abnormal if, the following percentages of abnormal cells are observed when the steps of (a)-(g) are performed manually:
   i. cells having a gain in 3q26 is ≧1.3%;
   ii. cells having a gain in 5p15 is ≧1.2%;
   iii. cells having a gain in CEP7 is ≧1.0%;
   iv. cells having a gain in 20q13 is ≧1.0%.
   v. cells having multiple gains is ≧1.3%; or
   vi. tetra-ploid cells is ≧1.5%;
or
i) classifying the entire sample of cervical cells as abnormal if, the following percentages of abnormal cells are observed when the steps of (a)-(g) are performed by an automated system:
   i. cells having a gain in 3q26 is ≧2.2%;
   ii. cells having a gain in 5p15 is ≧3.2%;
   iii. cells having a gain in CEP7 is ≧1.6%;
   iv. cells having a gain in 20q13 is ≧0.9%.
   v. cells having multiple gains is ≧1.0%; or
   vi. tetra-ploid cells is ≧1.2%.

In other embodiments, the present disclosure provides for kits for the detection of chromosomal abnormalities at the regions disclosed. In a preferred embodiment, the kits include one or more probes to the regions described herein and any combination of the disclosed probes. The kits can additionally include instruction materials describing how to use the kit contents in detecting the genetic alterations. The kits may also include one or more of the following: various labels or labeling agents to facilitate the detection of the probes, reagents for the hybridization including buffers, an interphase spread, bovine serum albumin and other blocking agents including blocking probes, sampling devices including fine needles, swabs, aspirators and the like, positive and negative hybridization controls and other controls as are known in the art.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

The following illustrative explanations of the figures and related examples are provided to facilitate understanding of certain terms used frequently herein, particularly in the examples. The explanations are provided as a convenience and are not limitative of the invention.

### EXAMPLES

### Example 1

### HPV 4C FISH Assay

a. Reagent Preparation

20×SSC: Powered 20×SSC (264 g) was mixed with 900 ml DI water using a magnetic stir plate and stir bar. The pH was adjusted to 7.0-7.5 with HCl. The total volume brought up to 1000 ml. The solution was filtered through a 0.45 μm pore filtration unit into the collection/storage bottle. This solution could be stored at room temperature for up to 6 months.

2×SSC: A volume of 20×SSC (100 ml) was mixed with 900 ml DI water. The solution was filtered through a 0.45 μm pore filtration unit into the collection/storage bottle. This solution could be stored at room temperature for up to 6 months. Any used solution was discarded at the end of the day.

2×SSC/0.1% NP-40: A volume of 20×SSC (100 ml) was mixed with 899 ml DI water and 1 ml of NP-40. The pH was adjusted to about 7.0 (+/−0.2). The solution was filtered through a 0.45 μm pore filtration unit into the collection/storage bottle. This solution could be stored at room temperature for up to 6 months. Any used solution was discarded at the end of the day.

70%, 85%, 100% Ethanol: Volumetric dilutions of 100% reagent alcohol were prepared with DI water and stored at room temperature. Reagent was used for a week and then discarded.

Protease Solution: Protease solution was prepared fresh for every FISH run using VP 2000 reagents (Abbott Molecular, Des Plaines, Iowa, USA). Protease powder (0.03 g) was added to 60 ml protease buffer in a small bottle. This solution was mixed and poured into plastic staining jar. This solution was discarded at the end of the run.

1% Formaldehyde Solution: A 10% solution of formalin (250 ml) was mixed with 1×PBS (740 ml), and 100×MgCl₂ (10 ml). The mixture was poured into a plastic staining jar. Any unused solution was stored at 2-8° C. for up to 6 months. Used solution was discarded after 1 week. The solution was discarded into a Formalin Waste bottle containing formalin neutralizer according to standard practice.

b. Probes

FISH probes were obtained from Cancer Genetics, Inc. (CGI). Specifically, for the FISH assay, the FHACT™ combination probe (manufactured by CGI Italia) was used, which contained the following probes: 3q26 (TERC) (red), 5p15 (D5S2095) (green), 20q13 (D20S911) (gold) and CEP7 (aqua) as described in WO 2012/033828.

c. Sample Preparation

Cell samples were obtained and prepared and slides were processed using the ThinPrep Pap Test and ThinPrep T2000 Processor according to the manufacturer's instructions (Hologic, Bedford, Mass., USA). Briefly, samples were prepared as follows:

Cell samples were obtained from a patient using the ThinPrep Pap Test. After collection, slides were prepared using a ThinPrep T2000 Processor using a yellow (UroCyt) filter and program #5 on the Processor. After being processed, the slides dropped into an empty vial and allowed to air dry before being analyzed by FISH.

Any sample remaining in the specimen vial was stored in the event that repeat FISH or additional testing was needed. Slides were be stored either in a refrigerator at 2-8° C. for up to several days, or in a freezer at −20° C. for long-term storage prior to being hybridized.

d. Slide Pretreatment

Specimen slides were pretreated as follows. First, an air-dried, room-temperature specimen slide was immersed into a solution containing 2×SSC at about 73° C. (+/−1° C.) for about 2 minutes (+/−0.5 minutes). Next, the slide was removed from the 2×SSC solution and placed into a protease solution (protease buffer containing fresh protease powder) at about 37° C. (+/−1° C.) for about 25 min (+/−1 min).

The slides were then air dried for about 5 min (+/−1 min) at room temperature. Slides were then fixed in 1% Formaldehyde solution for about 5 min (+/−1 min) at room temperature and then washed in 1×PBS for 5 min (+/−1 min) at room temperature. The slides were then dehydrated in 70% alcohol for about 1 minute, 85% alcohol for about 1 minute, and then 100% alcohol for about 1 minute. Slides were then allowed to air dry until completely dry.

e. Probe Denaturation/Hybridization

Hybridization was performed using Thermobrite Denaturation/Hybridization System according to the manufacturer's instructions (Abbott Molecular, Des Plaines, Iowa, USA),

FHACT DNA probe (CGI) and cDenHyb-2 were removed from a freezer and allowed to warm to RT. Each vial was vortexed to mix contents and spun briefly (about 1-3 sec) in microcentrifuge. Each vial was vortexed again to mix.

For each slide in the FISH run, 2 μl of probe was mixed with 4 μl of cDenHyb-2 in a microcentrifuge tube. The tube was vortexed to mix, spun briefly (about 1-3 sec), and vortexed again.

The probe mixture (5.5 μl) was applied to the cell spot on the slide and covered with a 15 mm round (siliconized) cover glass, carefully as to avoid creating air bubbles. The edges of the cover glass were sealed thoroughly with the rubber cement. The slides were then placed in Thermobrite (Abbott Molecular) and Humidity Strips were moistened with DI water.

The slide and the probe mixture were co-denatured for about 3 minutes at 78° C. and then hybridization took place for about 4 to about 18 hours at about 37° C. using program #3 (“FISH 4C”) on the Thermobrite.

f. Post-Hybridization Washing

A staining jar with 2×SSC/0.1% NP-40 was placed in a water bath and warmed to about 73° C. (+/−1° C.).

The slides were removed from the Thermobrite and the rubber cement was removed with forceps. The cover glass was then removed by soaking in 2×SSC at room temperature until the cover glass slid off.

The slide was placed in 2×SSC/0.1% NP-40 for about 1 hour 45 minutes at about 73° C. (+/−1° C.). After washing, the slides were air dried vertically out of direct light.

DAPI II (7-10 μl) was applied to the hybridized area and covered with 24×40 mm cover glass, avoiding air bubbles over the cell spot.

Hybridized slides were stored at about −20° C. for at least 20 minutes prior to viewing and protect from direct light.

### Example 2

### HPV 4C FISH—Manual Scoring

Slides were prepared from cervical or vaginal ThinPrep Pap Test specimen according to the preparation and hybridization protocol discussed in Example 1 and were stored at −20° C. until they were ready to be analyzed with the following procedure.

a. Slide Analysis

Probe signals and DAPI counterstain were visualized using the following fluorescent filters:
1. DAPI single bandpass (360 nm excitation, 460 nm emission): for viewing nuclei—in Filter Wheel position 1
2. Green single bandpass (496 nm excitation, 520 nm emission): for viewing 5p15 (D5S2095)—in Filter Wheel position 3
3. Red single bandpass (593 nm excitation, 612 nm emission): for viewing 3q26 (TERC)—in Filter Wheel position 2
4. Aqua single bandpass (431 nm excitation, 480 nm emission): for viewing chromosome 7 (Cen7)—in Filter Wheel position 4
5. Gold single bandpass (525 nm excitation, 551 nm emission): for viewing 20q13 (D20S911)—in Filter Wheel position 5
6. Triple cube—Red/Green/Aqua for scanning 3q26, 5p15, CEP7—in Filter Wheel position 6

The circular cell spot containing the cellular material was scanned using the above filters and oil objectives of 40× or 60×. Oil objectives of 60× and 100× were also used for enumerating signal counts.

The cell spot area was examined for cell density, background signal (noise), nuclear morphology, and hybridization signal strength to determine if slide is suitable for analysis. Slides were deemed insufficient for analysis based on the following criteria:
1. Slides with evaluable signals in less than 25% of the cells
2. Slides with less than 1000 evaluable epithelial cells
3. Slides having of many large clumps or abundance of bacteria

If the slide was insufficient for analysis due to the presence of many large clumps or abundance of bacteria, the case was sent for reprocessing and a new slide was prepared from the same specimen sample.

Slides having at least 1,000 cells with evaluable/enumerable signals were deemed sufficient for analysis. Additional factors that were considered when determining if a sample could be analyzed included, slides lacking obscuring contaminants (e.g., inflammation, bacteria, lubricant) and slides having sufficient cells spacing and density.

If a slide was deemed sufficient for analysis, benign analysis of the slide began in the furthest left area of the cell spot and scanning of the slide continued from left to right and top to bottom without overlapping the same cells already viewed.

Applied Spectral Imaging (ASI) GenASIs™ software was used for capture/analysis, analysis/review, and scan/analysis of the slide.

The ASI GenASIs™ software was program to alert the technician when 1,000 cells had been counted.

Nuclei of 1,000 cells were consecutively scored, if each signal was:
1. On or touching the DAPI-stained nucleus,
2. Larger than background spots, and
3. A single spot, a closely-spaced doublet (less than one signal width between), a closely-spaced cluster, or a continuous string.

Cells were not scored if they exhibited the following features:
1. Nuclei that were overlapping such that the signals belonging to each nucleus cannot be clearly distinguished
2. Nuclei that were scratched or physically damaged
3. Nuclei morphologically consistent with non-epithelial cells, such as lymphocytes or neutrophils
4. Nuclei partially covered by fluorescent debris which obscures true signals
5. Nuclei having pale or irregular signals that cannot be distinguished from background
6. Nuclei that did not contain at least one red, one green, one aqua, and one gold signal

Nuclei were scored according to the signal patterns for each probe in the set, such that a normal pattern would contain two signals of each color (2 red, 2 green, 2 aqua, and 2 gold). Nuclei not exhibiting a normal pattern would similarly be scored, enumerating the number of red signals, green signals, aqua signals, and gold signals.

Using the ASI GenASIs™ software, cell counts were classified according to pattern and recorded using the mCounter:
1. Normal (2 red, 2 green, 2 aqua, 2 gold)
2. Tetraploid (4 red, 4 green, 4 aqua, 4 gold)
3. 3q26 Gain (≧3 red, 2 green, 2 aqua, 2 gold)
4. 5p15 Gain (2 red, ≧3 green, 2 aqua, 2 gold)
5. Cen7 Gain (2 red, 2 green, ≧3 aqua, 2 gold)
6. 20q13 Gain (2 red, 2 green, 2 aqua, ≧3 gold)

Multiple Gains (any cell with ≧3 copies of ≧2 probe loci). The exact signal enumeration pattern was recorded on the FISH Manual Score Sheet.

After a minimum of 1,000 epithelial cells were scored and recorded, the remainder of cells in the cell spot were scanned for any additional cells with abnormal signal patterns. If any abnormal cells were found, scoring resumed until an additional 500 cells were scored. After the entire cell spot was scanned, the specimen was determined to be positive or negative for gains of each individual probe according to the established cut-offs.

If less than 1000 evaluable nuclei present and cut-offs for positivity were not reached, the slide was considered uninformative due to insufficient cellularity for evaluation. Analysis of the specimen was then repeated on another slide, if there was remaining specimen for processing.

If there were less than 1,000 evaluable nuclei present and cut-offs for positivity were met, the case could be considered as Positive at the discretion of the signing pathologist and medical director.

Once the scoring was complete, the mCounter in the ASI GenASIs™ software was stopped and the results were saved and approved.

b. Image Acquisition

A minimum of 2 cells were be imaged and saved per case using the ASI GenASIs™ software. Briefly, an image was captured by focusing on a cell of interest with the 60× or 100× oil objective on the DAPI filter using the ASI GenASIs™ software and a camera. Images for each signal (e.g., red, green, aqua, and gold layers) were captured by turning the filter wheel.

After all layers were captured separately, the layers were combined to show each individual layer using the FISHView® application in the ASI GenASIs™ software. After combining the layers, each layer was adjusted to take out any background noise or bring up true signal intensity using the software.

After all the desired cells of interest were imaged, specific cell images were tagged to be used when reporting the data. Reports were then created using the ASI GenASIs™ software. The report was saved as a PDF file and also printed for recordkeeping.

Cases were archived on a quarterly basis or earlier when deemed necessary. Cases were archived using the ASI GenASIs™ software.

Image files and case reports were retained for the appropriate period of time. In some cases, the image files are stored for at least 10 years.

### Example 3

### HPV 4C FISH—Automated Scoring

Slides were prepared from cervical or vaginal ThinPrep Pap Test specimen according to the preparation and hybridization protocol discussed in Example 1 and were stored at −20° C. until they were ready to be analyzed with the following procedure.

Automated scoring of cells was accomplished using the BioView Duet Scanning System and a Solo™ Workstation using the equipment and software provided by the manufacturer (BioView, Inc., Billerica, Mass., USA). The software was also programmed to stop counting after detecting 1500 normal cells.

The slides were then scanned using the system and software provided.

After a slide finished scanning, analysis of a sample was performed offline using the software provided by the system. The data obtained by the software was reviewed to confirm that the BioView software classified the cells appropriately. If the BioView software accurately classified the cells, then the data was not changed. However, a cell not classified correctly was reclassified by selecting the cell and specifying the correct number of classification in the software (i.e. “1” for normal, “2” for abnormal, etc.).

Images were enhanced and processed for exporting using the software provided. At least 2 images were sent to the report for record keeping.

Analysis continued systematically by reviewing all cells in each classification. Cells marked as Unclassified were not necessarily reviewed. After all cells were reviewed, the final distribution of cells in each classification was analyzed. The results for each classification could be reviewed and analyzed using the software provided. Reports could also be generated, saved, and/or printed using the software. Copies of the case report, including at least 2 images, were be saved to be retained for every case for at least 10 years.

After analysis was completed with Automated (BioView) scanning, a manual review of the slide was performed. The manual review was a quick scan of the entire cell spot to ensure that there are no abnormal cells were present in a significant amount that would change a Negative result to a Positive result. If cells with abnormal cells were found, then the slide was re-run on BioView and re-analyzed to determine if the abnormal cells were captured. If the software continued to report the sample as being negative, the slide was reviewed manually to confirm the appropriate result. Results of automated vs. manually scored cases were recorded.

Any case that failed to scan on BioView was sent for manual scoring. A case that scanned on BioView but did not include at least 1,000 cells after analysis was re-ran on BioView with an increase in the stop criteria. If, after re-running the sample, less than 1,000 cells were still analyzed, then the sample was sent for manual scoring.

Cases classified at or near the established cut off for positivity for multiple gains were sent for manual scoring for confirmation of the positive result.

After analysis was completed by a trained technologist, the case report was sent to a pathologist. The pathologist reviewed cell images on the BioView Solo Workstation and was able to review the slide on the manual FISH microscope as needed.

### Example 4

### HPV 4C FISH—Cut-Off Thresholds

a. Cut-Off Threshold Criteria

Cut-off thresholds were established in validation studies to determine the number of cells of abnormal pattern (e.g., a gain in one or more biomarker/probe being analyzed) that may be found in clinically normal patients routinely evaluated using the methods disclosed herein. The cut-off value for each abnormal pattern represents the minimal percentage of cells within a sample being analyzed that must show a gain in that abnormal pattern to be able classify the entire sample as “abnormal” or “positive” for reporting purposes.

Cut-off thresholds were based on validation studies that evaluated multiple specimen samples obtained from patients otherwise considered to be clinically normal and had a “negative specimen” based on (1) cytologically negative results (diagnosed NILM, or Negative for Intraepithelial Lesion or Malignancy) and (2) results negative for high-risk HPV (types 16, 18, 31, 33, 35, 39, 45, 51, 52, 56, 58, 59, 68). Although each complete specimen sample was considered a “negative specimen” based on the preceding criteria, individual cells within the samples were found to be abnormal (or false positive) based on gains in 3q, 5p, CEP7, and/or 20q, as detected by FISH. Accordingly, these negative specimen were analyzed and cut-off values were determined for each individual abnormality (e.g., a gain in only one of 3q, 5p, CEP7, or 20q) and multiple abnormalities (a gain in more than one of 3q, 5p, CEP7, and/or 20q).

Specifically, the validation studies were conducted by evaluating a minimum of 1,000 cells in each specimen to determine the presence and amount of each of the following probes: 3q26 (red), 5p15 (green), CEP7 (aqua), and 20q13 (gold) (Cancer Genetics, Inc., Rutherford, N.J., USA). After analysis, cells were categorized as either normal cells or abnormal cells based on the following critera:
1. Normal—a single cell that contained exactly two (2) copies of each probe (e.g., exactly two copies of 3q, 5p, CEP7, and 20q) (e.g., FIGS. 1a and 1b).
2. Single gain—a single cell that contained three (3) or more copies of one probe and exactly two (2) copies of the other probes (e.g., three copies of 3q and exactly two copies of 5p, CEP7, and 20q).
3. Multiple gains—a single cell that contained three (3) or more copies of more than one probe (e.g., gains in 3q and 5p; or gains in 3q and 20q; or gains in 3q and 5p and 20q; or gains in 3q and 5p and CEP7; etc.). The multiple gains category did not include cells that were tetraploid (e.g., FIGS. 2a and 2b).
4. Tetraploid—cells that contained exactly four (4) copies of each probe. A cell that appeared to be generally tetraploid (4 copies of most chromosomes) that also had an abnormal, non-tetraploid gain in one of the probes evaluated was categorized under the “multiple gains” category. For example, a cell having four (4) copies of 3q, 5p, and 20q but three (3) copies of CEP7, would be classified as a multiple gains result. Similarly, a cell having four (4) copies of 3q, 5p, and 20q, but two (2) copies of CEP7 would also be considered as a multiple gain.

Cut-off values for gains in each probe (and combinations of multiple gains) were calculated from the data after all of the samples in the validation studies were analyzed, categorized, and quantified. Specifically, the cut-off values were determined by calculating the BETAINV from the data obtained for each category of analyzed samples using the following formula:

Cut-Off=(BETAINV(p,α,β))*100
p=confidence level
α=(C %+1)
β=the number cells considered for a specimen sample (i.e., 1,000),

where the value for “C %” used for (α) represents the percentage of the cell sample having the highest percentage of gain in the abnormality analyzed (category (1) to (4) above)), normalized to a cell sample size of 1,000. That is, C % used to determine the cut-off values is taken from the sample having the highest percentage of abnormalities as calculated from: C %=(# cells detected having a gain in the abnormality analyzed÷# total cells analyzed in the sample)×1,000.

The CEILING for each cut-off value was also calculated to round the cut-off values up to the next 0.1%. This calculation was performed to account for small variations in the samples, detection, and or data that might affect the final significant figure.

As discussed above, the cut-off value for each abnormal pattern represents the minimal percentage of cells within a sample that must show a gain in that abnormal pattern to be able classify the entire sample as “abnormal” or “positive” for reporting purposes. Thus, a sample analyzed using the methods disclosed herein will be classified as “normal” or “negative” for reporting purposes if the sample does not have a gain in any abnormality above the cut-off value calculated in these studies. Conversely, a sample analyzed using the methods disclosed herein will be classified as “abnormal” or “positive” for reporting purposes if the sample has a gain in any abnormality above the cut-off value calculated in these studies.

b. Cut-Off Values—Theoretical

Theoretical cut-off values were calculated based on the minimum number of abnormalities that can be present in a sample. Specifically, theoretical values were obtained based on the assumption that any abnormal cell that is present in a sample is indicative of a positive result.

Accordingly, the theoretical cut-off values were calculated using the BETAINV function discussed above, where α=1 (i.e., 0%+1). The theoretical cut-off values obtained in this study are shown in Table 1.

c. Cut-Off Values—Manual Scoring

Sixty-three (63) samples of cervical cells were obtained from clinically normal patients and processed according to the methods described in Example 1. The cells of these samples were then analyzed by FISH and manually scored, as described in Example 2.

The cells analyzed within each sample were categorized as (1) normal; (2) single gain; (3) multiple gains; and (4) tetraploid, as discussed above. The results of the FISH analysis for these samples are shown in Table 2.

The cut-off threshold values calculated for each category of abnormal cells, with confidence levels ranging from 90% to 99% in single digit increments, are shown in Table 3.

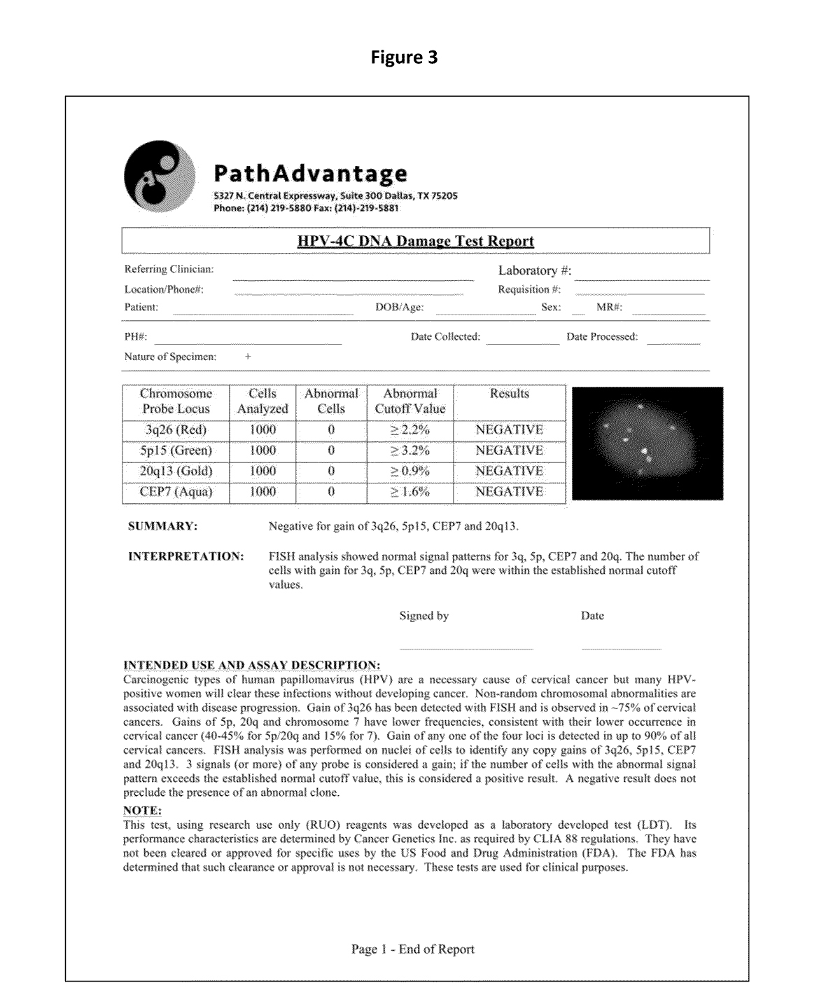

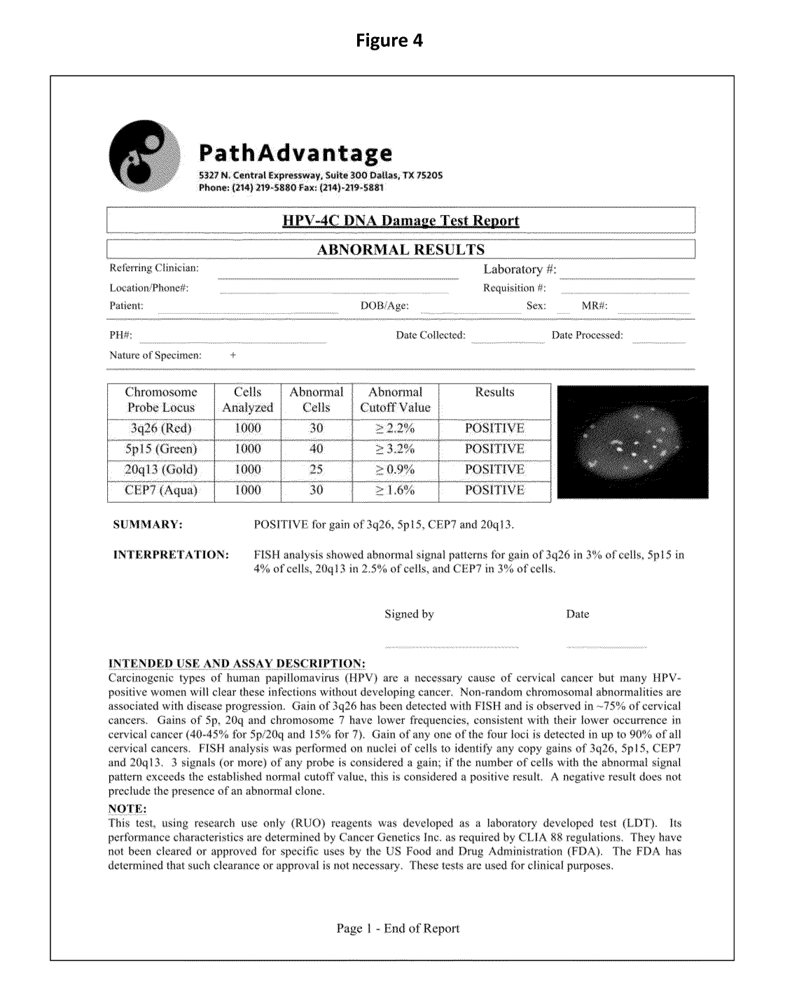

The data and results obtained from this validation study provide cut-off values for each abnormality tested using the manual scoring methods disclosed herein. These cut-off values were then used to assess whether a sample was classified and reported as “negative” (FIG. 3) or “positive” (FIG. 4) for the presence of abnormalities.

d. Cut-Off Values—Automated Scoring

Seventy-three (73) samples of cervical cells were obtained from clinically normal patients and processed according to the methods described in Example 1. The cells of these samples were then analyzed by FISH and scored by the automated method described in Example 3.

The cells analyzed within each sample were categorized as (1) normal; (2) single gain; (3) multiple gains; and (4) tetraploid, as discussed above. The results of the FISH analysis for these samples are shown in Table 4.

The cut-off threshold values calculated for each category of abnormal cells, with confidence levels ranging from 90% to 99% in single digit increments, are shown in Table 5.

The data and results obtained from this validation study provide cut-off values for each abnormality based the automated scoring method disclosed herein. These cut-off values were then used to assess whether a sample was classified and reported as “negative” (FIG. 3) or “positive” (FIG. 4) for the presence of abnormalities.

TABLE 1
Theoretical Cut-Off Values

| | Confidence | CUT-OFF THRESHOLD |
|---|---|---|
| | (p) | (gains normalized %) |
| BETAINV | 99% | 0.46 |
| | 98% | 0.39 |
| | 97% | 0.35 |
| | 96% | 0.32 |
| | 95% | 0.30 |
| | 94% | 0.28 |
| | 93% | 0.27 |
| | 92% | 0.25 |
| | 91% | 0.24 |
| | 90% | 0.23 |
| CEILING(BETAINV) | 99% | 0.5 |
| | 98% | 0.4 |
| | 97% | 0.4 |
| | 96% | 0.4 |
| | 95% | 0.3 |
| | 94% | 0.3 |
| | 93% | 0.3 |
| | 92% | 0.3 |
| | 91% | 0.3 |
| | 90% | 0.3 |

TABLE 2
Abnormalities observed by FISH in sixty-three (63) negative specimen
samples by manual scoring

| | | | GAINS (normalized %) ¹ | | | | | |
|---|---|---|---|---|---|---|---|---|
| Sample | Total Cells | Normal | 3q | 5p | CEP 7 | 20q | | |
| # | Counted | Cells | (Red) | (Green) | (Aqua) | (Gold) | Tetraploid | Multiple |
| 1 | 1009 | 1006 | 1 | 0 | 1 | 0 | 1 | 0 |
| 2 | 1001 | 997 | 0 | 1 | 1 | 0 | 2 | 0 |
| 3 | 1006 | 995 | 2 | 1 | 2 | 0 | 1 | 5 |
| 4 | 1004 | 1004 | 0 | 0 | 0 | 0 | 0 | 0 |
| 5 | 1005 | 1004 | 0 | 0 | 0 | 1 | 0 | 0 |
| 6 | 1000 | 999 | 0 | 0 | 1 | 0 | 0 | 0 |
| 7 | 1000 | 999 | 0 | 0 | 0 | 0 | 1 | 0 |
| 8 | 1002 | 998 | 1 | 0 | 2 | 0 | 1 | 0 |
| 9 | 1000 | 996 | 2 | 0 | 0 | 0 | 2 | 0 |
| 10 | 1001 | 994 | 0 | 1 | 0 | 2 | 3 | 1 |
| 11 | 1006 | 1003 | 0 | 2 | 0 | 1 | 0 | 0 |
| 13 | 1000 | 999 | 0 | 0 | 0 | 1 | 0 | 0 |
| 15 | 1000 | 999 | 0 | 0 | 0 | 0 | 1 | 0 |
| 16 | 1000 | 1000 | 0 | 0 | 0 | 0 | 0 | 0 |
| 17 | 1000 | 999 | 0 | 0 | 0 | 0 | 1 | 0 |
| 18 | 1001 | 999 | 0 | 0 | 0 | 1 | 0 | 0 |
| 19 | 1000 | 995 | 5 | 0 | 0 | 0 | 0 | 0 |
| 20 | 1001 | 998 | 0 | 2 | 0 | 0 | 1 | 0 |
| 21 | 1000 | 999 | 1 | 0 | 0 | 0 | 0 | 0 |
| 22 | 1000 | 997 | 0 | 0 | 0 | 3 | 0 | 0 |
| 23 | 1000 | 993 | 1 | 0 | 0 | 0 | 6 | 0 |
| 25 | 1000 | 997 | 1 | 0 | 0 | 0 | 2 | 0 |
| 27 | 1000 | 992 | 4 | 0 | 1 | 2 | 0 | 1 |
| 28 | 1000 | 997 | 1 | 0 | 0 | 0 | 2 | 0 |
| 29 | 1000 | 1000 | 0 | 0 | 0 | 0 | 0 | 0 |
| 30 | 1000 | 1000 | 0 | 0 | 0 | 0 | 0 | 0 |
| 31 | 1000 | 998 | 1 | 0 | 0 | 0 | 1 | 0 |
| 32 | 1000 | 1000 | 0 | 0 | 0 | 0 | 0 | 0 |
| 33 | 1000 | 994 | 3 | 3 | 0 | 0 | 0 | 0 |
| 36 | 1000 | 1000 | 0 | 0 | 0 | 0 | 0 | 0 |
| 37 | 1000 | 1000 | 0 | 0 | 0 | 0 | 0 | 0 |
| 38 | 1000 | 1000 | 0 | 0 | 0 | 0 | 0 | 0 |
| 39 | 1000 | 1000 | 0 | 0 | 0 | 0 | 0 | 0 |
| 40 | 1000 | 999 | 1 | 0 | 0 | 0 | 0 | 0 |
| 41 | 1000 | 996 | 3 | 1 | 0 | 0 | 0 | 0 |
| 42 | 1000 | 999 | 1 | 0 | 0 | 0 | 0 | 0 |
| 43 | 1000 | 1000 | 0 | 0 | 0 | 0 | 0 | 0 |
| 44 | 1000 | 998 | 0 | 0 | 0 | 2 | 0 | 0 |
| 45 | 1000 | 1000 | 0 | 0 | 0 | 0 | 0 | 0 |
| 46 | 1000 | 1000 | 0 | 0 | 0 | 0 | 0 | 0 |
| 47 | 1000 | 995 | 1 | 0 | 0 | 1 | 3 | 0 |
| 48 | 1000 | 1000 | 0 | 0 | 0 | 0 | 0 | 0 |
| 49 | 1000 | 998 | 1 | 0 | 0 | 0 | 1 | 0 |
| 76 | 1000 | 993 | 0 | 1 | 0 | 0 | 4 | 2 |
| 78 | 1000 | 999 | 0 | 0 | 0 | 0 | 1 | 0 |
| 79 | 1000 | 996 | 0 | 0 | 1 | 0 | 2 | 1 |
| 81 | 1000 | 998 | 1 | 1 | 0 | 0 | 0 | 0 |
| 82 | 1000 | 999 | 0 | 1 | 0 | 0 | 0 | 0 |
| 83 | 1000 | 992 | 1 | 4 | 3 | 0 | 0 | 0 |
| 84 | 1000 | 992 | 0 | 1 | 2 | 2 | 2 | 1 |
| 85 | 1000 | 998 | 0 | 0 | 0 | 1 | 0 | 1 |
| 86 | 1000 | 997 | 2 | 0 | 0 | 0 | 1 | 0 |
| 87 | 1000 | 1000 | 0 | 0 | 0 | 0 | 0 | 0 |
| 88 | 1000 | 997 | 1 | 0 | 2 | 0 | 0 | 0 |
| 89 | 1000 | 998 | 1 | 1 | 0 | 0 | 0 | 0 |
| 90 | 1000 | 997 | 3 | 0 | 0 | 0 | 0 | 0 |
| 91 | 1000 | 995 | 1 | 1 | 2 | 1 | 0 | 0 |
| 94 | 1000 | 993 | 2 | 0 | 0 | 1 | 3 | 1 |
| 95 | 1000 | 999 | 0 | 0 | 1 | 0 | 0 | 0 |
| 97 | 1000 | 997 | 3 | 0 | 0 | 0 | 0 | 0 |
| 98 | 1000 | 995 | 1 | 0 | 0 | 2 | 1 | 1 |
| 99 | 1000 | 999 | 0 | 0 | 0 | 1 | 0 | 0 |
| 100 | 1000 | 1000 | 0 | 0 | 0 | 0 | 0 | 0 |

¹ Gains percentages were normalized to a cell sample of 1000 to account for differences in the total number of cells counted in each sample using the following formula: normalized % = (# cells with a gain ÷ # total cells counted) × 1,000

TABLE 3
Cut-Off thresholds determined from manually scored samples

| | | CUT-OFF THRESHOLDS (gains normalized %) | | | | | |
|---|---|---|---|---|---|---|---|
| | Confidence (p) | 3q ³ | 5p ⁴ | CEP 7 ⁵ | 20q ⁶ | Tetraploid ⁷ | Multiple ⁸ |
| (BETAINV) ¹ | 99% | 1.30 | 1.15 | 1.00 | 1.00 | 1.44 | 1.30 |
| | 98% | 1.19 | 1.05 | 0.90 | 0.90 | 1.33 | 1.19 |
| | 97% | 1.13 | 0.99 | 0.85 | 0.85 | 1.26 | 1.13 |
| | 96% | 1.08 | 0.94 | 0.80 | 0.80 | 1.21 | 1.08 |
| | 95% | 1.04 | 0.91 | 0.77 | 0.77 | 1.17 | 1.04 |
| | 94% | 1.01 | 0.88 | 0.74 | 0.74 | 1.14 | 1.01 |
| | 93% | 0.99 | 0.85 | 0.72 | 0.72 | 1.11 | 0.99 |
| | 92% | 0.96 | 0.83 | 0.70 | 0.70 | 1.09 | 0.96 |
| | 91% | 0.94 | 0.81 | 0.68 | 0.68 | 1.06 | 0.94 |
| | 90% | 0.92 | 0.79 | 0.66 | 0.66 | 1.04 | 0.92 |
| CEILING(BETAINV) ² | 99% | 1.3 | 1.2 | 1.0 | 1.0 | 1.5 | 1.3 |
| | 98% | 1.2 | 1.1 | 1.0 | 1.0 | 1.4 | 1.2 |
| | 97% | 1.2 | 1.0 | 0.9 | 0.9 | 1.3 | 1.2 |
| | 96% | 1.1 | 1.0 | 0.9 | 0.9 | 1.3 | 1.1 |
| | 95% | 1.1 | 1.0 | 0.8 | 0.8 | 1.2 | 1.1 |
| | 94% | 1.1 | 0.9 | 0.8 | 0.8 | 1.2 | 1.1 |
| | 93% | 1.0 | 0.9 | 0.8 | 0.8 | 1.2 | 1.0 |
| | 92% | 1.0 | 0.9 | 0.7 | 0.7 | 1.1 | 1.0 |
| | 91% | 1.0 | 0.9 | 0.7 | 0.7 | 1.1 | 1.0 |
| | 90% | 1.0 | 0.8 | 0.7 | 0.7 | 1.1 | 1.0 |

¹ Cut-Off = (BETAINV(p, α, β))*100
p = confidence level
α = (C % + 1)
β = the number cells considered for a specimen sample (i.e., 1,000), where the value for “C %” used for calculating (α) represents the percentage of the cell sample having the highest percentage of gain in the abnormality analyzed (category (1) to (4) above)), normalized to a cell sample size of 1,000. That is, C % used to determine the cut-off values is taken from the sample having the highest percentage of abnormalities as calculated from: C % = (# cells detected having a gain in the abnormality analyzed ÷ # total cells analyzed in the sample) × 1,000.
² Cut-Off = CEILING(BETAINV), where “ceiling” is the number rounded up, away from zero, to the nearest multiple of significance.
³ α for 3q gains = 6 (Sample 19, Table 1)
⁴ α for 5p gains = 5 (Sample 83, Table 1)
⁵ α for CEP7 gains = 4 (Sample 83, Table 1)
⁶ α for 20q gains = 4 (Sample 22, Table 1)
⁷ α for Tetraploid = 7 (Sample 23, Table 1)
⁸ α for Multiple gains = 6 (Sample 3, Table 1)

TABLE 4
Abnormalities observed by FISH in seventy-three (73) negative specimen
samples by automated scoring

| | | | GAINS (normalized %) ¹ | | | | | |
|---|---|---|---|---|---|---|---|---|
| Sample | Total Cells | Normal | 3q | 5p | CEP 7 | 20q | | |
| # | Counted | Cells | (Red) | (Green) | (Aqua) | (Yellow) | Tetraploid | Multiple |
| 27 | 1007 | 999 | 6.0 | 0.0 | 1.0 | 0.0 | 1.0 | 0.0 |
| 28 | 1223 | 1222 | 0.8 | 0.0 | 0.0 | 0.0 | 0.0 | 0.0 |
| 29 | 1228 | 1225 | 2.4 | 0.0 | 0.0 | 0.0 | 0.0 | 0.0 |
| 30 | 1297 | 1296 | 0.0 | 0.8 | 0.0 | 0.0 | 0.0 | 0.0 |
| 31 | 1367 | 1358 | 0.7 | 4.4 | 0.7 | 0.0 | 0.7 | 0.0 |
| 32 | 1116 | 1110 | 3.6 | 0.9 | 0.9 | 0.0 | 0.0 | 0.0 |
| 33 | 1205 | 1201 | 0.8 | 1.7 | 0.0 | 0.0 | 0.8 | 0.0 |
| 36 | 1096 | 1096 | 0.0 | 0.0 | 0.0 | 0.0 | 0.0 | 0.0 |
| 37 | 1225 | 1223 | 0.0 | 1.6 | 0.0 | 0.0 | 0.0 | 0.0 |
| 38 | 1531 | 1527 | 0.7 | 0.0 | 0.0 | 0.0 | 2.0 | 0.0 |
| 39 | 1170 | 1168 | 1.7 | 0.0 | 0.0 | 0.0 | 0.0 | 0.0 |
| 40 | 1307 | 1305 | 0.0 | 0.0 | 0.8 | 0.0 | 0.8 | 0.0 |
| 41 | 1088 | 1074 | 11.0 | 0.0 | 0.0 | 0.0 | 0.0 | 1.8 |
| 42 | 1256 | 1244 | 1.6 | 2.4 | 1.6 | 0.0 | 4.0 | 0.0 |
| 43 | 1324 | 1320 | 0.0 | 0.8 | 1.5 | 0.0 | 0.8 | 0.0 |
| 44 | 1177 | 1177 | 0.0 | 0.0 | 0.0 | 0.0 | 0.0 | 0.0 |
| 45 | 1102 | 1100 | 0.0 | 1.8 | 0.0 | 0.0 | 0.0 | 0.0 |
| 46 | 1263 | 1263 | 0.0 | 0.0 | 0.0 | 0.0 | 0.0 | 0.0 |
| 47 | 1023 | 1022 | 0.0 | 0.0 | 1.0 | 0.0 | 0.0 | 0.0 |
| 48 | 1322 | 1316 | 1.5 | 1.5 | 0.8 | 0.8 | 0.0 | 0.0 |
| 49 | 1180 | 1180 | 0.0 | 0.0 | 0.0 | 0.0 | 0.0 | 0.0 |
| 50 | 1078 | 1077 | 0.0 | 0.0 | 0.0 | 0.0 | 0.9 | 0.0 |
| 51 | 1491 | 1484 | 2.0 | 0.0 | 1.3 | 0.0 | 1.3 | 0.0 |
| 52 | 1019 | 1019 | 0.0 | 0.0 | 0.0 | 0.0 | 0.0 | 0.0 |
| 53 | 1249 | 1244 | 0.0 | 0.8 | 3.2 | 0.0 | 0.0 | 0.0 |
| 54 | 1349 | 1335 | 0.0 | 0.7 | 4.4 | 0.0 | 3.7 | 1.5 |
| 55 | 1479 | 1473 | 1.4 | 1.4 | 0.0 | 0.0 | 1.4 | 0.0 |
| 56 | 1335 | 1328 | 1.5 | 2.2 | 0.0 | 0.0 | 0.7 | 0.7 |
| 57 | 1009 | 1006 | 3.0 | 0.0 | 0.0 | 0.0 | 0.0 | 0.0 |
| 58 | 1370 | 1361 | 0.7 | 0.7 | 0.0 | 1.5 | 0.7 | 2.9 |
| 59 | 1544 | 1530 | 1.9 | 1.3 | 5.2 | 0.6 | 0.0 | 0.0 |
| 60 | 1490 | 1478 | 0.0 | 5.4 | 0.7 | 0.0 | 2.0 | 0.0 |
| 61 | 1309 | 1307 | 0.0 | 0.8 | 0.0 | 0.0 | 0.8 | 0.0 |
| 62 | 1359 | 1349 | 0.0 | 2.2 | 0.0 | 0.0 | 2.9 | 2.2 |
| 63 | 1442 | 1428 | 0.0 | 2.1 | 5.5 | 0.7 | 0.0 | 1.4 |
| 64 | 1409 | 1400 | 0.7 | 2.1 | 0.0 | 0.7 | 0.7 | 2.1 |
| 66 | 1437 | 1427 | 2.8 | 0.7 | 0.0 | 0.7 | 1.4 | 1.4 |
| 67 | 1486 | 1454 | 0.7 | 19.5 | 0.7 | 0.0 | 0.7 | 0.0 |
| 68 | 1546 | 1543 | 0.0 | 0.6 | 0.6 | 0.6 | 0.0 | 0.0 |
| 69 | 1445 | 1441 | 0.7 | 1.4 | 0.0 | 0.7 | 0.0 | 0.0 |
| 70 | 1164 | 1161 | 0.0 | 0.0 | 0.9 | 0.0 | 1.7 | 0.0 |
| 71 | 1408 | 1407 | 0.0 | 0.0 | 0.7 | 0.0 | 0.0 | 0.0 |
| 72 | 1206 | 1203 | 0.8 | 1.7 | 0.0 | 0.0 | 0.0 | 0.0 |
| 73 | 1545 | 1537 | 0.0 | 2.6 | 2.6 | 0.0 | 0.0 | 0.0 |
| 74 | 1034 | 1027 | 0.0 | 1.9 | 3.9 | 0.0 | 0.0 | 1.0 |
| 75 | 1625 | 1624 | 0.0 | 0.6 | 0.0 | 0.0 | 0.0 | 0.0 |
| 76 | 1488 | 1468 | 3.4 | 0.7 | 6.7 | 0.0 | 0.7 | 2.0 |
| 78 | 1442 | 1437 | 0.7 | 2.1 | 0.0 | 0.0 | 0.7 | 0.0 |
| 79 | 1421 | 1413 | 0.0 | 0.0 | 3.5 | 0.7 | 0.0 | 1.4 |
| 81 | 1328 | 1318 | 3.0 | 3.0 | 0.8 | 0.8 | 0.0 | 0.0 |
| 82 | 1326 | 1324 | 0.8 | 0.0 | 0.8 | 0.0 | 0.0 | 0.0 |
| 83 | 1054 | 1043 | 2.8 | 3.8 | 2.8 | 0.0 | 0.9 | 0.0 |
| 84 | 1267 | 1257 | 0.0 | 3.2 | 4.7 | 0.0 | 0.0 | 0.0 |
| 85 | 1044 | 1037 | 2.9 | 1.9 | 0.0 | 0.0 | 1.0 | 1.0 |
| 86 | 1494 | 1487 | 1.3 | 0.0 | 2.7 | 0.0 | 0.7 | 0.0 |
| 87 | 1479 | 1470 | 2.7 | 1.4 | 0.7 | 0.0 | 0.7 | 0.7 |
| 88 | 1478 | 1470 | 0.0 | 0.7 | 1.4 | 2.0 | 0.0 | 1.4 |
| 89 | 1191 | 1185 | 0.0 | 4.2 | 0.8 | 0.0 | 0.0 | 0.0 |
| 90 | 1266 | 1254 | 7.9 | 1.6 | 0.0 | 0.0 | 0.0 | 0.0 |
| 91 | 1265 | 1245 | 6.3 | 3.2 | 5.5 | 0.0 | 0.0 | 0.8 |
| 95 | 1452 | 1451 | 0.0 | 0.7 | 0.0 | 0.0 | 0.0 | 0.0 |
| 97 | 1486 | 1460 | 7.4 | 0.7 | 4.7 | 0.7 | 0.0 | 0.0 |
| 98 | 1398 | 1391 | 3.6 | 0.0 | 0.0 | 1.4 | 0.0 | 0.0 |
| 99 | 1610 | 1580 | 8.1 | 6.8 | 3.1 | 0.0 | 0.6 | 0.0 |
| 100 | 1166 | 1163 | 1.7 | 0.0 | 0.0 | 0.0 | 0.9 | 0.0 |
| 103 | 1456 | 1453 | 0.7 | 0.7 | 0.0 | 0.7 | 0.0 | 0.0 |
| 105 | 1469 | 1463 | 2.7 | 0.0 | 0.0 | 0.7 | 0.7 | 0.0 |
| 108 | 1700 | 1665 | 11.2 | 1.2 | 2.9 | 1.2 | 0.0 | 1.2 |
| 109 | 1490 | 1458 | 8.1 | 2.7 | 6.0 | 1.3 | 0.7 | 1.3 |
| 110 | 1442 | 1417 | 4.2 | 2.8 | 3.5 | 0.0 | 1.4 | 0.7 |
| 111 | 1298 | 1294 | 2.3 | 0.8 | 0.0 | 0.0 | 0.0 | 0.0 |
| 112 | 1568 | 1538 | 9.6 | 3.8 | 0.6 | 0.6 | 0.6 | 0.6 |
| 113 | 1292 | 1287 | 1.5 | 0.0 | 0.8 | 0.0 | 0.0 | 1.5 |

¹ Gains percentages were normalized to a cell sample of 1000 to account for differences in the total number of cells counted in each sample using the following formula: normalized % = (# cells with a gain ÷ # total cells counted) × 1,000

TABLE 5
Cut-Off thresholds determined from automated samples

| | | CUT-OFF THRESHOLDS (gains normalized %) | | | | | |
|---|---|---|---|---|---|---|---|
| | Confidence (p) | 3q | 5p | CEP7 | 20q | Tetra | Multiple |
| BETAINV | 99% | 2.1 | 3.2 | 1.5 | 0.8 | 1.2 | 1.0 |
| | 98% | 2.0 | 3.0 | 1.4 | 0.7 | 1.1 | 0.9 |
| | 97% | 1.9 | 2.9 | 1.4 | 0.7 | 1.0 | 0.8 |
| | 96% | 1.9 | 2.8 | 1.3 | 0.7 | 0.9 | 0.8 |
| | 95% | 1.8 | 2.8 | 1.3 | 0.6 | 0.9 | 0.8 |
| | 94% | 1.8 | 2.7 | 1.2 | 0.6 | 0.9 | 0.7 |
| | 93% | 1.7 | 2.7 | 1.2 | 0.6 | 0.9 | 0.7 |
| | 92% | 1.7 | 2.7 | 1.2 | 0.6 | 0.8 | 0.7 |
| | 91% | 1.7 | 2.6 | 1.2 | 0.5 | 0.8 | 0.7 |
| | 90% | 1.7 | 2.6 | 1.1 | 0.5 | 0.8 | 0.7 |
| CEILING(BETAINV) | 99% | 2.2 | 3.2 | 1.6 | 0.9 | 1.2 | 1.0 |
| | 98% | 2.1 | 3.1 | 1.5 | 0.8 | 1.1 | 0.9 |
| | 97% | 2.0 | 3.0 | 1.4 | 0.7 | 1.0 | 0.9 |
| | 96% | 1.9 | 2.9 | 1.4 | 0.7 | 1.0 | 0.8 |
| | 95% | 1.9 | 2.8 | 1.3 | 0.7 | 1.0 | 0.8 |
| | 94% | 1.8 | 2.8 | 1.3 | 0.7 | 0.9 | 0.8 |
| | 93% | 1.8 | 2.7 | 1.3 | 0.6 | 0.9 | 0.8 |
| | 92% | 1.8 | 2.7 | 1.2 | 0.6 | 0.9 | 0.7 |
| | 91% | 1.7 | 2.7 | 1.2 | 0.6 | 0.9 | 0.7 |
| | 90% | 1.7 | 2.6 | 1.2 | 0.6 | 0.8 | 0.7 |

¹ Cut-Off = (BETAINV(p, α, β))*100
p = confidence level
α = (C % + 1)
β = the number cells considered for a specimen sample (i.e., 1,000), where the value for “C %” used for calculating (α) represents the percentage of the cell sample having the highest percentage of gain in the abnormality analyzed (category (1) to (4) above)), normalized to a cell sample size of 1,000. That is, C % used to determine the cut-off values is taken from the sample having the highest percentage of abnormalities as calculated from: C % = (# cells detected having a gain in the abnormality analyzed ÷ # total cells analyzed in the sample) × 1,000.
² Cut-Off = CEILING(BETAINV), where “ceiling” is the number rounded up, away from zero, to the nearest multiple of significance.
³ α for 3q gains = 12.2 (Sample 108, Table 3)
⁴ α for 5p gains = 19.5 (Sample 67, Table 3)
⁵ α for CEP7 gains = 6.7 (Sample 76, Table 3)
⁶ α for 20q gains = 2.0 (Sample 88, Table 3)
⁷ α for Tetraploid = 4.0 (Sample 42, Table 3)
⁸ α for Multiple gains = 2.9 (Sample 58, Table 3)

TABLE 6
List of cancer genes on human chromosome 3*

| | GoldenPath | | |
|---|---|---|---|
| Symbol | (Mb) | Location | Description |
| SCHIP1 | 158991.036 | 3 | schwarmomin interacting protein 1 |
| MIR570 | 195426.272 | 3 | microRNA 570 |
| MYL3 | 46899.357 | 3p | myosin, light chain 3, alkali; ventricular, skeletal, slow |
| POU1F1 | 87308.783 | 3p11.2 | POU class 1 homeobox 1 |
| EPHA3 | 89156.674 | 3p11.2 | EPH receptor A3 |
| CGGBP1 | 88101.1 | 3p12-p11.1 | CGG triplet repeat binding protein 1 |
| NFKBIZ | 101546.834 | 3p12-q12 | nuclear factor of kappa light polypeptide gene enhancer in B-cells |
| | | | inhibitor, zeta |
| VGLL3 | 86987.123 | 3p12.1 | vestigial-like family member 3 |
| CADM2 | 85008.133 | 3p12.2 | cell adhesion molecule 2 |
| CNTN3 | 74311.722 | 3p12.3 | contactin 3 (plasmacytoma associated) |
| ROBO2 | 77147.163 | 3p12.3 | roundabout, axon guidance receptor, homolog 2 (Drosophila) |
| ROBO1 | 78646.388 | 3p12.3 | roundabout, axon guidance receptor, homolog 1 (Drosophila) |
| SHQ1 | 72798.428 | 3p13 | SHQ1, H/ACA ribonucleoprotein assembly factor |
| TXNRD3 | 126325.895 | 3p13-q13.33 | thioredoxin reductase 3 |
| MGLL | 127407.905 | 3p13-q13.33 | monoglyceride lipase |
| LRIG1 | 66429.221 | 3p14 | leucine-rich repeats and immunoglobulin-like domains 1 |
| ARL6IP5 | 69134.09 | 3p14 | ADP-ribosylation factor-like 6 interacting protein 5 |
| EIF4E3 | 71728.44 | 3p14 | eukaryotic translation initiation factor 4E family member 3 |
| ADAMTS9 | 64501.331 | 3p14.1 | ADAM metallopeptidase with thrombospondin type 1 motif, 9 |
| MAGI1 | 65339.906 | 3p14.1 | membrane associated guanylate kinase, WW and PDZ domain |
| | | | containing 1 |
| FAM19A1 | 68040.734 | 3p14.1 | family with sequence similarity 19 (chemokine (C-C motif)-like), |
| | | | member A1 |
| FOXP1 | 71247.034 | 3p14.1 | forkheadbox P1 |
| PDZRN3 | 73431.652 | 3p14.1 | PDZ domain containing ring finger 3 |
| MITF | 69985.751 | 3p14.1-p12.3 | microphthalmia-associated transcription factor |
| FAM107A | 58549.839 | 3p14.2 | family with sequence similarity 107, member A |
| C3orf67 | 58727.737 | 3p14.2 | chromosome 3 open reading frame 67 |
| FHIT | 59735.036 | 3p14.2 | fragile histidine triad |
| NPCDR1 | 59956.576 | 3p14.2 | nasopharyngeal carcinoma, down-regulated 1 |
| SLC25A26 | 66271.168 | 3p14.2 | solute carrier family 25 (S-adenosylmethionine carrier), member 26 |
| RYBP | 72423.744 | 3p14.2 | RING1 and YY1 binding protein |
| TKT | 53259.653 | 3p14.3 | transketolase |
| CACNA1D | 53529.076 | 3p14.3 | calcium channel, voltage-dependent, L type, alpha 1D subunit |
| ESRG | 54666.151 | 3p14.3 | embryonic stem cell related (non-protein coding) |
| ERC2 | 55542.336 | 3p14.3 | ELKS/RAB6-interacting/CAST family member 2 |
| ERC2-IT1 | 55691.243 | 3p14.3 | ERC2 intronic transcript 1 (non-protein coding) |
| ARHGEF3 | 56761.446 | 3p14.3 | Rho guanine nucleotide exchange factor (GEF) 3 |
| HESX1 | 57231.944 | 3p14.3 | HESX homeobox 1 |
| FLNB | 57994.127 | 3p14.3 | filamin B, beta |
| DNASE1L3 | 58178.353 | 3p14.3 | deoxyribonuclease I-like 3 |
| PRICKLE2 | 64079.526 | 3p14.3 | prickle homolog 2 (Drosophila) |
| A4GNT | 137842.56 | 3p14.3 | alpha-1,4-N-acetylglucosaminyltransferase |
| SCN5A | 38589.553 | 3p21 | sodium channel, voltage-gated, type V, alpha subunit |
| CTNNB1 | 41240.942 | 3p21 | catenin (cadherin-associated protein), beta 1, 88 kDa |
| SS18L2 | 42632.298 | 3p21 | synovial sarcoma translocation gene on chromosome 18-like 2 |
| ZNF197 | 44666.511 | 3p21 | zinc finger protein 197 |
| CXCR6 | 45984.973 | 3p21 | chemokine (C-X-C motif) receptor 6 |
| CCR1 | 46243.2 | 3p21 | chemokine (C-C motif) receptor 1 |
| CCR2 | 46395.235 | 3p21 | chemokine (C-C motif) receptor 2 |
| CCR5 | 46411.633 | 3p21 | chemokine (C-C motif) receptor 5 (gene/pseudogene) |
| CCRL2 | 46449.049 | 3p21 | chemokine (C-C motif) receptor-like 2 |
| MAP4 | 47892.18 | 3p21 | microtubule-associated protein 4 |
| CDC25A | 48198.668 | 3p21 | cell division cycle 25A |
| UQCRC1 | 48636.432 | 3p21 | ubiquinol-cytochrome c reductase core protein I |
| NCKIPSD | 48711.272 | 3p21 | NCK interacting protein with SH3 domain |
| ARIH2 | 48956.281 | 3p21 | ariadne RBR E3 ubiquitin protein ligase 2 |
| STGC3 | 49297.518 | 3p21 | uncharacterized STGC3 |
| TCTA | 49449.639 | 3p21 | T-cell leukemia translocation altered |
| DAG1 | 49507.565 | 3p21 | dystroglycan 1 (dystrophin-associated glycoprotein 1) |
| APEH | 49711.435 | 3p21 | acylaminoacyl-peptide hydrolase |
| MST1 | 49721.38 | 3p21 | macrophage stimulating 1 (hepatocyte growth factor-like) |
| UBA7 | 49842.638 | 3p21 | ubiquitin-like modifier activating enzyme 7 |
| MST1R | 49924.436 | 3p21 | macrophage stimulating 1 receptor (c-met-related tyrosine kinase) |
| GNAT1 | 50229.043 | 3p21 | guanine nucleotide binding protein (G protein), alpha transducing |
| | | | activity polypeptide 1 |
| DOCK3 | 50712.672 | 3p21 | dedicator of cytokinesis 3 |
| PCBP4 | 51991.47 | 3p21 | poly(rC) binding protein 4 |
| DUSP7 | 52082.937 | 3p21 | dual specificity phosphatase 7 |
| ALAS1 | 52232.099 | 3p21 | aminolevulinate, delta-, synthase 1 |
| PBRM1 | 52579.368 | 3p21 | polybromo 1 |
| CHDH | 53850.324 | 3p21 | choline dehydrogenase |
| TMF1 | 69068.978 | 3p21-p12 | TATA element modulatory factor 1 |
| WNT5A | 55499.743 | 3p21-p14 | wingless-type MMTV integration site family, member 5A |
| PTPRG | 61547.243 | 3p21-p14 | protein tyrosine phosphatase, receptor type, G |
| COL7A1 | 48601.506 | 3p21.1 | collagen, type VII, alpha 1 |
| MANF | 51422.668 | 3p21.1 | mesencephalic astrocyte-derived neurotrophic factor |
| ABHD14A | 52009.042 | 3p21.1 | abhydrolase domain containing 14A |
| TWF2 | 52262.626 | 3p21.1 | twinfilin actin-binding protein 2 |
| MIRLET7G | 52302.294 | 3p21.1 | microRNA let-7g |
| MIR135A1 | 52328.235 | 3p21.1 | microRNA 135a-1 |
| SEMA3G | 52467.268 | 3p21.1 | sema domain, immunoglobulin domain (Ig), short basic domain, |
| | | | secreted, (semaphorin) 3G |
| NISCH | 52489.524 | 3p21.1 | nischarin |
| GNL3 | 52719.936 | 3p21.1 | guanine nucleotide binding protein-like 3 (nucleolar) |
| GLT8D1 | 52728.5 | 3p21.1 | glycosyltransferase 8 domain containing 1 |
| NEK4 | 52744.796 | 3p21.1 | NIMA-related kinase 4 |
| ITIH1 | 52812.509 | 3p21.1 | inter-alpha-trypsin inhibitor heavy chain 1 |
| ITIH3 | 52828.784 | 3p21.1 | inter-alpha-trypsin inhibitor heavy chain 3 |
| ITIH4 | 52847.006 | 3p21.1 | inter-alpha-trypsin inhibitor heavy chain family, member 4 |
| TMEM110 | 52870.772 | 3p21.1 | transmembrane protein 110 |
| RFT1 | 53122.501 | 3p21.1 | RFT1 homolog (S. cerevisiae) |
| DCP1A | 53317.445 | 3p21.1 | decapping mRNA 1A |
| IL17RB | 53880.577 | 3p21.1 | interleukin 17 receptor B |
| CACNA2D3 | 54156.693 | 3p21.1 | calcium channel, voltage-dependent, alpha 2/delta subunit 3 |
| IL17RD | 57124.01 | 3p21.1 | interleukin 17 receptor D |
| DNAH12 | 57327.727 | 3p21.1 | dynein, axonemal, heavy chain 12 |
| ID2B | 62109.166 | 3p21.1 | inhibitor of DNA binding 2B, dominant negative helix-loop-helix |
| | | | protein (pseudogene) |
| FEZF2 | 62355.347 | 3p21.1 | FEZ family zinc finger 2 |
| CADPS | 62384.021 | 3p21.1 | Ca++-dependent secretion activator |
| PROK2 | 71820.806 | 3p21.1 | prokineticin 2 |
| ATXN7 | 63850.233 | 3p21.1-p12 | ataxin 7 |
| APPL1 | 57261.765 | 3p21.1-p14.3 | adaptor protein, phosphotyrosine interaction, PH domain and leucine |
| | | | zipper containing 1 |
| IMPDH2 | 49061.762 | 3p21.2 | IMP (inosine 5′-monophosphate) dehydrogenase 2 |
| VPRBP | 51433.298 | 3p21.2 | Vpr (HIV-1) binding protein |
| RAD54L2 | 51575.596 | 3p21.2 | RAD54-like 2 (S. cerevisiae) |
| ACY1 | 52017.3 | 3p21.2 | aminoacylase 1 |
| POC1A | 52109.249 | 3p21.2 | POC1 centriolar protein A |
| ABHD6 | 58223.259 | 3p21.2 | abhydrolase domain containing 6 |
| PXK | 58318.617 | 3p21.2 | PX domain containing serine/threonine kinase |
| KCTD6 | 58477.823 | 3p21.2 | potassium channel tetramerization domain containing 6 |
| ARF4 | 57557.09 | 3p21.2-p21.1 | ADP-ribosylation factor 4 |
| ITGA9 | 37493.813 | 3p21.3 | integrin, alpha 9 |
| CTDSPL | 37903.669 | 3p21.3 | CTD (carboxy-terminal domain, RNA polymerase II, polypeptide A) |
| | | | small phosphatase-like |
| DLEC1 | 38080.696 | 3p21.3 | deleted in lung and esophageal cancer 1 |
| CX3CR1 | 39304.985 | 3p21.3 | chemokine (C-X3-C motif) receptor 1 |
| RPSA | 39449.111 | 3p21.3 | ribosomal protein SA |
| ACKR2 | 42850.964 | 3p21.3 | atypical chemokine receptor 2 |
| CDCP1 | 45123.766 | 3p21.3 | CUB domain containing protein 1 |
| LARS2 | 45430.075 | 3p21.3 | leucyl-tRNA synthetase 2, mitochondrial |
| SACM1L | 45730.754 | 3p21.3 | SAC1 suppressor of actin mutations 1-like (yeast) |
| LZTFL1 | 45864.81 | 3p21.3 | leucine zipper transcription factor-like 1 |
| FYCO1 | 45959.391 | 3p21.3 | FYVE and coiled-coil domain containing 1 |
| CCR3 | 46283.872 | 3p21.3 | chemokine (C-C motif) receptor 3 |
| RTP3 | 46539.485 | 3p21.3 | receptor (chemosensory) transporter protein 3 |
| PTPN23 | 47422.491 | 3p21.3 | protein tyrosine phosphatase, non-receptor type 23 |
| CAMP | 48264.837 | 3p21.3 | cathelicidin antimicrobial peptide |
| USP4 | 49349.218 | 3p21.3 | ubiquitin specific peptidase 4 (proto-oncogene) |
| GPX1 | 49394.609 | 3p21.3 | glutathione peroxidase 1 |
| RHOA | 49396.579 | 3p21.3 | ras homolog family member A |
| RBM6 | 49977.477 | 3p21.3 | RNA binding motif protein 6 |
| RBM5 | 50126.341 | 3p21.3 | RNA binding motif protein 5 |
| SEMA3F | 50192.848 | 3p21.3 | sema domain, immunoglobulin domain (Ig), short basic domain, |
| | | | secreted, (semaphorin) 3F |
| SLC38A3 | 50242.679 | 3p21.3 | solute carrier family 38, member 3 |
| SEMA3B | 50304.99 | 3p21.3 | sema domain, immunoglobulin domain (Ig), short basic domain, |
| | | | secreted, (semaphorin) 3B |
| HYAL3 | 50330.259 | 3p21.3 | hyaluronoglucosaminidase 3 |
| NAT6 | 50333.833 | 3p21.3 | N-acetyltransferase 6 (GCN5-related) |
| HYAL2 | 50355.221 | 3p21.3 | hyaluronoglucosaminidase 2 |
| TUSC2 | 50362.341 | 3p21.3 | tumor suppressor candidate 2 |
| RASSF1 | 50367.217 | 3p21.3 | Ras association (RalGDS/AF-6) domain family member 1 |
| ZMYND10 | 50378.537 | 3p21.3 | zinc finger, MYND-type containing 10 |
| NPRL2 | 50384.919 | 3p21.3 | nitrogen permease regulator-like 2 (S. cerevisiae) |
| CYB561D2 | 50388.126 | 3p21.3 | cytochrome b561 family, member D2 |
| CACNA2D2 | 50400.044 | 3p21.3 | calcium channel, voltage-dependent, alpha 2/delta subunit 2 |
| CISH | 50643.885 | 3p21.3 | cytokine inducible SH2-containing protein |
| MAPKAPK3 | 50654.562 | 3p21.3 | mitogen-activated protein kinase-activated protein kinase 3 |
| TLR9 | 52255.096 | 3p21.3 | toll-like receptor 9 |
| XCR1 | 46062.291 | 3p21.3-p21.1 | chemokine (C motif) receptor 1 |
| PRKAR2A | 48788.093 | 3p21.3-p21.2 | protein kinase, cAMP-dependent, regulatory, type II, alpha |
| LAMB2 | 49158.547 | 3p21.3-p21.2 | laminin, beta 2 (laminin S) |
| HYAL1 | 50337.32 | 3p21.3-p21.2 | hyaluronoglucosaminidase 1 |
| RPL29 | 52027.644 | 3p21.3-p21.2 | ribosomal protein L29 |
| MIR564 | 44903.38 | 3p21.31 | microRNA 564 |
| ZDHHC3 | 44956.753 | 3p21.31 | zinc finger, DHHC-type containing 3 |
| TMEM158 | 45265.956 | 3p21.31 | transmembrane protein 158 (gene/pseudogene) |
| LIMD1 | 45636.323 | 3p21.31 | LIM domains containing 1 |
| CCR9 | 45927.996 | 3p21.31 | chemokine (C-C motif) receptor 9 |
| LTF | 46477.496 | 3p21.31 | lactotransferrin |
| TDGF1 | 46616.045 | 3p21.31 | teratocarcinoma-derived growth factor 1 |
| ALS2CL | 46710.485 | 3p21.31 | ALS2 C-terminal like |
| PRSS50 | 46753.606 | 3p21.31 | protease, serine, 50 |
| SETD2 | 47057.898 | 3p21.31 | SET domain containing 2 |
| SCAP | 47455.184 | 3p21.31 | SREBF chaperone |
| ELP6 | 47537.13 | 3p21.31 | elongator acetyltransferase complex subunit 6 |
| SMARCC1 | 47627.378 | 3p21.31 | SWI/SNF related, matrix associated, actin dependent regulator of |
| | | | chromatin, subfamily c, member 1 |
| MIR1226 | 47891.045 | 3p21.31 | microRNA 1226 |
| NME6 | 48335.589 | 3p21.31 | NME/NM23 nucleoside diphosphate kinase 6 |
| PLXNB1 | 48445.261 | 3p21.31 | plexin B1 |
| TREX1 | 48506.919 | 3p21.31 | three prime repair exonuclease 1 |
| SHISA5 | 48509.197 | 3p21.31 | shisa family member 5 |
| SLC26A6 | 48663.156 | 3p21.31 | solute carrier family 26 (anion exchanger), member 6 |
| CELSR3 | 48673.896 | 3p21.31 | cadherin, EGF LAG seven-pass G-type receptor 3 |
| IP6K2 | 48725.436 | 3p21.31 | inositol hexakisphosphate kinase 2 |
| P4HTM | 49027.341 | 3p21.31 | prolyl 4-hydroxylase, transmembrane (endoplasmic reticulum) |
| MIR425 | 49057.581 | 3p21.31 | microRNA 425 |
| MIR191 | 49058.051 | 3p21.31 | microRNA 191 |
| QRICH1 | 49067.142 | 3p21.31 | glutamine-rich 1 |
| USP19 | 49146.106 | 3p21.31 | ubiquitin specific peptidase 19 |
| KLHDC8B | 49209.018 | 3p21.31 | kelch domain containing 8B |
| NICN1 | 49459.766 | 3p21.31 | nicolin 1 |
| IP6K1 | 49761.728 | 3p21.31 | inositol hexakisphosphate kinase 1 |
| TRAIP | 49866.028 | 3p21.31 | TRAF interacting protein |
| GNAI2 | 50284.326 | 3p21.31 | guanine nucleotide binding protein (G protein), alpha inhibiting activity |
| | | | polypeptide 2 |
| PHF7 | 52446.827 | 3p21.31 | PHD finger protein 7 |
| STAB1 | 52529.356 | 3p21.31 | stabilin 1 |
| SPCS1 | 52739.857 | 3p21.31 | signal peptidase complex subunit 1 homolog (S. cerevisiae) |
| SFMBT1 | 52937.583 | 3p21.31 | Scm-like with four mbt domains 1 |
| PRKCD | 53195.223 | 3p21.31 | protein kinase C, delta |
| ACTR8 | 53901.094 | 3p21.31 | ARP8 actin-related protein 8 homolog (yeast) |
| SELK | 53919.226 | 3p21.31 | selenoprotein K |
| BAP1 | 52435.02 | 3p21.31-p21.2 | BRCA1 associated protein-1 (ubiquitin carboxy-terminal hydrolase) |
| MIR138-1 | 44155.704 | 3p21.32 | microRNA 138-1 |
| EXOSC7 | 45017.741 | 3p21.32 | exosome component 7 |
| WDR48 | 39093.507 | 3p21.33 | WD repeat domain 48 |
| XIRP1 | 39224.706 | 3p21.33 | xin actin-binding repeat containing 1 |
| MOBP | 39509.064 | 3p21.33 | myelin-associated oligodendrocyte basic protein |
| ZNF620 | 40547.53 | 3p21.33 | zinc finger protein 620 |
| KLHL40 | 42727.011 | 3p21.33 | kelch-like family member 40 |
| ABHD5 | 43732.375 | 3p21.33 | abhydrolase domain containing 5 |
| TGFBR2 | 30647.994 | 3p22 | transforming growth factor, beta receptor II (70/80 kDa) |
| MYD88 | 38179.969 | 3p22 | myeloid differentiation primary response 88 |
| ACVR2B | 38495.79 | 3p22 | activin A receptor, type IIB |
| CSRNP1 | 39183.342 | 3p22 | cysteine-serine-rich nuclear protein 1 |
| CCR8 | 39371.197 | 3p22 | chemokine (C-C motif) receptor 8 |
| VIPR1 | 42530.791 | 3p22 | vasoactive intestinal peptide receptor 1 |
| HHATL | 42734.155 | 3p22 | hedgehog acyltransferase-like |
| PFKFB4 | 48555.117 | 3p22-p21 | 6-phosphofructo-2-kinase/fructose-2,6-biphosphatase 4 |
| PTH1R | 46919.236 | 3p22-p21.1 | parathyroid hormone 1 receptor |
| GOLGA4 | 37284.682 | 3p22-p21.3 | golgin A4 |
| PLCD1 | 38048.987 | 3p22-p21.3 | phospholipase C, delta 1 |
| CLEC3B | 45067.759 | 3p22-p21.3 | C-type lectin domain family 3, member B |
| TGM4 | 44916.098 | 3p22-p21.33 | transglutaminase 4 |
| LRRFIP2 | 37094.117 | 3p22.1 | leucine rich repeat (in FLII) interacting protein 2 |
| ULK4 | 41288.09 | 3p22.1 | unc-51 like kinase 4 |
| TRAK1 | 42132.746 | 3p22.1 | trafficking protein, kinesin binding 1 |
| CCK | 42299.318 | 3p22.1 | cholecystokinin |
| HIGD1A | 42824.4 | 3p22.1 | HIG1 hypoxia inducible domain family, member 1A |
| SNRK | 43328.004 | 3p22.1 | SNF related kinase |
| CMTM8 | 32280.171 | 3p22.2 | CKLF-like MARVEL transmembrane domain containing 8 |
| TRANK1 | 36868.308 | 3p22.2 | tetratricopeptide repeat and ankyrin repeat containing 1 |
| MIR26A1 | 38010.895 | 3p22.2 | microRNA 26a-1 |
| OXSR1 | 38207.026 | 3p22.2 | oxidative stress responsive 1 |
| SLC22A13 | 38307.298 | 3p22.2 | solute carrier family 22 (organic anion/urate transporter), member 13 |
| PARP3 | 51976.361 | 3p22.2-p21.1 | poly (ADP-ribose) polymerase family, member 3 |
| CMTM7 | 32433.163 | 3p22.3 | CKLF-like MARVEL transmembrane domain containing 7 |
| TRIM71 | 32859.51 | 3p22.3 | tripartite motif containing 71, E3 ubiquitin protein ligase |
| GLB1 | 33038.1 | 3p22.3 | galactosidase, beta 1 |
| FBXL2 | 33318.937 | 3p22.3 | F-box and leucine-rich repeat protein 2 |
| UBP1 | 33429.829 | 3p22.3 | upstream binding protein 1 (LBP-1a) |
| PDCD6IP | 33840.063 | 3p22.3 | programmed cell death 6 interacting protein |
| MIR128-2 | 35785.968 | 3p22.3 | microRNA 128-2 |
| DCLK3 | 36753.913 | 3p22.3 | doublecortin-like kinase 3 |
| MLH1 | 37034.841 | 3p22.3 | mutL homolog 1 |
| C3orf35 | 37440.968 | 3p22.3 | chromosome 3 open reading frame 35 |
| NGLY1 | 25760.435 | 3p23 | N-glycanase 1 |
| NKTR | 42642.147 | 3p23-p21 | natural killer cell triggering receptor |
| DAZL | 16628.301 | 3p24 | deleted in azoospermia-like |
| KAT2B | 20081.524 | 3p24 | K(lysine) acetyltransferase 2B |
| RARB | 25469.754 | 3p24 | retinoic acid receptor, beta |
| LRRC3B | 26664.3 | 3p24 | leucine rich repeat containing 3B |
| CCR4 | 32993.066 | 3p24-p21.3 | chemokine (C-C motif) receptor 4 |
| RAB5A | 19988.572 | 3p24-p22 | RAB5A, member RAS oncogene family |
| GRIP2 | 14530.619 | 3p24-p23 | glutamate receptor interacting protein 2 |
| RBMS3 | 29322.803 | 3p24-p23 | RNA binding motif, single stranded interacting protein 3 |
| NKIRAS1 | 23933.572 | 3p24.1 | NFKB inhibitor interacting Ras-like 1 |
| RPL15 | 23958.295 | 3p24.1 | ribosomal protein L15 |
| NR1D2 | 23987.612 | 3p24.1 | nuclear receptor subfamily 1, group D, member 2 |
| NEK10 | 27257.097 | 3p24.1 | NIMA-related kinase 10 |
| SLC4A7 | 27414.212 | 3p24.1 | solute carrier family 4, sodium bicarbonate cotransporter, member 7 |
| EOMES | 27757.44 | 3p24.1 | eomesodermin |
| CMC1 | 28283.124 | 3p24.1 | C-x(9)-C motif containing 1 |
| STT3B | 31573.993 | 3p24.1 | STT3B, subunit of the oligosaccharyltransferase complex (catalytic) |
| UBE2E2 | 23244.784 | 3p24.2 | ubiquitin-conjugating enzyme E2E 2 |
| UBE2E1 | 23851.934 | 3p24.2 | ubiquitin-conjugating enzyme E2E 1 |
| THRB | 24158.645 | 3p24.2 | thyroid hormone receptor, beta |
| TOP2B | 25639.396 | 3p24.2 | topoisomerase (DNA) II beta 180 kDa |
| SH3BP5 | 15295.863 | 3p24.3 | SH3-domain binding protein 5 (BTK-associated) |
| RFTN1 | 16357.352 | 3p24.3 | raftlin, lipid raft linker 1 |
| SATB1 | 18389.133 | 3p24.3 | SATB homeobox 1 |
| KCNH8 | 19190.017 | 3p24.3 | potassium voltage-gated channel, subfamily H (eag-related), member 8 |
| SGOL1 | 20209.936 | 3p24.3 | shugoshin-like 1 (S. pombe) |
| RNF123 | 49726.95 | 3p24.3 | ring finger protein 123 |
| UBA3 | 69103.881 | 3p24.3-p13 | ubiquitin-like modifier activating enzyme 3 |
| DHX30 | 47844.399 | 3p24.3-p22.1 | DEAH (Asp-Glu-Ala-His) box helicase 30 |
| ATRIP | 48488.114 | 3p24.3-p22.1 | ATR interacting protein |
| CAV3 | 8775.486 | 3p25 | caveolin 3 |
| OXTR | 8792.095 | 3p25 | oxytocin receptor |
| ARPC4 | 9834.179 | 3p25 | actin related protein 2/3 complex, subunit 4, 20 kDa |
| CIDEC | 9908.394 | 3p25 | cell death-inducing DFFA-like effector c |
| HRH1 | 11294.385 | 3p25 | histamine receptor H1 |
| TIMP4 | 12194.568 | 3p25 | TIMP metallopeptidase inhibitor 4 |
| PPARG | 12329.349 | 3p25 | peroxisome proliferator-activated receptor gamma |
| RAF1 | 12625.1 | 3p25 | Raf-1 proto-oncogene, serine/threonine kinase |
| NUP210 | 13357.73 | 3p25 | nucleoporin 210 kDa |
| WNT7A | 13860.082 | 3p25 | wingless-type MMTV integration site family, member 7A |
| XPC | 14186.648 | 3p25 | xeroderma pigmentosum, complementation group C |
| NR2C2 | 14989.091 | 3p25 | nuclear receptor subfamily 2, group C, member 2 |
| BTD | 15643.252 | 3p25 | biotinidase |
| RAD18 | 8918.88 | 3p25-p24 | RAD18 homolog (S. cerevisiae) |
| FBLN2 | 13590.625 | 3p25-p24 | fibulin 2 |
| HDAC11 | 13521.844 | 3p25.1 | histone deacetylase 11 |
| CHCHD4 | 14153.577 | 3p25.1 | coiled-coil-helix-coiled-coil-helix domain containing 4 |
| SLC6A6 | 14444.076 | 3p25.1 | solute carrier family 6 (neurotransmitter transporter), member 6 |
| FGD5 | 14860.469 | 3p25.1 | FYVE, RhoGEF and PH domain containing 5 |
| ANKRD28 | 15708.744 | 3p25.1 | ankyrin repeat domain 28 |
| DPH3 | 16298.568 | 3p25.1 | diphthamide biosynthesis 3 |
| IRAK2 | 10206.563 | 3p25.2 | interleukin-1 receptor-associated kinase 2 |
| VGLL4 | 11597.541 | 3p25.2 | vestigial-like family member 4 |
| IQSEC1 | 12938.542 | 3p25.2 | IQ motif and Sec7 domain 1 |
| LINC00312 | 8613.891 | 3p25.3 | long intergenic non-protein coding RNA 312 |
| SSUH2 | 8661.086 | 3p25.3 | ssu-2 homolog (C. elegans) |
| SRGAP3 | 9022.276 | 3p25.3 | SLIT-ROBO Rho GTPase activating protein 3 |
| THUMPD3 | 9404.717 | 3p25.3 | THUMP domain containing 3 |
| CAMK1 | 9799.029 | 3p25.3 | calcium/calmodulin-dependent protein kinase I |
| TADA3 | 9821.648 | 3p25.3 | transcriptional adaptor 3 |
| TTLL3 | 9851.644 | 3p25.3 | tubulin tyrosine ligase-like family, member 3 |
| IL17RC | 9958.758 | 3p25.3 | interleukin 17 receptor C |
| FANCD2 | 10068.113 | 3p25.3 | Fanconi anemia, complementation group D2 |
| BRK1 | 10157.333 | 3p25.3 | BRICK1, SCAR/WAVE actin-nucleating complex subunit |
| VHL | 10183.319 | 3p25.3 | von Hippel-Lindau tumor suppressor, E3 ubiquitin protein ligase |
| ATP2B2 | 10365.707 | 3p25.3 | ATPase, Ca++ transporting, plasma membrane 2 |
| MIR885 | 10436.173 | 3p25.3 | microRNA 885 |
| SLC6A1 | 11034.42 | 3p25.3 | solute carrier family 6 (neurotransmitter transporter), member 1 |
| PLCL2 | 16926.452 | 3p25.3-p25.1 | phospholipase C-like 2 |
| ATG7 | 11314.01 | 3p25.3-p25.2 | autophagy related 7 |
| CHL1 | 238.279 | 3p26 | cell adhesion molecule L1-like |
| BHLHE40 | 5021.097 | 3p26 | basic helix-loop-helix family, member e40 |
| MTMR14 | 9691.117 | 3p26 | myotubularin related protein 14 |
| OGG1 | 9791.628 | 3p26 | 8-oxoguanine DNA glycosylase |
| IL5RA | 3108.008 | 3p26-p24 | interleukin 5 receptor, alpha |
| LMCD1 | 8543.493 | 3p26-p24 | LIM and cysteine-rich domains 1 |
| CNTN6 | 1134.342 | 3p26-p25 | contactin 6 |
| BRPF1 | 9773.434 | 3p26-p25 | bromodomain and PHD finger containing, 1 |
| GHRL | 10327.434 | 3p26-p25 | ghrelin/obestatin prepropeptide |
| ITPR1 | 4535.032 | 3p26.1 | inositol 1,4,5-trisphosphate receptor, type 1 |
| EDEM1 | 5229.359 | 3p26.1 | ER degradation enhancer, mannosidase alpha-like 1 |
| SETMAR | 4344.988 | 3p26.2 | SET domain and mariner transposase fusion gene |
| CNTN4 | 2280.513 | 3p26.3 | contactin 4 |
| CBLB | 105377.109 | 3q | Cbl proto-oncogene B, E3 ubiquitin protein ligase |
| CLDN18 | 137717.658 | 3q | claudin 18 |
| PROS1 | 93591.881 | 3q11.1 | protein S (alpha) |
| ARL6 | 97483.365 | 3q11.2 | ADP-ribosylation factor-like 6 |
| CPOX | 98298.29 | 3q12 | coproporphyrinogen oxidase |
| SENP7 | 101043.033 | 3q12 | SUMO1/sentrin specific peptidase 7 |
| NR1I2 | 119499.331 | 3q12-q13.3 | nuclear receptor subfamily 1, group I, member 2 |
| EPHA6 | 96533.425 | 3q12.1 | EPH receptor A6 |
| CLDND1 | 98234.317 | 3q12.1 | claudin domain containing 1 |
| FILIP1L | 99566.767 | 3q12.1 | filamin A interacting protein 1 -like |
| ST3GAL6 | 98451.08 | 3q12.2 | ST3 beta-galactoside alpha-2,3-sialyltransferase 6 |
| DCBLD2 | 98514.814 | 3q12.2 | discoidin, CUB and LCCL domain containing 2 |
| TBC1D23 | 99979.661 | 3q12.2 | TBC1 domain family, member 23 |
| TOMM70A | 100082.303 | 3q12.2 | translocase of outer mitochondrial membrane 70 homolog A ((S. |
| | | | cerevisiae |
| LNP1 | 100120.037 | 3q12.2 | leukemia NUP98 fusion partner 1 |
| TMEM45A | 100211.463 | 3q12.2 | transmembrane protein 45A |
| TFG | 100428.134 | 3q12.2 | TRK-fused gene |
| ABI3BP | 100468.179 | 3q12.2 | ABI family, member 3 (NESH) binding protein |
| IMPG2 | 100941.39 | 3q12.2-q12.3 | interphotoreceptor matrix proteoglycan 2 |
| NIT2 | 100053.562 | 3q12.3 | nitrilase family, member 2 |
| GPR128 | 100328.433 | 3q12.3 | G protein-coupled receptor 128 |
| TRAT1 | 108541.631 | 3q13 | T cell receptor associated transmembrane adaptor 1 |
| CD200R1 | 112641.532 | 3q13 | CD200 receptor 1 |
| HCLS1 | 121350.245 | 3q13 | hematopoietic cell-specific Lyn substrate 1 |
| GOLGB1 | 121382.046 | 3q13 | golgin B1 |
| UMPS | 124449.213 | 3q13 | uridine monophosphate synthetase |
| MAGEF1 | 184428.155 | 3q13 | melanoma antigen family F, 1 |
| PARP9 | 122246.76 | 3q13-q21 | poly (ADP-ribose) polymerase family, member 9 |
| ALCAM | 105085.557 | 3q13.1 | activated leukocyte cell adhesion molecule |
| RETNLB | 108474.486 | 3q13.1 | resistin like beta |
| GUCA1C | 108626.642 | 3q13.1 | guanylate cyclase activator 1C |
| CD47 | 107761.941 | 3q13.1-q13.2 | CD47 molecule |
| MIR548A3 | 103903.476 | 3q13.11 | microRNA 548a-3 |
| CCDC54 | 107096.188 | 3q13.12 | coiled-coil domain containing 54 |
| KIAA1524 | 108268.718 | 3q13.13 | KIAA1524 |
| DPPA2 | 109012.635 | 3q13.13 | developmental pluripotency associated 2 |
| DPPA4 | 109044.988 | 3q13.13 | developmental pluripotency associated 4 |
| PHLDB2 | 111451.327 | 3q13.13 | pleckstrin homology-like domain, family B, member 2 |
| GCSAM | 111839.688 | 3q13.13 | germinal center-associated, signaling and motility |
| CD96 | 111260.926 | 3q13.13-q13.2 | CD96 molecule |
| PLCXD2 | 111393.523 | 3q13.2 | phosphatidylinositol-specific phospholipase C, X domain containing 2 |
| CD200 | 112051.916 | 3q13.2 | CD200 molecule |
| BTLA | 112182.813 | 3q13.2 | B and T lymphocyte associated |
| CCDC80 | 112323.233 | 3q13.2 | coiled-coil domain containing 80 |
| BOC | 112931.375 | 3q13.2 | BOC cell adhesion associated, oncogene regulated |
| ZBTB20 | 114033.347 | 3q13.2 | zinc finger and BTB domain containing 20 |
| LSAMP | 115521.21 | 3q13.2-q21 | limbic system-associated membrane protein |
| DRD3 | 113847.499 | 3q13.3 | dopamine receptor D3 |
| GSK3B | 119540.802 | 3q13.3 | glycogen synthase kinase 3 beta |
| POLQ | 121150.273 | 3q13.3 | polymerase (DNA directed), theta |
| CD80 | 119243.14 | 3q13.3-q21 | CD80 molecule |
| TRH | 129693.236 | 3q13.3-q21 | thyrotropin-releasing hormone |
| NAA50 | 113435.307 | 3q13.31 | N(alpha)-acetyltransferase 50, NatE catalytic subunit |
| TIGIT | 114012.833 | 3q13.31 | T cell immunoreceptor with Ig and ITIM domains |
| TUSC7 | 116428.635 | 3q13.31 | tumor suppressor candidate 7 (non-protein coding) |
| ADPRH | 119298.28 | 3q13.31-q13.33 | ADP-ribosylarginine hydrolase |
| UPK1B | 118892.425 | 3q13.32 | uroplakin 1B |
| POGLUT1 | 119187.785 | 3q13.33 | protein O-glucosyltransferase 1 |
| POPDC2 | 119360.908 | 3q13.33 | popeye domain containing 2 |
| COX17 | 119388.372 | 3q13.33 | COX17 cytochrome c oxidase copper chaperone |
| FSTL1 | 120113.061 | 3q13.33 | follistatin-like 1 |
| MIR198 | 120114.515 | 3q13.33 | microRNA 198 |
| RABL3 | 120405.528 | 3q13.33 | RAB, member of RAS oncogene family-like 3 |
| STXBP5L | 120627.05 | 3q13.33-q21.1 | syntaxin binding protein 5-like |
| CD86 | 121774.209 | 3q21 | CD86 molecule |
| CSTA | 122044.011 | 3q21 | cystatin A (stefin A) |
| KPNA1 | 122140.748 | 3q21 | karyopherin alpha 1 (importin alpha 5) |
| PARP14 | 122399.672 | 3q21 | poly (ADP-ribose) polymerase family, member 14 |
| MYLK | 123331.143 | 3q21 | myosin light chain kinase |
| OSBPL11 | 125247.702 | 3q21 | oxysterol binding protein-like 11 |
| MCM2 | 127317.2 | 3q21 | minichromosome maintenance complex component 2 |
| ABTB1 | 127391.781 | 3q21 | ankyrin repeat and BTB (POZ) domain containing 1 |
| RUVBL1 | 127799.8 | 3q21 | RuvB-like AAA ATPase 1 |
| GATA2 | 128198.265 | 3q21 | GATA binding protein 2 |
| C3orf27 | 128290.843 | 3q21 | chromosome 3 open reading frame 27 |
| RAB7A | 128444.979 | 3q21 | RAB7A, member RAS oncogene family |
| CNBP | 128886.658 | 3q21 | CCHC-type zinc finger, nucleic acid binding protein |
| IFT122 | 129158.879 | 3q21 | intraflagellar transport 122 |
| TF | 133464.977 | 3q21 | transferrin |
| SLCO2A1 | 133651.54 | 3q21 | solute carrier organic anion transporter family, member 2A1 |
| NCK1 | 136649.317 | 3q21 | NCK adaptor protein 1 |
| AMOTL2 | 134074.187 | 3q21-q22 | angiomotin like 2 |
| EPHB1 | 134514.099 | 3q21-q23 | EPH receptor B1 |
| RBP1 | 139245.247 | 3q21-q23 | retinol binding protein 1, cellular |
| GTF2E1 | 120461.558 | 3q21-q24 | general transcription factor IIE, polypeptide 1, alpha 56 kDa |
| RHO | 129247.482 | 3q21-q24 | rhodopsin |
| TM4SF1 | 149086.805 | 3q21-q25 | transmembrane 4 L six family member 1 |
| GAP43 | 115342.151 | 3q21-qter | growth associated protein 43 |
| PIK3CB | 138371.54 | 3q21-qter | phosphatidylinositol-4,5-bisphosphate 3-kinase, catalytic subunit beta |
| EPHB3 | 184279.587 | 3q21-qter | EPH receptor B3 |
| KNG1 | 186435.098 | 3q21-qter | kininogen 1 |
| FBXO40 | 121312.17 | 3q21.1 | F-box protein 40 |
| EAF2 | 121554.034 | 3q21.1 | ELL associated factor 2 |
| ILDR1 | 121706.17 | 3q21.1 | immunoglobulin-like domain containing receptor 1 |
| CASR | 121902.53 | 3q21.1 | calcium-sensing receptor |
| FAM162A | 122103.023 | 3q21.1 | family with sequence similarity 162, member A |
| DTX3L | 122283.185 | 3q21.1 | deltex 3 like, E3 ubiquitin ligase |
| HSPBAP1 | 122458.844 | 3q21.1 | HSPB (heat shock 27 kDa) associated protein 1 |
| DIRC2 | 122513.901 | 3q21.1 | disrupted in renal carcinoma 2 |
| PDIA5 | 122785.856 | 3q21.1 | protein disulfide isomerase family A, member 5 |
| ROPN1 | 123687.879 | 3q21.1 | rhophilin associated tail protein 1 |
| IGSF11 | 118619.479 | 3q21.2 | immunoglobulin superfamily, member 11 |
| ITGB5 | 124481.795 | 3q21.2 | integrin, beta 5 |
| MUC13 | 124624.289 | 3q21.2 | mucin 13, cell surface associated |
| SLC12A8 | 124801.48 | 3q21.2 | solute carrier family 12, member 8 |
| ZNF148 | 124944.513 | 3q21.2 | zinc finger protein 148 |
| SNX4 | 125165.488 | 3q21.2 | sorting nexin 4 |
| ALDH1L1 | 125822.404 | 3q21.2 | aldehyde dehydrogenase 1 family, member L1 |
| CCDC37 | 126113.782 | 3q21.2 | coiled-coil domain containing 37 |
| PLXNA1 | 126707.437 | 3q21.2 | plexin A1 |
| CHCHD6 | 126423.063 | 3q21.3 | coiled-coil-helix-coiled-coil-helix domain containing 6 |
| SEC61A1 | 127771.212 | 3q21.3 | Sec61 alpha 1 subunit (S. cerevisiae) |
| EEFSEC | 127872.313 | 3q21.3 | eukaryotic elongation factor, selenocysteine-tRNA-specific |
| DNAJB8 | 128181.275 | 3q21.3 | DnaJ (Hsp40) homolog, subfamily B, member 8 |
| RPN1 | 128338.813 | 3q21.3 | ribophorin I |
| RAB43 | 128806.412 | 3q21.3 | RAB43, member RAS oncogene family |
| H1FX | 129033.614 | 3q21.3 | H1 histone family, member X |
| MBD4 | 129149.787 | 3q21.3 | methyl-CpG binding domain protein 4 |
| PLXND1 | 129274.056 | 3q21.3 | plexin D1 |
| TMCC1 | 129366.635 | 3q21.3 | transmembrane and coiled-coil domain family 1 |
| ASTE1 | 130732.717 | 3q21.3 | asteroid homolog 1 (Drosophila) |
| NUDT16 | 131100.515 | 3q21.3 | nudix (nucleoside diphosphate linked moiety X)-type motif 16 |
| ACKR4 | 132316.081 | 3q22 | atypical chemokine receptor 4 |
| UBA5 | 132379.14 | 3q22 | ubiquitin-like modifier activating enzyme 5 |
| ARMC8 | 137906.09 | 3q22 | armadillo repeat containing 8 |
| RASA2 | 141205.926 | 3q22-q23 | RAS p21 protein activator 2 |
| RNF7 | 141457.051 | 3q22-q24 | ring finger protein 7 |
| MINA | 97660.661 | 3q22.1 | MYC induced nuclear antigen |
| PIK3R4 | 130397.778 | 3q22.1 | phosphoinositide-3-kinase, regulatory subunit 4 |
| NEK11 | 130745.694 | 3q22.1 | NIMA-related kinase 11 |
| CPNE4 | 131252.404 | 3q22.1 | copine IV |
| ACPP | 132036.211 | 3q22.1 | acid phosphatase, prostate |
| DNAJC13 | 132136.553 | 3q22.1 | DnaJ (Hsp40) homolog, subfamily C, member 13 |
| ACAD11 | 132276.982 | 3q22.1 | acyl-CoA dehydrogenase family, member 11 |
| BFSP2 | 133118.839 | 3q22.1 | beaded filament structural protein 2, phakinin |
| TOPBP1 | 133319.449 | 3q22.1 | topoisomerase (DNA) II binding protein 1 |
| SRPRB | 133502.877 | 3q22.1 | signal recognition particle receptor, B subunit |
| RAB6B | 133543.08 | 3q22.1 | RAB6B, member RAS oncogene family |
| C3orf36 | 133646.99 | 3q22.1 | chromosome 3 open reading frame 36 |
| RYK | 133875.978 | 3q22.1 | receptor-like tyrosine kinase |
| ANAPC13 | 134196.546 | 3q22.1 | anaphase promoting complex subunit 13 |
| CEP63 | 134204.575 | 3q22.1 | centrosomal protein 63 kDa |
| KY | 134318.765 | 3q22.1 | kyphoscoliosis peptidase |
| PPP2R3A | 135684.515 | 3q22.2-q22.3 | protein phosphatase 2, regulatory subunit B″, alpha |
| STAG1 | 136055.999 | 3q22.2-q22.3 | stromal antigen 1 |
| SLC35G2 | 136537.861 | 3q22.3 | solute carrier family 35, member G2 |
| IL20RB | 136676.707 | 3q22.3 | interleukin 20 receptor beta |
| NME9 | 137980.279 | 3q22.3 | NME/NM23 family member 9 |
| MRAS | 138067.508 | 3q22.3 | muscle RAS oncogene homolog |
| FAIM | 138327.542 | 3q23 | Fas apoptotic inhibitory molecule |
| FOXL2 | 138663.066 | 3q23 | forkhead box L2 |
| RBP2 | 139171.726 | 3q23 | retinol binding protein 2, cellular |
| SLC25A36 | 140660.662 | 3q23 | solute carrier family 25 (pyrimidine nucleotide carrier), member 36 |
| SPSB4 | 140770.743 | 3q23 | splA/ryanodine receptor domain and SOCS box containing 4 |
| TFDP2 | 141663.27 | 3q23 | transcription factor Dp-2 (E2F dimerization partner 2) |
| XRN1 | 142025.449 | 3q23 | 5′-3′exoribonuclease 1 |
| ATR | 142168.077 | 3q23 | ATR serine/threonine kinase |
| PLS1 | 142315.229 | 3q23 | plastin 1 |
| TRPC1 | 142443.266 | 3q23 | transient receptor potential cation channel, subfamily C, member 1 |
| PLSCR1 | 146232.967 | 3q23 | phospholipid scramblase 1 |
| WWTR1 | 149235.022 | 3q23-q24 | WW domain containing transcription regulator 1 |
| CP | 148890.29 | 3q23-q25 | ceruloplasmin (ferroxidase) |
| CHST2 | 142838.618 | 3q24 | carbohydrate (N-acetylglucosamine-6-O) sulfotransferase 2 |
| PLOD2 | 145787.228 | 3q24 | procollagen-lysine, 2-oxoglutarate 5-dioxygenase 2 |
| ZIC4 | 147103.835 | 3q24 | Zic family member 4 |
| ZIC1 | 147127.181 | 3q24 | Zic family member 1 |
| AGTR1 | 148415.658 | 3q24 | angiotensin II receptor, type 1 |
| CPB1 | 148545.588 | 3q24 | carboxypeptidase B1 (tissue) |
| GPR87 | 151011.876 | 3q24 | G protein-coupled receptor 87 |
| P2RY12 | 151054.631 | 3q24-q25 | purinergic receptor P2Y, G-protein coupled, 12 |
| TM4SF4 | 149192.368 | 3q25 | transmembrane 4 L six family member 4 |
| SIAH2 | 150458.91 | 3q25 | siah E3 ubiquitin protein ligase 2 |
| MBNL1 | 151985.829 | 3q25 | muscleblind-like splicing regulator 1 |
| PLCH1 | 155197.671 | 3q25 | phospholipase C, eta 1 |
| PTX3 | 157154.58 | 3q25 | pentraxin 3, long |
| MLF1 | 158288.953 | 3q25 | myeloid leukemia factor 1 |
| RNF13 | 149530.475 | 3q25.1 | ring finger protein 13 |
| PFN2 | 149682.691 | 3q25.1 | profilin 2 |
| SERP1 | 150259.78 | 3q25.1 | stress-associated endoplasmic reticulum protein 1 |
| EIF2A | 150264.574 | 3q25.1 | eukaryotic translation initiation factor 2A, 65 kDa |
| AADACL2 | 151451.704 | 3q25.1 | arylacetamide deacetylase-like 2 |
| AADAC | 151531.861 | 3q25.1 | arylacetamide deacetylase |
| HLTF | 148747.904 | 3q25.1-q26.1 | helicase-like transcription factor |
| P2RY1 | 152552.736 | 3q25.2 | purinergic receptor P2Y, G-protein coupled, 1 |
| RAP2B | 152880.001 | 3q25.2 | RAP2B, member of RAS oncogene family |
| C3orf79 | 153202.284 | 3q25.2 | chromosome 3 open reading frame 79 |
| ARHGEF26 | 153838.792 | 3q25.2 | Rho guanine nucleotide exchange factor (GEF) 26 |
| DHX36 | 153993.457 | 3q25.2 | DEAH (Asp-Glu-Ala-His) box polypeptide 36 |
| MME | 154797.436 | 3q25.2 | membrane metallo-endopeptidase |
| SI | 164696.686 | 3q25.2-q26.2 | sucrase-isomaltase (alpha-glucosidase) |
| GMPS | 155588.325 | 3q25.31 | guanine monphosphate synthase |
| SSR3 | 156257.929 | 3q25.31 | signal sequence receptor, gamma (translocon-associated protein |
| | | | gamma) |
| TIPARP | 156392.715 | 3q25.31 | TCDD-inducible poly(ADP-ribose) polymerase |
| CCNL1 | 156865.586 | 3q25.31 | cyclin L1 |
| C3orf55 | 157261.133 | 3q25.32 | chromosome 3 open reading frame 55 |
| SHOX2 | 157813.8 | 3q25.32 | short stature homeobox 2 |
| LXN | 158384.203 | 3q25.32 | latexin |
| RARRES1 | 158422.44 | 3q25.32 | retinoic acid receptor responder (tazarotene induced) 1 |
| IQCJ | 158787.041 | 3q25.32 | IQ motif containing J |
| IL12A | 159706.623 | 3q25.33 | interleukin 12A |
| MIR15B | 160122.376 | 3q25.33 | microRNA 15b |
| MIR16-2 | 160122.533 | 3q25.33 | microRNA 16-2 |
| KPNA4 | 160212.783 | 3q25.33 | karyopherin alpha 4 (importin alpha 3) |
| TRIM59 | 160153.291 | 3q26 | tripartite motif containing 59 |
| SKIL | 170077.411 | 3q26 | SKI-like proto-oncogene |
| PLD1 | 171318.195 | 3q26 | phospholipase D1, phosphatidylcholine-specific |
| TNFSF10 | 172223.298 | 3q26 | tumor necrosis factor (ligand) superfamily, member 10 |
| CLCN2 | 184063.973 | 3q26-qter | chloride channel, voltage-sensitive 2 |
| SMC4 | 160117.092 | 3q26.1 | structural maintenance of chromosomes 4 |
| PPM1L | 160473.996 | 3q26.1 | protein phosphatase, Mg2+/Mn2+ dependent, 1L |
| OTOL1 | 161214.596 | 3q26.1 | otolin 1 |
| SERPINI2 | 167159.577 | 3q26.1 | serpin peptidase inhibitor, clade I (pancpin), member 2 |
| PDCD10 | 167401.695 | 3q26.1 | programmed cell death 10 |
| BCHE | 165490.692 | 3q26.1-q26.2 | butyrylcholinesterase |
| ECT2 | 172468.475 | 3q26.1-q26.2 | epithelial cell transforming 2 |
| MECOM | 168801.287 | 3q26.2 | MDS1 and EVI1 complex locus |
| TERC | 169482.398 | 3q26.2 | telomerase RNA component |
| SEC62 | 169684.58 | 3q26.2 | SEC62 homolog (S. cerevisiae) |
| EIF5A2 | 170606.204 | 3q26.2 | eukaryotic translation initiation factor 5A2 |
| CLDN11 | 170139.028 | 3q26.2-q26.3 | claudin 11 |
| USP13 | 179370.933 | 3q26.2-q26.3 | ubiquitin specific peptidase 13 (isopeptidase T-3) |
| GPR160 | 169755.735 | 3q26.2-q27 | G protein-coupled receptor 160 |
| SLC2A2 | 170714.137 | 3q26.2-q27 | solute carrier family 2 (facilitated glucose transporter), member 2 |
| TRA2B | 185632.358 | 3q26.2-q27 | transformer 2 beta homolog (Drosophila) |
| APOD | 195295.573 | 3q26.2-qter | apolipoprotein D |
| TFRC | 195776.155 | 3q26.2-qter | transferrin receptor |
| PRKCI | 169940.22 | 3q26.3 | protein kinase C, iota |
| PIK3CA | 178866.311 | 3q26.3 | phosphatidylinositol-4,5-bisphosphate 3-kinase, catalytic subunit alpha |
| DCUN1D1 | 182660.559 | 3q26.3 | DCN1, defective in cullin neddylation 1, domain containing 1 |
| KCNMB3 | 178957.537 | 3q26.3-q27 | potassium large conductance calcium-activated channel, subfamily M |
| | | | beta member 3 |
| SOX2 | 181429.712 | 3q26.3-q27 | SRY (sex determining region Y)-box 2 |
| LAMP3 | 182840.003 | 3q26.3-q27 | lysosomal-associated membrane protein 3 |
| EHHADH | 184908.412 | 3q26.3-q28 | enoyl-CoA, hydratase/3-hydroxyacyl CoA dehydrogenase |
| TNIK | 170780.292 | 3q26.31 | TRAF2 and NCK interacting kinase |
| FNDC3B | 171758.344 | 3q26.31 | fibronectin type III domain containing 3B |
| GHSR | 172165.333 | 3q26.31 | growth hormone secretagogue receptor |
| NCEH1 | 172348.435 | 3q26.31 | neutral cholesterol ester hydrolase 1 |
| SPATA16 | 172607.147 | 3q26.31 | spermatogenesis associated 16 |
| PHC3 | 169805.368 | 3q26.32 | polyhomeotic homolog 3 (Drosophila) |
| NLGN1 | 173116.238 | 3q26.32 | neuroligin 1 |
| KCNMB2 | 178276.488 | 3q26.32 | potassium large conductance calcium-activated channel, subfamily M, |
| | | | beta member 2 |
| ZMAT3 | 178735.011 | 3q26.32 | zinc finger, matrin-type 3 |
| MFN1 | 179065.48 | 3q26.32 | mitofusin 1 |
| TBL1XR1 | 176738.542 | 3q26.33 | transducin (beta)-like 1 X-linked receptor 1 |
| ACTL6A | 179280.708 | 3q26.33 | actin-like 6A |
| CCDC39 | 180331.796 | 3q26.33 | coiled-coil domain containing 39 |
| DNAJC19 | 180701.498 | 3q26.33 | DnaJ (Hsp40) homolog, subfamily C, member 19 |
| SOX2-OT | 181328.122 | 3q26.33 | SOX2 overlapping transcript |
| ATP11B | 182511.291 | 3q27 | ATPase, class VI, type 11B |
| MCF2L2 | 182895.792 | 3q27 | MCF.2 cell line derived transforming sequence-like 2 |
| ABCC5 | 183701.541 | 3q27 | ATP-binding cassette, sub-family C (CFTR/MRP), member 5 |
| DVL3 | 183873.284 | 3q27 | dishevelled segment polarity protein 3 |
| THPO | 184089.723 | 3q27 | thrombopoietin |
| CHRD | 184097.861 | 3q27 | chordin |
| C3orf70 | 184795.838 | 3q27 | chromosome 3 open reading frame 70 |
| MAP3K13 | 185000.729 | 3q27 | mitogen-activated protein kinase kinase kinase 13 |
| LIPH | 185225.57 | 3q27 | lipase, member H |
| DNAJB11 | 186288.465 | 3q27 | DnaJ (Hsp40) homolog, subfamily B, member 11 |
| HRG | 186383.747 | 3q27 | histidine-rich glycoprotein |
| RFC4 | 186507.682 | 3q27 | replication factor C (activator 1) 4, 37 kDa |
| ADIPOQ | 186560.463 | 3q27 | adiponectin, C1Q and collagen domain containing |
| BCL6 | 187439.165 | 3q27 | B-cell CLL/lymphoma 6 |
| DGKG | 185864.99 | 3q27-q28 | diacylglycerol kinase, gamma 90 kDa |
| ST6GAL1 | 186739.665 | 3q27-q28 | ST6 beta-galactosamide alpha-2,6-sialyltranferase 1 |
| MASP1 | 186964.142 | 3q27-q28 | mannan-binding lectin serine peptidase 1 (C4/C2 activating component |
| | | | of Ra-reactive factor) |
| LPP | 187943.193 | 3q27-q28 | LIM domain containing preferred translocation partner in lipoma |
| TP63 | 189349.216 | 3q27-q29 | tumor protein p63 |
| EIF4G1 | 184032.283 | 3q27-qter | eukaryotic translation initiation factor 4 gamma, 1 |
| ZNF639 | 179041.551 | 3q27.1 | zinc finger protein 639 |
| GNB4 | 179113.876 | 3q27.1 | guanine nucleotide binding protein (G protein), beta polypeptide 4 |
| ABCF3 | 183903.863 | 3q27.1 | ATP-binding cassette, sub-family F (GCN20), member 3 |
| CAMK2N2 | 183977.003 | 3q27.1 | calcium/calmodulin-dependent protein kinase II inhibitor 2 |
| ECE2 | 183993.799 | 3q27.1 | endothelin converting enzyme 2 |
| VPS8 | 184529.931 | 3q27.2 | vacuolar protein sorting 8 homolog (S. cerevisiae) |
| IGF2BP2 | 185361.527 | 3q27.2 | insulin-like growth factor 2 mRNA binding protein 2 |
| PSMD2 | 184018.369 | 3q27.3 | proteasome (prosome, macropain) 26S subunit, non-ATPase, 2 |
| AHSG | 186330.85 | 3q27.3 | alpha-2-HS-glycoprotein |
| SNORA63 | 186505.088 | 3q27.3 | small nucleolar RNA, H/ACA box 63 |
| RTP1 | 186915.274 | 3q27.3 | receptor (chemosensory) transporter protein 1 |
| FXR1 | 180630.234 | 3q28 | fragile X mental retardation, autosomal homolog 1 |
| B3GNT5 | 182971.032 | 3q28 | UDP-GlcNAc:betaGal beta-1,3-N-acetylglucosaminyltransferase 5 |
| AP2M1 | 183892.634 | 3q28 | adaptor-related protein complex 2, mu 1 subunit |
| POLR2H | 184080.771 | 3q28 | polymerase (RNA) II (DNA directed) polypeptide H |
| SENP2 | 185304.031 | 3q28 | SUMO1/sentrin/SMT3 specific peptidase 2 |
| ETV5 | 185764.106 | 3q28 | ets variant 5 |
| EIF4A2 | 186501.361 | 3q28 | eukaryotic translation initiation factor 4A2 |
| SST | 187386.694 | 3q28 | somatostatin |
| TPRG1 | 188889.763 | 3q28 | tumor protein p63 regulated 1 |
| CLDN16 | 190105.661 | 3q28 | claudin 16 |
| TMEM207 | 190146.444 | 3q28 | transmembrane protein 207 |
| IL1RAP | 190231.84 | 3q28 | interleukin 1 receptor accessory protein |
| CCDC50 | 191046.874 | 3q28 | coiled-coil domain containing 50 |
| PYDC2 | 191178.952 | 3q28 | pyrin domain containing 2 |
| FGF12 | 191857.182 | 3q28 | fibroblast growth factor 12 |
| CLDN1 | 190023.49 | 3q28-q29 | claudin 1 |
| OPA1 | 193310.933 | 3q28-q29 | optic atrophy 1 (autosomal dominant) |
| HES1 | 193853.931 | 3q28-q29 | hes family bHLH transcription factor 1 |
| MFI2 | 196728.612 | 3q28-q29 | antigen p97 (melanoma associated) identified by monoclonal |
| | | | antibodies 133.2 and 96.5 |
| PPP4R2 | 73046.119 | 3q29 | protein phosphatase 4, regulatory subunit 2 |
| LEPREL1 | 189674.517 | 3q29 | leprecan-like 1 |
| HRASLS | 192958.917 | 3q29 | HRAS-like suppressor |
| LRRC15 | 194075.976 | 3q29 | leucine rich repeat containing 15 |
| PPP1R2 | 195241.218 | 3q29 | protein phosphatase 1, regulatory (inhibitor) subunit 2 |
| MUC20 | 195447.753 | 3q29 | mucin 20, cell surface associated |
| MUC4 | 195473.638 | 3q29 | mucin 4, cell surface associated |
| TNK2 | 195590.236 | 3q29 | tyrosine kinase, non-receptor, 2 |
| PCYT1A | 195965.253 | 3q29 | phosphate cytidylyltransferase 1, choline, alpha |
| RNF168 | 196195.657 | 3q29 | ring finger protein 168, E3 ubiquitin protein ligase |
| WDR53 | 196281.059 | 3q29 | WD repeat domain 53 |
| FBXO45 | 196295.725 | 3q29 | F-box protein 45 |
| PAK2 | 196466.728 | 3q29 | p21 protein (Cdc42/Rac)-activated kinase 2 |
| SENP5 | 196594.727 | 3q29 | SUMO1/sentrin specific peptidase 5 |
| DLG1 | 196769.431 | 3q29 | discs, large homolog 1 (Drosophila) |
| BDH1 | 197236.654 | 3q29 | 3-hydroxybutyrate dehydrogenase, type 1 |
| FYTTD1 | 197476.424 | 3q29 | forty-two-three domain containing 1 |
| LRCH3 | 197518.145 | 3q29 | leucine-rich repeats and calponin homology (CH) domain containing 3 |
| IQCG | 197615.946 | 3q29 | IQ motif containing G |

*Information obtained from http://atlasgeneticsoncology.org/Indexbychrom/idxg_3.html (last accessed Sep. 11, 2014)

TABLE 7
List of cancer genes on human chromosome 5*

| | GoldenPath | | |
|---|---|---|---|
| Symbol | (Mb) | Location | Description |
| C7 | 40909.599 | 5 | complement component 7 |
| CKMT2 | 80529.139 | 5 | creatine kinase, mitochondrial 2 (sarcomeric) |
| CAMK2A | 149599.054 | 5 | calcium/calmodulin-dependent protein kinase II alpha |
| FBXO4 | 41925.356 | 5p12 | F-box protein 4 |
| ANXA2R | 43039.182 | 5p12 | annexin A2 receptor |
| ZNF131 | 43121.642 | 5p12 | zinc finger protein 131 |
| CCL28 | 43381.6 | 5p12 | chemokine (C-C motif) ligand 28 |
| NNT | 43602.791 | 5p12 | nicotinamide nucleotide transhydrogenase |
| HCN1 | 45255.052 | 5p12 | hyperpolarization activated cyclic nucleotide-gated potassium channel 1 |
| C1QTNF3 | 34017.963 | 5p13 | C1q and tumor necrosis factor related protein 3 |
| RAD1 | 34905.366 | 5p13 | RAD1 homolog (S. pombe) |
| IL7R | 35856.977 | 5p13 | interleukin 7 receptor |
| SKP2 | 36152.145 | 5p13 | S-phase kinase-associated protein 2, E3 ubiquitin protein ligase |
| SLC1A3 | 36606.457 | 5p13 | solute carrier family 1 (glial high affinity glutamate transporter), |
| | | | member 3 |
| DAB2 | 39371.776 | 5p13 | Dab, mitogen-responsive phosphoprotein, homolog 2 (Drosophila) |
| OXCT1 | 41730.167 | 5p13 | 3-oxoacid CoA transferase 1 |
| LIFR | 38475.065 | 5p13-p12 | leukemia inhibitory factor receptor alpha |
| FGF10 | 44305.097 | 5p13-p12 | fibroblast growth factor 10 |
| RICTOR | 38938.023 | 5p13.1 | RPTOR independent companion of MTOR, complex 2 |
| FYB | 39105.354 | 5p13.1 | FYN binding protein |
| PTGER4 | 40680.032 | 5p13.1 | prostaglandin E receptor 4 (subtype EP4) |
| PRKAA1 | 40759.481 | 5p13.1 | protein kinase, AMP-activated, alpha 1 catalytic subunit |
| CARD6 | 40841.41 | 5p13.1 | caspase recruitment domain family, member 6 |
| PLCXD3 | 41307.048 | 5p13.1 | phosphatidylinositol-specific phospholipase C, X domain containing 3 |
| GDNF | 37812.779 | 5p13.1-p12 | glial cell derived neurotrophic factor |
| GOLPH3 | 32124.824 | 5p13.2 | golgi phosphoprotein 3 (coat-protein) |
| AMACR | 33987.091 | 5p13.2 | alpha-methylacyl-CoA racemase |
| NADK2 | 36192.691 | 5p13.2 | NAD kinase 2, mitochondrial |
| NIPBL | 36876.861 | 5p13.2 | Nipped-B homolog (Drosophila) |
| WDR70 | 37379.412 | 5p13.2 | WD repeat domain 70 |
| OSMR | 38845.96 | 5p13.2 | oncostatin M receptor |
| CDH6 | 31193.762 | 5p13.3 | cadherin 6, type 2, K-cadherin (fetal kidney) |
| SUB1 | 32585.605 | 5p13.3 | SUB1 homolog (S. cerevisiae) |
| SLC45A2 | 33945.972 | 5p13.3 | solute carrier family 45, member 2 |
| C9 | 39284.378 | 5p14-p12 | complement component 9 |
| GHR | 42423.877 | 5p14-p12 | growth hormone receptor |
| PRLR | 35055.802 | 5p14-p13 | prolactin receptor |
| TRIO | 14143.829 | 5p14-p15.1 | trio Rho guanine nucleotide exchange factor |
| PDZD2 | 31799.031 | 5p14.1 | PDZ domain containing 2 |
| PRDM9 | 23507.724 | 5p14.2 | PR domain containing 9 |
| CDH18 | 19473.155 | 5p14.3 | cadherin 18, type 2 |
| CDH12 | 21750.973 | 5p14.3 | cadherin 12, type 2 (N-cadherin 2) |
| ERBB2IP | 65222.382 | 5p14.3-q12.3 | erbb2 interacting protein |
| SDHA | 218.356 | 5p15 | succinate dehydrogenase complex, subunit A, flavoprotein (Fp) |
| TRIP13 | 892.969 | 5p15 | thyroid hormone receptor interactor 13 |
| SLC12A7 | 1050.489 | 5p15 | solute carrier family 12 (potassium/chloride transporter), member 7 |
| SLC6A19 | 1201.71 | 5p15 | solute carrier family 6 (neutral amino acid transporter), member 19 |
| ADAMTS16 | 5140.443 | 5p15 | ADAM metallopeptidase with thrombospondin type 1 motif, 16 |
| PAPD7 | 6714.718 | 5p15 | PAP associated domain containing 7 |
| TAS2R1 | 9629.109 | 5p15 | taste receptor, type 2, member 1 |
| ROPN1L | 10441.974 | 5p15 | rhophilin associated tail protein 1 -like |
| FBXL7 | 15501.547 | 5p15.1 | F-box and leucine-rich repeat protein 7 |
| FAM134B | 16473.147 | 5p15.1 | family with sequence similarity 134, member B |
| BASP1 | 17216.932 | 5p15.1 | brain abundant, membrane attached signal protein 1 |
| RXFP3 | 33936.491 | 5p15.1-p14 | relaxin/insulin-like family peptide receptor 3 |
| MYO10 | 16662.016 | 5p15.1-p14.3 | myosin X |
| ZDHHC11 | 795.72 | 5p15.2 | zinc finger, DHHC-type containing 11 |
| SEMA5A | 9035.138 | 5p15.2 | sema domain, seven thrombospondin repeats (type 1 and type 1-like), |
| | | | transmembrane domain (TM) and short cytoplasmic domain, |
| | | | (semaphorin) 5A |
| FAM173B | 10225.62 | 5p15.2 | family with sequence similarity 173, member B |
| CCT5 | 10250.282 | 5p15.2 | chaperonin containing TCP1, subunit 5 (epsilon) |
| CMBL | 10277.707 | 5p15.2 | carboxymethylenebutenolidase homolog (Pseudomonas) |
| MARCH6 | 10353.751 | 5p15.2 | membrane-associated ring finger (C3HC4) 6, E3 ubiquitin protein ligase |
| DAP | 10679.342 | 5p15.2 | death-associated protein |
| CTNND2 | 10971.954 | 5p15.2 | catenin (cadherin-associated protein), delta 2 |
| ANKH | 14704.909 | 5p15.2 | ANKH inorganic pyrophosphate transport regulator |
| ZFR | 32354.456 | 5p15.2 | zinc finger RNA binding protein |
| CENPH | 68485.375 | 5p15.2 | centromere protein H |
| DDX4 | 55033.845 | 5p15.2-p13.1 | DEAD (Asp-Glu-Ala-Asp) box polypeptide 4 |
| SLC9A3 | 473.334 | 5p15.3 | solute carrier family 9, subfamily A (NHE3, cation proton antiporter 3), |
| | | | member 3 |
| NKD2 | 1009.077 | 5p15.3 | naked cuticle homolog 2 (Drosophila) |
| SLC6A3 | 1392.905 | 5p15.3 | solute carrier family 6 (neurotransmitter transporter), member 3 |
| MRPL36 | 1798.499 | 5p15.3 | mitochondrial ribosomal protein L36 |
| ADCY2 | 7396.343 | 5p15.3 | adenylate cyclase 2 (brain) |
| UBE2QL1 | 6448.736 | 5p15.31 | ubiquitin-conjugating enzyme E2Q family-like 1 |
| SRD5A1 | 6633.5 | 5p15.31 | steroid-5-alpha-reductase, alpha polypeptide 1 (3-oxo-5 alpha-steroid |
| | | | delta 4-dehydrogenase alpha 1) |
| MTRR | 7869.217 | 5p15.31 | 5-methyltetrahydro folate-homocysteine methyltransferase reductase |
| NSUN2 | 6599.352 | 5p15.32 | NOP2/Sun RNA methyltransferase family, member 2 |
| PLEKHG4B | 140.373 | 5p15.33 | pleckstrin homology domain containing, family G (with RhoGef |
| | | | domain) member 4B |
| CCDC127 | 204.875 | 5p15.33 | coiled-coil domain containing 127 |
| PDCD6 | 271.736 | 5p15.33 | programmed cell death 6 |
| AHRR | 304.291 | 5p15.33 | aryl-hydrocarbon receptor repressor |
| EXOC3 | 443.334 | 5p15.33 | exocyst complex component 3 |
| TPPP | 659.977 | 5p15.33 | tubulin polymerization promoting protein |
| TERT | 1253.287 | 5p15.33 | telomerase reverse transcriptase |
| CLPTM1L | 1318 | 5p15.33 | CLPTM1-like |
| LPCAT1 | 1461.542 | 5p15.33 | lysophosphatidylcholine acyltransferase 1 |
| NDUFS6 | 1801.496 | 5p15.33 | NADH dehydrogenase (ubiquinone) Fe—S protein 6, 13 kDa (NADH- |
| | | | coenzyme Q reductase) |
| IRX4 | 1877.541 | 5p15.33 | iroquois homeobox 4 |
| IRX2 | 2746.279 | 5p15.33 | iroquois homeobox 2 |
| IRX1 | 3596.168 | 5p15.33 | iroquois homeobox 1 |
| MTMR12 | 32227.111 | 5p15.33 | myotubularin related protein 12 |
| GTF2H2C_2 | 68856.074 | 5q | GTF2H2 family member C, copy 2 |
| IQGAP2 | 75843.234 | 5q | IQ motif containing GTPase activating protein 2 |
| CRHBP | 76248.68 | 5q | corticotropin releasing hormone binding protein |
| PAM | 102201.527 | 5q | peptidylglycine alpha-amidating monooxygenase |
| MRPS30 | 44809.027 | 5q11 | mitochondrial ribosomal protein S30 |
| ESM1 | 54273.695 | 5q11 | endothelial cell-specific molecule 1 |
| PPAP2A | 54720.67 | 5q11 | phosphatidic acid phosphatase type 2A |
| GZMA | 54398.474 | 5q11-q12 | granzyme A (granzyme 1, cytotoxic T-lymphocyte-associated serine |
| | | | esterase 3) |
| MSH3 | 79950.467 | 5q11-q12 | mutS homolog 3 |
| EMB | 49692.031 | 5q11.1 | embigin |
| ITGA1 | 52084.136 | 5q11.1 | integrin, alpha 1 |
| NDUFS4 | 52856.465 | 5q11.1 | NADH dehydrogenase (ubiquinone) Fe-S protein 4, 18 kDa (NADH- |
| | | | coenzyme Q reductase) |
| DROSHA | 31400.602 | 5q11.2 | drosha, ribonuclease type III |
| ISL1 | 50678.958 | 5q11.2 | ISL LIM homeobox 1 |
| ITGA2 | 52285.156 | 5q11.2 | integrin, alpha 2 (CD49B, alpha 2 subunit of VLA-2 receptor) |
| FST | 52776.264 | 5q11.2 | follistatin |
| HSPB3 | 53751.431 | 5q11.2 | heat shock 27 kDa protein 3 |
| MIR449A | 54466.36 | 5q11.2 | microRNA 449a |
| MIR449B | 54466.474 | 5q11.2 | microRNA 449b |
| CCNO | 54526.981 | 5q11.2 | cyclin O |
| DHX29 | 54552.073 | 5q11.2 | DEAH (Asp-Glu-Ala-His) box polypeptide 29 |
| SKIV2L2 | 54603.576 | 5q11.2 | superkiller viralicidic activity 2-like 2 (S. cerevisiae) |
| IL31RA | 55149.15 | 5q11.2 | interleukin 31 receptor A |
| IL6ST | 55230.925 | 5q11.2 | interleukin 6 signal transducer |
| MAP3K1 | 56110.9 | 5q11.2 | mitogen-activated protein kinase kinase kinase 1, E3 ubiquitin protein |
| | | | ligase |
| MIER3 | 56215.429 | 5q11.2 | mesoderm induction early response 1, family member 3 |
| HTR1A | 63255.875 | 5q11.2-q13 | 5-hydroxytryptamine (serotonin) receptor 1A, G protein-coupled |
| TAF9 | 68660.57 | 5q11.2-q13.1 | TAF9 RNA polymerase II, TATA box binding protein (TBP)-associated |
| | | | factor, 32 kDa |
| DHFR | 79922.045 | 5q11.2-q13.2 | dihydrofolate reductase |
| PDE4D | 58264.866 | 5q12 | phosphodiesterase 4D, cAMP-specific |
| DEPDC1B | 59892.739 | 5q12 | DEP domain containing 1B |
| ELOVL7 | 60047.616 | 5q12 | ELOVL fatty acid elongase 7 |
| CD180 | 66478.104 | 5q12 | CD180 molecule |
| CCNB1 | 68462.837 | 5q12 | cyclin B1 |
| KIF2A | 61601.989 | 5q12-q13 | kinesin heavy chain member 2A |
| FOXD1 | 72742.085 | 5q12-q13 | forkheadbox D1 |
| PART1 | 59819.923 | 5q12.1 | prostate androgen-regulated transcript 1 (non-protein coding) |
| ERCC8 | 60169.659 | 5q12.1 | excision repair cross-complementation group 8 |
| NDUFAF2 | 60240.956 | 5q12.1 | NADH dehydrogenase (ubiquinone) complex I, assembly factor 2 |
| IPO11 | 61714.711 | 5q12.1 | importin 11 |
| CDK7 | 68530.622 | 5q12.1 | cyclin-dependent kinase 7 |
| PLK2 | 57749.81 | 5q12.1-q13.2 | polo-like kinase 2 |
| RNF180 | 63461.671 | 5q12.3 | ring finger protein 180 |
| CWC27 | 64064.755 | 5q12.3 | CWC27 spliceosome-associated protein homolog (S. cerevisiae) |
| CENPK | 64813.593 | 5q12.3 | centromere protein K |
| TRIM23 | 64885.507 | 5q12.3 | tripartite motif containing 23 |
| SGTB | 64961.755 | 5q12.3 | small glutamine-rich tetratricopeptide repeat (TPR)-containing, beta |
| MAST4 | 65892.176 | 5q12.3 | microtubule associated serine/threonine kinase family member 4 |
| RAB3C | 57878.939 | 5q13 | RAB3C, member RAS oncogene family |
| ADAMTS6 | 64444.563 | 5q13 | ADAM metallopeptidase with thrombospondin type 1 motif, 6 |
| RAD17 | 68665.887 | 5q13 | RAD17 homolog (S. pombe) |
| MAP1B | 71403.118 | 5q13 | micro tubule-associated protein 1B |
| ENC1 | 73923.231 | 5q13 | ectodermal-neural cortex 1 (with BTB domain) |
| POLK | 74807.657 | 5q13 | polymerase (DNA directed) kappa |
| SV2C | 75379.239 | 5q13 | synaptic vesicle glycoprotein 2C |
| F2RL2 | 75911.307 | 5q13 | coagulation factor II (thrombin) receptor-like 2 |
| F2R | 76011.868 | 5q13 | coagulation factor II (thrombin) receptor |
| F2RL1 | 76114.833 | 5q13 | coagulation factor II (thrombin) receptor-like 1 |
| BHMT2 | 78365.547 | 5q13 | betaine--homocysteine S-methyltransferase 2 |
| THBS4 | 79331.17 | 5q13 | thrombospondin 4 |
| RASGRF2 | 80256.508 | 5q13 | Ras protein-specific guanine nucleotide-releasing factor 2 |
| RASA1 | 86564.07 | 5q13 | RAS p21 protein activator (GTPase activating protein) 1 |
| CHSY3 | 129240.523 | 5q13 | chondroitin sulfate synthase 3 |
| PIK3R1 | 67588.396 | 5q13.1 | phosphoinositide-3-kinase, regulatory subunit 1 (alpha) |
| MARVELD2 | 68710.939 | 5q13.1 | MARVEL domain containing 2 |
| OCLN | 68788.59 | 5q13.1 | occludin |
| TNPO1 | 72112.418 | 5q13.1 | transportin 1 |
| GTF2H2C | 68856.051 | 5q13.2 | GTF2H2 family member C |
| NAIP | 70264.31 | 5q13.2 | NLR family, apoptosis inhibitory protein |
| GTF2H2 | 70330.951 | 5q13.2 | general transcription factor IIH, polypeptide 2, 44 kDa |
| GTF2H2B | 70331.332 | 5q13.2 | general transcription factor IIH, polypeptide 2B (pseudogene) |
| CARTPT | 71014.99 | 5q13.2 | CART prepropeptide |
| FCHO2 | 72251.808 | 5q13.2 | FCH domain only 2 |
| UTP15 | 72861.566 | 5q13.2 | UTP15, U3 small nucleolar ribonucleoprotein, homolog (S. cerevisiae) |
| ARHGEF28 | 72921.983 | 5q13.2 | Rho guanine nucleotide exchange factor (GEF) 28 |
| ZNF366 | 71739.234 | 5q13.2\|5q13.2 | zinc finger protein 366 |
| BTF3 | 72794.25 | 5q13.3 | basic transcription factor 3 |
| HEXB | 73980.969 | 5q13.3 | hexosaminidase B (beta polypeptide) |
| COL4A3BP | 74666.928 | 5q13.3 | collagen, type IV, alpha 3 (Goodpasture antigen) binding protein |
| HMGCR | 74632.993 | 5q13.3-q14 | 3-hydroxy-3-methylglutaryl-CoA reductase |
| CCNH | 86690.079 | 5q13.3-q14 | cyclin H |
| EDIL3 | 83236.414 | 5q14 | EGF-like repeats and discoidin I-like domains 3 |
| NR2F1 | 92919.043 | 5q14 | nuclear receptor subfamily 2, group F, member 1 |
| GLRX | 95149.553 | 5q14 | glutaredoxin (thioltransferase) |
| RIOK2 | 96502.45 | 5q14 | RIO kinase 2 |
| PDE8B | 76506.706 | 5q14.1 | phosphodiesterase 8B |
| OTP | 76924.537 | 5q14.1 | orthopedia homeobox |
| SCAMPI | 77656.327 | 5q14.1 | secretory carrier membrane protein 1 |
| ARSB | 78073.037 | 5q14.1 | arylsulfatase B |
| DMGDH | 78293.387 | 5q14.1 | dimethylglycine dehydrogenase |
| BHMT | 78407.604 | 5q14.1 | betaine--homocysteine S-methyltransferase |
| JMY | 78531.925 | 5q14.1 | junction mediating and regulatory protein, p53 cofactor |
| FAM151B | 79783.8 | 5q14.1 | family with sequence similarity 151, member B |
| SSBP2 | 80713.179 | 5q14.1 | single-stranded DNA binding protein 2 |
| ATG10 | 81267.844 | 5q14.1 | autophagy related 10 |
| XRCC4 | 82373.317 | 5q14.2 | X-ray repair complementing defective repair in Chinese hamster cells 4 |
| VCAN | 82767.493 | 5q14.2-q14.3 | versican |
| HAPLN1 | 82934.017 | 5q14.3 | hyaluronan and proteoglycan link protein 1 |
| MIR9-2 | 87962.671 | 5q14.3 | microRNA 9-2 |
| MEF2C | 88014.058 | 5q14.3 | myocyte enhancer factor 2C |
| LUCAT1 | 90598.803 | 5q14.3 | lung cancer associated transcript 1 (non-protein coding) |
| ARRDC3 | 90664.541 | 5q14.3 | arrestin domain containing 3 |
| FAM172A | 92953.431 | 5q15 | family with sequence similarity 172, member A |
| RHOBTB3 | 95066.85 | 5q15 | Rho-related BTB domain containing 3 |
| ELL2 | 95220.802 | 5q15 | elongation factor, RNA polymerase II, 2 |
| CAST | 95997.861 | 5q15 | calpastatin |
| ERAP1 | 96110.188 | 5q15 | endoplasmic reticulum aminopeptidase 1 |
| ERAP2 | 96211.644 | 5q15 | endoplasmic reticulum aminopeptidase 2 |
| LNPEP | 96271.346 | 5q15 | leucyl/cystinyl aminopeptidase |
| PCSK1 | 95726.04 | 5q15-q21 | proprotein convertase subtilisin/kexin type 1 |
| CHD1 | 98190.908 | 5q15-q21 | chromodomain helicase DNA binding protein 1 |
| HSD17B4 | 118788.138 | 5q2 | hydroxysteroid (17-beta) dehydrogenase 4 |
| ST8SIA4 | 100142.639 | 5q21 | ST8 alpha-N-acetyl-neuraminide alpha-2,8-sialyltransferase 4 |
| EFNA5 | 106712.59 | 5q21 | ephrin-A5 |
| FER | 108083.523 | 5q21 | fer (fps/fes related) tyrosine kinase |
| CPEB4 | 173315.331 | 5q21 | cytoplasmic polyadenylation element binding protein 4 |
| APC | 112073.556 | 5q21-q22 | adenomatous polyposis coli |
| MCC | 112357.796 | 5q21-q22 | mutated in colorectal cancers |
| ATG12 | 115163.894 | 5q21-q22 | autophagy related 12 |
| RGMB | 98104.999 | 5q21.1 | repulsive guidance molecule family member b |
| GIN1 | 102421.704 | 5q21.1 | gypsy retrotransposon integrase 1 |
| RAB9BP1 | 104435.175 | 5q21.2 | RAB9B, member RAS oncogene family pseudogene 1 |
| FBXL17 | 107194.734 | 5q21.3 | F-box and leucine-rich repeat protein 17 |
| STARD4 | 110834.022 | 5q22 | StAR-related lipid transfer (START) domain containing 4 |
| TRIM36 | 114506.799 | 5q22 | tripartite motif containing 36 |
| AP3S1 | 115177.619 | 5q22 | adaptor-related protein complex 3, sigma 1 subunit |
| PJA2 | 108670.41 | 5q22.1 | praja ring finger 2, E3 ubiquitin protein ligase |
| TSLP | 110407.39 | 5q22.1 | thymic stromal lymphopoietin |
| CAMK4 | 110559.947 | 5q22.1 | calcium/calmodulin-dependent protein kinase IV |
| RNU2-1 | 110820.971 | 5q22.1 | RNA, U2 small nuclear 1 |
| NREP | 111065 | 5q22.1 | neuronal regeneration related protein |
| ZRSR1 | 112227.309 | 5q22.2 | zinc finger (CCCH type), RNA-binding motif and serine/arginine rich 1 |
| YTHDC2 | 112849.391 | 5q22.3 | YTH domain containing 2 |
| CSNK1G3 | 122847.793 | 5q23 | casein kinase 1, gamma 3 |
| CAMLG | 134074.17 | 5q23 | calcium modulating ligand |
| HBEGF | 139712.428 | 5q23 | heparin-binding EGF-like growth factor |
| GPX3 | 150399.999 | 5q23 | glutathione peroxidase 3 (plasma) |
| FBN2 | 127593.601 | 5q23-q31 | fibrillin 2 |
| IL3 | 131396.347 | 5q23-q31 | interleukin 3 |
| CSF2 | 131409.485 | 5q23-q31 | colony stimulating factor 2 (granulocyte-macrophage) |
| IRF1 | 131817.301 | 5q23-q31 | interferon regulatory factor 1 |
| IL5 | 131877.136 | 5q23-q31 | interleukin 5 |
| RAD50 | 131892.616 | 5q23-q31 | RAD50 homolog (S. cerevisiae) |
| IL4 | 132009.678 | 5q23-q31 | interleukin 4 |
| NEUROG1 | 134869.972 | 5q23-q31 | neurogenin 1 |
| EGR1 | 137801.181 | 5q23-q31 | early growth response 1 |
| NRG2 | 139226.364 | 5q23-q33 | neuregulin 2 |
| FABP6 | 159626.048 | 5q23-q35 | fatty acid binding protein 6, ileal |
| PGGT1B | 114546.527 | 5q23.1 | protein geranylgeranyltransferase type I, beta subunit |
| TNFAIP8 | 118604.387 | 5q23.1 | tumor necrosis factor, alpha-induced protein 8 |
| FTMT | 121187.65 | 5q23.1 | ferritin mitochondrial |
| CDO1 | 115140.43 | 5q23.2 | cysteine dioxygenase type 1 |
| SNCAIP | 121647.82 | 5q23.2 | synuclein, alpha interacting protein |
| SNX2 | 122110.691 | 5q23.2 | sorting nexin 2 |
| LMNB1 | 126112.845 | 5q23.2 | lamin B1 |
| PRRC1 | 126853.301 | 5q23.2 | proline-rich coiled-coil 1 |
| SLC12A2 | 127419.483 | 5q23.3 | solute carrier family 12 (sodium/potassium/chloride transporter), |
| | | | member 2 |
| SLC27A6 | 128301.213 | 5q23.3 | solute carrier family 27 (fatty acid transporter), member 6 |
| ADAMTS19 | 128796.103 | 5q23.3 | ADAM metallopeptidase with thrombospondin type 1 motif, 19 |
| FNIP1 | 130977.407 | 5q23.3 | folliculin interacting protein 1 |
| SLC22A4 | 131630.145 | 5q23.3 | solute carrier family 22 (organic cation/zwitterion transporter), member |
| | | | 4 |
| SLC22A5 | 131705.401 | 5q23.3 | solute carrier family 22 (organic cation/carnitine transporter), member 5 |
| RNF14 | 141346.402 | 5q23.3-q31.1 | ring finger protein 14 |
| LOX | 121398.89 | 5q23.3-q31.2 | lysyl oxidase |
| SEPP1 | 42799.982 | 5q31 | selenoprotein P, plasma, 1 |
| ALDH7A1 | 125877.533 | 5q31 | aldehyde dehydrogenase 7 family, member A1 |
| ACSL6 | 131285.667 | 5q31 | acyl-CoA synthetase long-chain family member 6 |
| P4HA2 | 131528.304 | 5q31 | prolyl 4-hydroxylase, alpha polypeptide II |
| IL13 | 131993.865 | 5q31 | interleukin 13 |
| SEPT8 | 132086.509 | 5q31 | septin 8 |
| AFF4 | 132211.071 | 5q31 | AF4/FMR2 family, member 4 |
| VDAC1 | 133307.566 | 5q31 | voltage-dependent anion channel 1 |
| TCF7 | 133451.298 | 5q31 | transcription factor 7 (T-cell specific, HMG-box) |
| SKP1 | 133492.082 | 5q31 | S-phase kinase-associated protein 1 |
| CDKL3 | 133634.115 | 5q31 | cyclin-dependent kinase-like 3 |
| CXCL14 | 134906.371 | 5q31 | chemokine (C-X-C motif) ligand 14 |
| FBXL21 | 135266.006 | 5q31 | F-box and leucine-rich repeat protein 21 (gene/pseudogene) |
| TGFBI | 135364.584 | 5q31 | transforming growth factor, beta-induced, 68 kDa |
| SMAD5 | 135468.534 | 5q31 | SMAD family member 5 |
| WNT8A | 137419.774 | 5q31 | wingless-type MMTV integration site family, member 8A |
| BRD8 | 137492.573 | 5q31 | bromodomain containing 8 |
| KIF20A | 137514.417 | 5q31 | kinesin family member 20A |
| CDC23 | 137523.337 | 5q31 | cell division cycle 23 |
| CDC25C | 137620.954 | 5q31 | cell division cycle 25C |
| KDM3B | 137688.285 | 5q31 | lysine (K)-specific demethylase 3B |
| PURA | 139493.708 | 5q31 | purine-rich element binding protein A |
| SRA1 | 139929.652 | 5q31 | steroid receptor RNA activator 1 |
| ZMAT2 | 140080.032 | 5q31 | zinc finger, matrin-type 2 |
| PCDHA@ | 140165.876 | 5q31 | protocadherin alpha cluster, complex locus |
| PCDHB@ | 140430.979 | 5q31 | protocadherin beta cluster |
| TAF7 | 140698.057 | 5q31 | TAF7 RNA polymerase II, TATA box binding protein (TBP)-associated |
| | | | factor, 55 kDa |
| PCDHGA1 | 140710.252 | 5q31 | protocadherin gamma subfamily A, 1 |
| PCDHG@ | 140710.252 | 5q31 | protocadherin gamma cluster |
| PCDHGA5 | 140743.898 | 5q31 | protocadherin gamma subfamily A, 5 |
| PCDHGB6 | 140787.77 | 5q31 | protocadherin gamma subfamily B, 6 |
| PCDHGA11 | 140800.537 | 5q31 | protocadherin gamma subfamily A, 11 |
| PCDHGC3 | 140855.569 | 5q31 | protocadherin gamma subfamily C, 3 |
| DIAPH1 | 140894.588 | 5q31 | diaphanous-related formin 1 |
| ARHGAP26 | 142150.292 | 5q31 | Rho GTPase activating protein 26 |
| NR3C1 | 142657.496 | 5q31-q32 | nuclear receptor subfamily 3, group C, member 1 (glucocorticoid |
| | | | receptor) |
| SPINK5 | 147443.535 | 5q31-q32 | serine peptidase inhibitor, Kazal type 5 |
| ADRB2 | 148206.156 | 5q31-q32 | adrenoceptor beta 2, surface |
| ITK | 156607.907 | 5q31-q32 | IL2-inducible T-cell kinase |
| HTR4 | 147861.115 | 5q31-q33 | 5-hydroxytryptamine (serotonin) receptor 4, G protein-coupled |
| SPARC | 151040.657 | 5q31-q33 | secreted protein, acidic, cysteine-rich (osteonectin) |
| CNOT8 | 154238.198 | 5q31-q33 | CCR4-NOT transcription complex, subunit 8 |
| SLC26A2 | 149340.3 | 5q31-q34 | solute carrier family 26 (anion exchanger), member 2 |
| IL9 | 135227.935 | 5q31-q35 | interleukin 9 |
| CDC42SE2 | 130599.702 | 5q31.1 | CDC42 small effector 2 |
| RAPGEF6 | 130759.614 | 5q31.1 | Rap guanine nucleotide exchange factor (GEF) 6 |
| PDLIM4 | 131593.351 | 5q31.1 | PDZ and LIM domain 4 |
| GDF9 | 132196.874 | 5q31.1 | growth differentiation factor 9 |
| LEAP2 | 132209.358 | 5q31.1 | liver expressed antimicrobial peptide 2 |
| HSPA4 | 132387.662 | 5q31.1 | heat shock 70 kDa protein 4 |
| SKP1P1 | 133493.426 | 5q31.1 | S-phase kinase-associated protein 1 pseudogene 1 |
| PPP2CA | 133532.148 | 5q31.1 | protein phosphatase 2, catalytic subunit, alpha isozyme |
| UBE2B | 133706.867 | 5q31.1 | ubiquitin-conjugating enzyme E2B |
| DDX46 | 134094.461 | 5q31.1 | DEAD (Asp-Glu-Ala-Asp) box polypeptide 46 |
| TXNDC15 | 134209.46 | 5q31.1 | thioredoxin domain containing 15 |
| PITX1 | 134363.424 | 5q31.1 | paired-like homeodomain 1 |
| H2AFY | 134670.071 | 5q31.1 | H2A histone family, member Y |
| LECT2 | 135282.6 | 5q31.1 | leukocyte cell-derived chemotaxin 2 |
| VTRNA2-1 | 135416.187 | 5q31.1 | vault RNA 2-1 |
| HSPA9 | 137890.571 | 5q31.1 | heat shock 70 kDa protein 9 (mortalin) |
| GRIA1 | 152870.084 | 5q31.1 | glutamate receptor, ionotropic, AMPA 1 |
| HDAC3 | 141000.443 | 5q31.1-q31.2 | histone deacetylase 3 |
| GFRA3 | 137588.069 | 5q31.1-q31.3 | GDNF family receptor alpha 3 |
| IL12B | 158741.791 | 5q31.1-q33.1 | interleukin 12B |
| HINT1 | 130494.976 | 5q31.2 | histidine triad nucleotide binding protein 1 |
| JADE2 | 133861.34 | 5q31.2 | jade family PHD finger 2 |
| SPOCK1 | 136310.987 | 5q31.2 | sparc/osteonectin, cwcv and kazal-like domains proteoglycan (testican) |
| | | | 1 |
| MIR874 | 136983.261 | 5q31.2 | microRNA 874 |
| MYOT | 137203.545 | 5q31.2 | myotilin |
| NME5 | 137450.861 | 5q31.2 | NME/NM23 family member 5 |
| ETF1 | 137841.782 | 5q31.2 | eukaryotic translation termination factor 1 |
| CTNNA1 | 138089.085 | 5q31.2 | catenin (cadherin-associated protein), alpha 1, 102 kDa |
| SLC23A1 | 138702.885 | 5q31.2 | solute carrier family 23 (ascorbic acid transporter), member 1 |
| MZB1 | 138723.257 | 5q31.2 | marginal zone B and B1 cell-specific protein |
| DNAJC18 | 138745.892 | 5q31.2 | DnaJ (Hsp40) homolog, subfamily C, member 18 |
| TMEM173 | 138855.113 | 5q31.2 | transmembrane protein 173 |
| MATR3 | 138609.441 | 5q31.3 | matrin 3 |
| UBE2D2 | 138940.751 | 5q31.3 | ubiquitin-conjugating enzyme E2D 2 |
| CXXC5 | 139028.301 | 5q31.3 | CXXC finger protein 5 |
| PSD2 | 139175.406 | 5q31.3 | pleckstrin and Sec7 domain containing 2 |
| ANKHD1 | 139781.399 | 5q31.3 | ankyrin repeat and KH domain containing 1 |
| EIF4EBP3 | 139927.251 | 5q31.3 | eukaryotic translation initiation factor 4E binding protein 3 |
| CD14 | 140011.313 | 5q31.3 | CD14 molecule |
| IK | 140027.384 | 5q31.3 | IK cytokine, down-regulator of HLA II |
| WDR55 | 140044.384 | 5q31.3 | WD repeat domain 55 |
| DND1 | 140050.381 | 5q31.3 | DND microRNA-mediated repression inhibitor 1 |
| HARS | 140053.489 | 5q31.3 | histidyl-tRNA synthetase |
| HARS2 | 140071.011 | 5q31.3 | histidyl-tRNA synthetase 2, mitochondrial |
| FCHSD1 | 141018.869 | 5q31.3 | FCH and double SH3 domains 1 |
| ARAP3 | 141032.968 | 5q31.3 | ArfGAP with RhoGAP domain, ankyrin repeat and PH domain 3 |
| PCDH1 | 141242.217 | 5q31.3 | protocadherin 1 |
| SPRY4 | 141689.992 | 5q31.3 | sprouty homolog 4 (Drosophila) |
| SPRY4-IT1 | 141697.199 | 5q31.3 | SPRY4 intronic transcript 1 (non-protein coding) |
| YIPF5 | 143537.723 | 5q31.3 | Yip1 domain family, member 5 |
| FGF1 | 141971.743 | 5q31.3-q33.2 | fibroblast growth factor 1 (acidic) |
| HMHB1 | 143191.726 | 5q32 | histocompatibility (minor) HB-1 |
| PRELID2 | 145135.907 | 5q32 | PRELI domain containing 2 |
| LARS | 145492.589 | 5q32 | leucyl-tRNA synthetase |
| POU4F3 | 145718.587 | 5q32 | POU class 4 homeobox 3 |
| PPP2R2B | 145969.067 | 5q32 | protein phosphatase 2, regulatory subunit B, beta |
| DPYSL3 | 146770.371 | 5q32 | dihydropyrimidinase-like 3 |
| JAKMIP2 | 146965.004 | 5q32 | janus kinase and microtubule interacting protein 2 |
| SPINK1 | 147204.143 | 5q32 | serine peptidase inhibitor, Kazal type 1 |
| SCGB3A2 | 147258.274 | 5q32 | secretoglobin, family 3A, member 2 |
| SPINK7 | 147691.99 | 5q32 | serine peptidase inhibitor, Kazal type 7 (putative) |
| MIR143 | 148808.481 | 5q32 | microRNA 143 |
| MIR145 | 148810.209 | 5q32 | microRNA 145 |
| CSNK1A1 | 148875.457 | 5q32 | casein kinase 1, alpha 1 |
| CSF1R | 149432.854 | 5q32 | colony stimulating factor 1 receptor |
| CDX1 | 149546.344 | 5q32 | caudal type homeobox 1 |
| CD74 | 149781.2 | 5q32 | CD74 molecule, major histocompatibility complex, class II invariant |
| | | | chain |
| ATOX1 | 151122.383 | 5q32 | antioxidant 1 copper chaperone |
| TNIP1 | 150409.504 | 5q32-q33.1 | TNFAIP3 interacting protein 1 |
| CCNG1 | 162864.577 | 5q32-q34 | cyclin G1 |
| BNIP1 | 172571.445 | 5q33-q34 | BCL2/adenovirus E1B 19 kDa interacting protein 1 |
| FGFR4 | 176513.873 | 5q33-qter | fibroblast growth factor receptor 4 |
| FBXO38 | 147763.498 | 5q33.1 | F-box protein 38 |
| AFAP1L1 | 148651.401 | 5q33.1 | actin filament associated protein 1 -like 1 |
| IL17B | 148753.83 | 5q33.1 | interleukin 17B |
| PPARGC1B | 149109.815 | 5q33.1 | peroxisome proliferator-activated receptor gamma, coactivator 1 beta |
| HMGXB3 | 149380.169 | 5q33.1 | HMG box domain containing 3 |
| PDGFRB | 149493.402 | 5q33.1 | platelet-derived growth factor receptor, beta polypeptide |
| NDST1 | 149887.674 | 5q33.1 | N-deacetylase/N-sulfotransferase (heparan glucosaminyl) 1 |
| RBM22 | 150070.352 | 5q33.1 | RNA binding motif protein 22 |
| IRGM | 150226.085 | 5q33.1 | immunity-related GTPase family, M |
| ZNF300 | 150273.954 | 5q33.1 | zinc finger protein 300 |
| ANXA6 | 150480.267 | 5q33.1 | annexin A6 |
| SLC36A1 | 150827.163 | 5q33.1 | solute carrier family 36 (proton/amino acid symporter), member 1 |
| FAT2 | 150883.653 | 5q33.1 | FAT atypical cadherin 2 |
| G3BP1 | 151151.476 | 5q33.1 | GTPase activating protein (SH3 domain) binding protein 1 |
| MYOZ3 | 150040.403 | 5q33.2 | myozenin 3 |
| SAP30L | 153825.517 | 5q33.2 | SAP30-like |
| HAVCR1 | 156456.531 | 5q33.2 | hepatitis A virus cellular receptor 1 |
| HMMR | 162887.517 | 5q33.2-qter | hyaluronan-mediated motility receptor (RHAMM) |
| TIMD4 | 156346.293 | 5q33.3 | T-cell immunoglobulin and mucin domain containing 4 |
| ADAM19 | 156904.312 | 5q33.3 | ADAM metallopeptidase domain 19 |
| UBLCP1 | 158690.089 | 5q33.3 | ubiquitin-like domain containing CTD phosphatase 1 |
| ADRA1B | 159343.74 | 5q33.3 | adrenoceptor alpha 1B |
| CCNJL | 159678.671 | 5q33.3 | cyclin J-like |
| C1QTNF2 | 159774.775 | 5q33.3 | C1q and tumor necrosis factor related protein 2 |
| SLU7 | 159828.648 | 5q33.3 | SLU7 splicing factor homolog (S. cerevisiae) |
| GEMIN5 | 154266.976 | 5q34 | gem (nuclear organelle) associated protein 5 |
| HAVCR2 | 156512.843 | 5q34 | hepatitis A virus cellular receptor 2 |
| CYFIP2 | 156693.09 | 5q34 | cytoplasmic FMR1 interacting protein 2 |
| EBF1 | 158122.923 | 5q34 | early B-cell factor 1 |
| MIR146A | 159912.359 | 5q34 | microRNA 146a |
| ATP10B | 159990.127 | 5q34 | ATPase, class V, type 10B |
| GABRB2 | 160715.436 | 5q34 | gamma-aminobutyric acid (GABA) A receptor, beta 2 |
| GABRA1 | 161274.197 | 5q34 | gamma-aminobutyric acid (GABA) A receptor, alpha 1 |
| WWC1 | 167719.065 | 5q34 | WW and C2 domain containing 1 |
| MIR218-2 | 168195.151 | 5q34 | microRNA 218-2 |
| FOXI1 | 169532.917 | 5q34 | forkheadbox 11 |
| KCNMB1 | 169805.165 | 5q34 | potassium large conductance calcium-activated channel, subfamily M, |
| | | | beta member 1 |
| RANBP17 | 170288.896 | 5q34 | RAN binding protein 17 |
| FGF18 | 170846.667 | 5q34 | fibroblast growth factor 18 |
| NKX2-5 | 172659.107 | 5q34 | NK2 homeobox 5 |
| MAT2B | 162932.534 | 5q34-q35 | methionine adenosyltransferase II, beta |
| FLT4 | 180034.753 | 5q34-q35 | fms-related tyrosine kinase 4 |
| ADAMTS12 | 33527.287 | 5q35 | ADAM metallopeptidase with thrombospondin type 1 motif, 12 |
| TENM2 | 166711.843 | 5q35 | teneurin transmembrane protein 2 |
| SLIT3 | 168088.738 | 5q35 | slit homolog 3 (Drosophila) |
| SNCB | 176047.21 | 5q35 | synuclein, beta |
| NSD1 | 176560.833 | 5q35 | nuclear receptor binding SET domain protein 1 |
| LMAN2 | 176758.563 | 5q35 | lectin, mannose-binding 2 |
| GRK6 | 176853.687 | 5q35 | G protein-coupled receptor kinase 6 |
| DOK3 | 176928.906 | 5q35 | docking protein 3 |
| FAM193B | 176946.79 | 5q35 | family with sequence similarity 193, member B |
| CLK4 | 178029.665 | 5q35 | CDC-like kinase 4 |
| CANX | 179125.93 | 5q35 | calnexin |
| MAML1 | 179159.851 | 5q35 | mastermind-like 1 (Drosophila) |
| LTC4S | 179220.986 | 5q35 | leukotriene C4 synthase |
| MGAT4B | 179224.598 | 5q35 | mannosyl (alpha-1,3-)-glycoprotein beta-1,4-N- |
| | | | acetylglucosaminyltransferase, isozyme B |
| SQSTM1 | 179233.388 | 5q35 | sequestosome 1 |
| MAPK9 | 179673.028 | 5q35 | mitogen-activated protein kinase 9 |
| SCGB3A1 | 180017.105 | 5q35-qter | secretoglobin, family 3A, member 1 |
| PTTG1 | 159848.917 | 5q35.1 | pituitary tumor-transforming 1 |
| RARS | 167913.463 | 5q35.1 | arginyl-tRNA synthetase |
| DOCK2 | 169064.251 | 5q35.1 | dedicator of cytokinesis 2 |
| LCP2 | 169675.088 | 5q35.1 | lymphocyte cytosolic protein 2 (SH2 domain containing leukocyte |
| | | | protein of 76 kDa) |
| KCNIP1 | 169931.04 | 5q35.1 | Kv channel interacting protein 1 |
| GABRP | 170210.723 | 5q35.1 | gamma-aminobutyric acid (GABA) A receptor, pi |
| TLX3 | 170736.288 | 5q35.1 | T-cell leukemia homeobox 3 |
| NPM1 | 170814.708 | 5q35.1 | nucleophosmin (nucleolar phosphoprotein B23, numatrin) |
| STK10 | 171469.074 | 5q35.1 | serine/threonine kinase 10 |
| DUSP1 | 172195.093 | 5q35.1 | dual specificity phosphatase 1 |
| ERGIC1 | 172261.223 | 5q35.1 | endoplasmic reticulum-golgi intermediate compartment (ERGIC) 1 |
| B4GALT7 | 177027.119 | 5q35.1-q35.3 | xylosylprotein beta 1,4-galactosyltransferase, polypeptide 7 |
| ATP6V0E1 | 172410.763 | 5q35.2 | ATPase, H+ transporting, lysosomal 9 kDa, V0 subunit e1 |
| STC2 | 172741.726 | 5q35.2 | stanniocalcin 2 |
| C5orf47 | 173416.162 | 5q35.2 | chromosome 5 open reading frame 47 |
| MSX2 | 174151.575 | 5q35.2 | msh homeobox 2 |
| HRH2 | 175085.04 | 5q35.2 | histamine receptor H2 |
| NOP16 | 175810.94 | 5q35.2 | NOP16 nucleolar protein |
| CLTB | 175819.456 | 5q35.2 | clathrin, light chain B |
| CDHR2 | 175969.512 | 5q35.2 | cadherin-related family member 2 |
| HK3 | 176307.87 | 5q35.2 | hexokinase 3 (white cell) |
| UIMC1 | 176332.006 | 5q35.2 | ubiquitin interaction motif containing 1 |
| RNF44 | 175953.7 | 5q35.3 | ring finger protein 44 |
| UNC5A | 176237.56 | 5q35.3 | unc-5 homolog A (C. elegans) |
| RAB24 | 176728.191 | 5q35.3 | RAB24, member RAS oncogene family |
| RGS14 | 176784.844 | 5q35.3 | regulator of G-protein signaling 14 |
| DBN1 | 176883.614 | 5q35.3 | drebrin 1 |
| PDLIM7 | 176910.395 | 5q35.3 | PDZ and LIM domain 7 (enigma) |
| DDX41 | 176938.578 | 5q35.3 | DEAD (Asp-Glu-Ala-Asp) box polypeptide 41 |
| PROP1 | 177419.236 | 5q35.3 | PROP paired-like homeobox 1 |
| NHP2 | 177576.465 | 5q35.3 | NHP2 ribonucleoprotein |
| HNRNPAB | 177631.508 | 5q35.3 | heterogeneous nuclear ribonucleoprotein A/B |
| COL23A1 | 177664.617 | 5q35.3 | collagen, type XXIII, alpha 1 |
| HNRNPH1 | 179041.179 | 5q35.3 | heterogeneous nuclear ribonucleoprotein H1 (H) |
| TBC1D9B | 179289.071 | 5q35.3 | TBC1 domain family, member 9B (with GRAM domain) |
| RNF130 | 179382.067 | 5q35.3 | ring finger protein 130 |
| MIR340 | 179442.303 | 5q35.3 | microRNA 340 |
| RASGEF1C | 179527.795 | 5q35.3 | RasGEF domain family, member 1C |
| MGAT1 | 180217.541 | 5q35.3 | mannosyl (alpha-1,3-)-glycoprotein beta-1,2-N- |
| | | | acetylglucosaminyltransferase |
| HEIH | 180256.954 | 5q35.3 | hepatocellular carcinoma up-regulated EZH2-associated long non- |
| | | | coding RNA |
| GNB2L1 | 180663.928 | 5q35.3 | guanine nucleotide binding protein (G protein), beta polypeptide 2-like 1 |

*Information obtained from http://atlasgeneticsoncology.org/Indexbychrom/idxg_5.html (last accessed on Sep. 11, 2014)

TABLE 8
List of cancer genes on human chromosome 20 *

| | GoldenPath | | |
|---|---|---|---|
| Symbol | (Mb) | Location | Description |
| SSTR4 | 23016.057 | 20 | somatostatin receptor 4 |
| CHRNA4 | 61974.662 | 20 | cholinergic receptor, nicotinic, alpha 4 (neuronal) |
| ITPA | 3190.006 | 20p | inosine triphosphatase (nucleoside triphosphate pyrophosphatase) |
| SNX5 | 17922.24 | 20p11 | sorting nexin 5 |
| FOXA2 | 22561.642 | 20p11 | forkhead box A2 |
| MIR663A | 26188.822 | 20p11.1 | microRNA 663 a |
| PCSK2 | 17206.752 | 20p11.2 | proprotein convertase subtilisin/kexin type 2 |
| RBBP9 | 18467.188 | 20p11.2 | retinoblastoma binding protein 9 |
| CRNKL1 | 20015.005 | 20p11.2 | crooked neck pre-mRNA splicing factor 1 |
| INSM1 | 20348.765 | 20p11.2 | insulinoma-associated 1 |
| THBD | 23026.27 | 20p11.2 | thrombomodulin |
| CST3 | 23614.294 | 20p11.2 | cystatin C |
| CST1 | 23728.19 | 20p11.2 | cystatin SN |
| APMAP | 24943.58 | 20p11.2 | adipocyte plasma membrane associated protein |
| XRN2 | 21283.942 | 20p11.2-p11.1 | 5′-3′ exoribonuclease 2 |
| CD93 | 23059.993 | 20p11.21 | CD93 molecule |
| CST5 | 23856.572 | 20p11.21 | cystatin D |
| CST7 | 24929.866 | 20p11.21 | cystatin F (leukocystatin) |
| PYGB | 25228.706 | 20p11.21 | phosphorylase, glycogen; brain |
| GINS1 | 25388.323 | 20p11.21 | GINS complex subunit 1 (Psfl homolog) |
| RIN2 | 19867.165 | 20p11.22 | Ras and Rab interactor 2 |
| NKX2-2 | 21491.66 | 20p11.22 | NK2 homeobox 2 |
| PAX1 | 21686.297 | 20p11.22 | paired box 1 |
| NINL | 25433.338 | 20p11.22-p11.1 | ninein-like |
| MGME1 | 17949.762 | 20p11.23 | mitochondrial genome maintenance exonuclease 1 |
| DTD1 | 18568.556 | 20p11.23 | D-tyrosyl-tRNA deacylase 1 |
| NAA20 | 19997.934 | 20p11.23 | N(alpha)-acetyltransferase 20, NatB catalytic subunit |
| RALGAPA2 | 20370.272 | 20p11.23 | Ral GTPase activating protein, alpha subunit 2 (catalytic) |
| BMP2 | 6748.745 | 20p12 | bone morphogenetic protein 2 |
| PLCB1 | 8112.912 | 20p12 | phospholipase C, beta 1 (phosphoinositide-specific) |
| PLCB4 | 9288.447 | 20p12 | phospholipase C, beta 4 |
| PAK7 | 9518.037 | 20p12 | p21 protein (Cdc42/Rac)-activated kinase 7 |
| MKKS | 10385.428 | 20p12 | McKusick-Kaufman syndrome |
| TASP1 | 13370.036 | 20p12 | taspase, threonine aspartase, 1 |
| RRBP1 | 17594.323 | 20p12 | ribosome binding protein 1 |
| SNAP25 | 10199.477 | 20p12-p11.2 | synaptosomal-associated protein, 25 kDa |
| SPTLC3 | 12989.627 | 20p12.1 | serine palmitoyltransferase, long chain base subunit 3 |
| ESF1 | 13694.969 | 20p12.1 | ESF1, nucleolar pre-rRNA processing protein, homolog (S. cerevisiae) |
| MACROD2 | 15177.504 | 20p12.1 | MACRO domain containing 2 |
| DSTN | 17550.599 | 20p12.1 | destrin (actin depolymerizing factor) |
| JAG1 | 10618.332 | 20p12.1-p11.23 | jagged 1 |
| OTOR | 16728.998 | 20p12.1-p11.23 | otoraplin |
| MCM8 | 5931.298 | 20p12.3 | minichromosome maintenance complex component 8 |
| FERMT1 | 6055.492 | 20p12.3 | fermitin family member 1 |
| CASC20 | 6407.379 | 20p12.3 | cancer susceptibility candidate 20 (non-protein coding) |
| NRSN2 | 327.37 | 20p13 | neurensin 2 |
| RBCK1 | 388.709 | 20p13 | RanBP-type and C3HC4-type zinc finger containing 1 |
| CSNK2A1 | 463.338 | 20p13 | casein kinase 2, alpha 1 polypeptide |
| TCF15 | 584.637 | 20p13 | transcription factor 15 (basic helix-loop-helix) |
| SRXN1 | 627.268 | 20p13 | sulfiredoxin 1 |
| SLC52A3 | 740.724 | 20p13 | solute carrier family 52 (riboflavin transporter), member 3 |
| ANGPT4 | 853.297 | 20p13 | angiopoietin 4 |
| RAD21L1 | 1206.764 | 20p13 | RAD21-like 1 (S. pombe) |
| FKBP1A | 1349.621 | 20p13 | FK506 binding protein 1A, 12 kDa |
| SIRPB2 | 1455.236 | 20p13 | signal-regulatory protein beta 2 |
| SIRPB1 | 1545.029 | 20p13 | signal-regulatory protein beta 1 |
| SIRPG | 1609.798 | 20p13 | signal-regulatory protein gamma |
| SIRPA | 1874.813 | 20p13 | signal-regulatory protein alpha |
| PDYN | 1959.402 | 20p13 | prodynorphin |
| STK35 | 2082.528 | 20p13 | serine/threonine kinase 35 |
| TMC2 | 2517.253 | 20p13 | transmembrane channel-like 2 |
| IDH3B | 2639.661 | 20p13 | isocitrate dehydrogenase 3 (NAD+) beta |
| PTPRA | 2844.841 | 20p13 | protein tyrosine phosphatase, receptor type, A |
| GNRH2 | 3024.268 | 20p13 | gonadotropin-releasing hormone 2 |
| OXT | 3052.266 | 20p13 | oxytocin/neurophysin I prepropeptide |
| AVP | 3063.202 | 20p13 | arginine vasopressin |
| UBOX5 | 3088.219 | 20p13 | U-box domain containing 5 |
| LZTS3 | 3143.263 | 20p13 | leucine zipper, putative tumor suppressor family member 3 |
| ADAM33 | 3648.62 | 20p13 | ADAM metallopeptidase domain 33 |
| SIGLEC1 | 3667.617 | 20p13 | sialic acid binding Ig-like lectin 1, sialoadhesin |
| HSPA12B | 3713.317 | 20p13 | heat shock 70 kD protein 12B |
| CENPB | 3764.498 | 20p13 | centromere protein B, 80 kDa |
| CDC25B | 3767.419 | 20p13 | cell division cycle 25B |
| AP5S1 | 3801.171 | 20p13 | adaptor-related protein complex 5, sigma 1 subunit |
| MAVS | 3827.446 | 20p13 | mitochondrial antiviral signaling protein |
| RNF24 | 3912.069 | 20p13 | ring finger protein 24 |
| SMOX | 4129.426 | 20p13 | spermine oxidase |
| PRNP | 4666.797 | 20p13 | prion protein |
| RASSF2 | 4760.67 | 20p13 | Ras association (RalGDS/AF-6) domain family member 2 |
| SLC23A2 | 4833.002 | 20p13 | solute carrier family 23 (ascorbic acid transporter), member 2 |
| CDS2 | 5107.407 | 20p13 | CDP-diacylglycerol synthase (phosphatidate cytidylyltransferase) 2 |
| TRIB3 | 361.308 | 20p13-p12.2 | tribbles pseudokinase 3 |
| CRLS1 | 5987.898 | 20p13-p12.3 | cardiolipin synthase 1 |
| PRND | 4702.5 | 20pter-p12 | prion protein 2 (dublet) |
| PCNA | 5095.599 | 20pter-p12 | proliferating cell nuclear antigen |
| CHGB | 5891.974 | 20pter-p12 | chromogranin B (secretogranin 1) |
| HRH3 | 60790.017 | 20pter-p12.1 | histamine receptor H3 |
| ERGIC3 | 34129.778 | 20pter-q12 | ERGIC and golgi 3 |
| SAMHD1 | 35520.227 | 20pter-q12 | SAM domain and HD domain 1 |
| ID1 | 30193.086 | 20q11 | inhibitor of DNA binding 1, dominant negative helix-loop-helix protein |
| TTLL9 | 30458.505 | 20q11 | tubulin tyrosine ligase-like family, member 9 |
| POFUT1 | 30795.696 | 20q11 | protein O-fucosyltransferase 1 |
| ASXL1 | 30946.147 | 20q11 | additional sex combs like transcriptional regulator 1 |
| COMMD7 | 31290.493 | 20q11 | COMM domain containing 7 |
| CBFA2T2 | 32077.928 | 20q11 | core-binding factor, runt domain, alpha subunit 2; translocated to, 2 |
| E2F1 | 32263.292 | 20q11 | E2F transcription factor 1 |
| NCOA6 | 33302.578 | 20q11 | nuclear receptor coactivator 6 |
| HCK | 30639.991 | 20q11-q12 | HCK proto-oncogene, Src family tyrosine kinase |
| FOXS1 | 30432.103 | 20q11.1-q11.2 | forkheadbox S1 |
| MAPRE1 | 31407.699 | 20q11.1-q11.3 | microtubule-associated protein, RP/EB family, member 1 |
| MAFB | 39314.488 | 20q11.1-q13.1 | v-maf avian musculoaponeurotic fibrosarcoma oncogene homolog B |
| TGM3 | 2276.613 | 20q11.2 | transglutaminase 3 |
| TPX2 | 30326.904 | 20q11.2 | TPX2, microtubule-associated |
| DNMT3B | 31350.191 | 20q11.2 | DNA (cytosine-5-)-methyltransferase 3 beta |
| SNTA1 | 31995.763 | 20q11.2 | syntrophin, alpha 1 |
| EIF2S2 | 32676.115 | 20q11.2 | eukaryotic translation initiation factor 2, subunit 2 beta, 38 kDa |
| GSS | 33516.236 | 20q11.2 | glutathione synthetase |
| PROCR | 33759.74 | 20q11.2 | protein C receptor, endothelial |
| MMP24 | 33814.539 | 20q11.2 | matrix metallopeptidase 24 (membrane-inserted) |
| EIF6 | 33866.709 | 20q11.2 | eukaryotic translation initiation factor 6 |
| GDF5 | 34021.145 | 20q11.2 | growth differentiation factor 5 |
| SPAG4 | 34203.809 | 20q11.2 | sperm associated antigen 4 |
| GHRH | 35879.49 | 20q11.2 | growth hormone releasing hormone |
| ASIP | 32848.171 | 20q11.2-q12 | agouti signaling protein |
| NNAT | 36149.607 | 20q11.2-q12 | neuronatin |
| STK4 | 43595.12 | 20q11.2-q13.2 | serine/threonine kinase 4 |
| MLLT10P1 | 29637.584 | 20q11.21 | myeloid/lymphoid or mixed-lineage leukemia (trithorax homolog, |
| | | | Drosophila); translocated to, 10 pseudogene 1 |
| HM13 | 30102.213 | 20q11.21 | histocompatibility (minor) 13 |
| COX4I2 | 30225.691 | 20q11.21 | cytochrome c oxidase subunit IV isoform 2 (lung) |
| BCL2L1 | 30252.261 | 20q11.21 | BCL2-like 1 |
| DUSP15 | 30448.87 | 20q11.21 | dual specificity phosphatase 15 |
| PDRG1 | 30532.758 | 20q11.21 | p53 and DNA-damage regulated 1 |
| TM9SF4 | 30697.309 | 20q11.21 | transmembrane 9 superfamily protein member 4 |
| PLAGL2 | 30780.307 | 20q11.21 | pleiomorphic adenoma gene-like 2 |
| KIF3B | 30865.454 | 20q11.21 | kinesin family member 3B |
| NOL4L | 31030.862 | 20q11.21 | nucleolar protein 4-like |
| BPIFB2 | 31595.384 | 20q11.21 | BPI fold containing family B, member 2 |
| BPIFA4P | 31781.411 | 20q11.21 | BPI fold containing family A, member 4, pseudogene |
| BPIFA1 | 31823.802 | 20q11.21 | BPI fold containing family A, member 1 |
| BPIFB1 | 31870.941 | 20q11.21 | BPI fold containing family B, member 1 |
| CDK5RAP1 | 31946.645 | 20q11.21 | CDK5 regulatory subunit associated protein 1 |
| DYNLRB1 | 33104.189 | 20q11.21 | dynein, light chain, roadblock-type 1 |
| RALY | 32581.458 | 20q11.21-q11.23 | RALY heterogeneous nuclear ribonucleoprotein |
| NDRG3 | 35280.169 | 20q11.21-q11.23 | NDRG family member 3 |
| RPRD1B | 36661.948 | 20q11.21-q12 | regulation of nuclear pre-mRNA domain containing 1B |
| AHCY | 32868.071 | 20q11.22 | adenosylhomocysteinase |
| ITCH | 32951.041 | 20q11.22 | itchy E3 ubiquitin protein ligase |
| MAP1LC3A | 33146.501 | 20q11.22 | microtubule-associated protein 1 light chain 3 alpha |
| PIGU | 33148.346 | 20q11.22 | phosphatidylinositol glycan anchor biosynthesis, class U |
| TP53INP2 | 33292.148 | 20q11.22 | tumor protein p53 inducible nuclear protein 2 |
| GGT7 | 33432.523 | 20q11.22 | gamma-glutamyltransferase 7 |
| ACSS2 | 33464.328 | 20q11.22 | acyl-CoA synthetase short-chain family member 2 |
| MIR499A | 33578.179 | 20q11.22 | microRNA 499a |
| UQCC1 | 33890.369 | 20q11.22 | ubiquinol-cytochrome c reductase complex assembly factor 1 |
| CEP250 | 34043.223 | 20q11.22 | centrosomal protein 250 kDa |
| CPNE1 | 34213.953 | 20q11.22 | copine I |
| ROMO1 | 34287.232 | 20q11.22 | reactive oxygen species modulator 1 |
| RBM39 | 34291.531 | 20q11.22 | RNA binding motif protein 39 |
| PHF20 | 34359.923 | 20q11.22-q11.23 | PHD finger protein 20 |
| DHX35 | 37590.981 | 20q11.22-q12 | DEAH (Asp-Glu-Ala-His) box polypeptide 35 |
| TRPC4AP | 33590.207 | 20q11.23 | transient receptor potential cation channel, subfamily C, member 4 |
| | | | associated protein |
| DLGAP4 | 35089.818 | 20q11.23 | discs, large (Drosophila) homolog-associated protein 4 |
| MYL9 | 35169.887 | 20q11.23 | myosin, light chain 9, regulatory |
| TGIF2 | 35201.876 | 20q11.23 | TGFB-induced factor homeobox 2 |
| C20orf24 | 35234.137 | 20q11.23 | chromosome 20 open reading frame 24 |
| SLA2 | 35240.924 | 20q11.23 | Src-like-adaptor 2 |
| RBL1 | 35624.755 | 20q11.23 | retinoblastoma-like 1 |
| BLCAP | 36145.819 | 20q11.23 | bladder cancer associated protein |
| BPI | 36932.552 | 20q11.23 | bactericidal/permeability-increasing protein |
| LBP | 36974.814 | 20q11.23 | lipopolysaccharide binding protein |
| PPP1R16B | 37434.348 | 20q11.23 | protein phosphatase 1, regulatory subunit 16B |
| CTNNBL1 | 36322.357 | 20q11.23-q12 | catenin, beta like 1 |
| TGM2 | 36756.864 | 20q12 | transglutaminase 2 |
| ACTR5 | 37377.097 | 20q12 | ARP5 actin-related protein 5 homolog (yeast) |
| EMILIN3 | 39988.606 | 20q12 | elastin microfibril interfacer 3 |
| CHD6 | 40030.743 | 20q12 | chromodomain helicase DNA binding protein 6 |
| SDC4 | 43953.929 | 20q12 | syndecan 4 |
| NCOA3 | 46130.601 | 20q12 | nuclear receptor coactivator 3 |
| SRC | 35973.088 | 20q12-q13 | SRC proto-oncogene, non-receptor tyrosine kinase |
| PTPRT | 40701.392 | 20q12-q13 | protein tyrosine phosphatase, receptor type, T |
| MMP9 | 44637.547 | 20q12-q13 | matrix metallopeptidase 9 (gelatinase B, 92 kDa gelatinase, 92 kDa type |
| | | | IV collagenase) |
| RPN2 | 35807.456 | 20q12-q13.1 | ribophorin II |
| TOP1 | 39657.462 | 20q12-q13.1 | topoisomerase (DNA) I |
| PLCG1 | 39766.161 | 20q12-q13.1 | phospholipase C, gamma 1 |
| SRSF6 | 42086.504 | 20q12-q13.1 | serine/arginine-rich splicing factor 6 |
| TOMM34 | 43570.771 | 20q12-q13.1 | translocase of outer mitochondrial membrane 34 |
| SEMG2 | 43850.01 | 20q12-q13.1 | semenogelin II |
| PIGT | 44044.707 | 20q12-q13.12 | phosphatidylinositol glycan anchor biosynthesis, class T |
| NCOA5 | 44689.626 | 20q12-q13.12 | nuclear receptor coactivator 5 |
| SEMG1 | 43835.638 | 20q12-q13.2 | semenogelin I |
| CD40 | 44746.906 | 20q12-q13.2 | CD40 molecule, TNF receptor superfamily member 5 |
| CSE1L | 47662.783 | 20q13 | CSE1 chromosome segregation 1 -like (yeast) |
| PTGIS | 48120.411 | 20q13 | prostaglandin 12 (prostacyclin) synthase |
| BCAS4 | 49411.431 | 20q13 | breast carcinoma amplified sequence 4 |
| CYP24A1 | 52769.988 | 20q13 | cytochrome P450, family 24, subfamily A, polypeptide 1 |
| AURKA | 54944.445 | 20q13 | aurora kinase A |
| BMP7 | 55743.809 | 20q13 | bone morphogenetic protein 7 |
| RAB22A | 56884.771 | 20q13 | RAB22A, member RAS oncogene family |
| VAPB | 56964.175 | 20q13 | VAMP (vesicle-associated membrane protein)-associated protein B and |
| | | | C |
| NELFCD | 57556.263 | 20q13 | negative elongation factor complex member C/D |
| NTSR1 | 61340.189 | 20q13 | neurotensin receptor 1 (high affinity) |
| MYBL2 | 42295.659 | 20q13.1 | v-myb avian myeloblastosis viral oncogene homolog-like 2 |
| YWHAB | 43514.24 | 20q13.1 | tyrosine 3-monooxygenase/tryptophan 5-monooxygenase activation |
| | | | protein, beta |
| CDH22 | 44802.372 | 20q13.1 | cadherin 22, type 2 |
| EYA2 | 45523.263 | 20q13.1 | EYA transcriptional coactivator and phosphatase 2 |
| STAU1 | 47729.876 | 20q13.1 | staufen double-stranded RNA binding protein 1 |
| CEBPB | 48807.12 | 20q13.1 | CCAAT/enhancer binding protein (C/EBP), beta |
| DPM1 | 49551.405 | 20q13.1 | dolichyl-phosphate mannosytransferase polypeptide 1, catalytic subunit |
| B4GALT5 | 48249.483 | 20q13.1-q13.2 | UDP-Gal:betaGlcNAc beta 1,4- galactosyltransferase, polypeptide 5 |
| PTPN1 | 49126.858 | 20q13.1-q13.2 | protein tyrosine phosphatase, non-receptor type 1 |
| WFDC5 | 43738.093 | 20q13.11 | WAP four-disulfide core domain 5 |
| ZFP64 | 50767.817 | 20q13.11-q13.13 | ZFP64 zinc finger protein |
| L3MBTL1 | 42143.076 | 20q13.12 | l(3)mbt-like 1 (Drosophila) |
| TOX2 | 42544.782 | 20q13.12 | TOX high mobility group box family member 2 |
| HNF4A | 43029.896 | 20q13.12 | hepatocyte nuclear factor 4, alpha |
| SERINC3 | 43127.901 | 20q13.12 | serine incorporator 3 |
| ADA | 43248.163 | 20q13.12 | adenosine deaminase |
| WISP2 | 43343.885 | 20q13.12 | WNT1 inducible signaling pathway protein 2 |
| PI3 | 43803.54 | 20q13.12 | peptidase inhibitor 3, skin-derived |
| SLPI | 43880.88 | 20q13.12 | secretory leukocyte peptidase inhibitor |
| WFDC2 | 44098.394 | 20q13.12 | WAP four-disulfide core domain 2 |
| EPPIN | 44169.265 | 20q13.12 | epididymal peptidase inhibitor |
| UBE2C | 44441.215 | 20q13.12 | ubiquitin-conjugating enzyme E2C |
| ACOT8 | 44470.36 | 20q13.12 | acyl-CoA thioesterase 8 |
| NEURL2 | 44517.111 | 20q13.12 | neuralized E3 ubiquitin protein ligase 2 |
| CTSA | 44519.591 | 20q13.12 | cathepsin A |
| PLTP | 44527.259 | 20q13.12 | phospholipid transfer protein |
| ZNF335 | 44577.292 | 20q13.12 | zinc finger protein 335 |
| SLC12A5 | 44650.329 | 20q13.12 | solute carrier family 12 (potassium/chloride transporter), member 5 |
| SLC35C2 | 44978.167 | 20q13.12 | solute carrier family 35 (GDP-fucose transporter), member C2 |
| OCSTAMP | 45169.67 | 20q13.12 | osteoclast stimulatory transmembrane protein |
| SLC2A10 | 45338.279 | 20q13.12 | solute carrier family 2 (facilitated glucose transporter), member 10 |
| ZMYND8 | 45926.87 | 20q13.12 | zinc finger, MYND-type containing 8 |
| SULF2 | 46286.15 | 20q13.12-q13.13 | sulfatase 2 |
| PREX1 | 47240.793 | 20q13.13 | phosphatidylinositol-3,4,5-trisphosphate-dependent Rac exchange factor |
| | | | 1 |
| ARFGEF2 | 47538.275 | 20q13.13 | ADP-ribosylation factor guanine nucleotide-exchange factor 2 (brefeldin |
| | | | A-inhibited) |
| DDX27 | 47835.832 | 20q13.13 | DEAD (Asp-Glu-Ala-Asp) box polypeptide 27 |
| ZNFX1 | 47862.439 | 20q13.13 | zinc finger, NFX1-type containing 1 |
| ZFAS1 | 47894.715 | 20q13.13 | ZNFX1 antisense RNA 1 |
| SLC9A8 | 48429.25 | 20q13.13 | solute carrier family 9, subfamily A (NHE8, cation proton antiporter 8), |
| | | | member 8 |
| SPATA2 | 48519.929 | 20q13.13 | spermatogenesis associated 2 |
| TMEM189- | 48697.661 | 20q13.13 | TMEM189-UBE2V1 readthrough |
| UBE2V1 | | | |
| FAM65C | 49202.645 | 20q13.13 | family with sequence similarity 65, member C |
| ADNP | 49505.455 | 20q13.13 | activity-dependent neuroprotector homeobox |
| SGK2 | 42193.755 | 20q13.2 | serum/glucocorticoid regulated kinase 2 |
| TP53RK | 45313.004 | 20q13.2 | TP53 regulating kinase |
| SNAI1 | 48599.513 | 20q13.2 | snail family zinc finger 1 |
| UBE2V1 | 48697.661 | 20q13.2 | ubiquitin-conjugating enzyme E2 variant 1 |
| NFATC2 | 50003.494 | 20q13.2 | nuclear factor of activated T-cells, cytoplasmic, calcineurin-dependent 2 |
| ATP9A | 50213.314 | 20q13.2 | ATPase, class II, type 9A |
| SALL4 | 50400.583 | 20q13.2 | spalt-like transcription factor 4 |
| TSHZ2 | 51801.822 | 20q13.2 | teashirt zinc finger homeobox 2 |
| ZNF217 | 52183.61 | 20q13.2 | zinc finger protein 217 |
| BCAS1 | 52560.079 | 20q13.2 | breast carcinoma amplified sequence 1 |
| PFDN4 | 52824.502 | 20q13.2 | prefoldin subunit 4 |
| DOK5 | 53092.011 | 20q13.2 | docking protein 5 |
| TFAP2C | 55204.358 | 20q13.2 | transcription factor AP-2 gamma (activating enhancer binding protein 2 |
| | | | gamma) |
| GNAS | 57466.426 | 20q13.2-q13.3 | GNAS complex locus |
| EDN3 | 57875.499 | 20q13.2-q13.3 | endothelin 3 |
| LAMA5 | 60884.116 | 20q13.2-q13.3 | laminin, alpha 5 |
| TPD52L2 | 62496.581 | 20q13.2-q13.3 | tumor protein D52-like 2 |
| ATP5E | 57603.733 | 20q13.3 | ATP synthase, H+ transporting, mitochondrial F1 complex, epsilon |
| | | | subunit |
| PPP1R3D | 58511.887 | 20q13.3 | protein phosphatase 1, regulatory subunit 3D |
| CDH4 | 60074.477 | 20q13.3 | cadherin 4, type 1, R-cadherin (retinal) |
| SS18L1 | 60718.822 | 20q13.3 | synovial sarcoma translocation gene on chromosome 18-like 1 |
| OGFR | 61436.177 | 20q13.3 | opioid growth factor receptor |
| BIRC7 | 61867.235 | 20q13.3 | baculoviral IAP repeat containing 7 |
| EEF1A2 | 62119.365 | 20q13.3 | eukaryotic translation elongation factor 1 alpha 2 |
| PTK6 | 62159.776 | 20q13.3 | protein tyrosine kinase 6 |
| RTEL1 | 62289.163 | 20q13.3 | regulator of telomere elongation helicase 1 |
| ARFRP1 | 62329.995 | 20q13.3 | ADP-ribosylation factor related protein 1 |
| ZGPAT | 62338.794 | 20q13.3 | zinc finger, CCCH-type with G patch domain |
| MYLK2 | 30407.178 | 20q13.31 | myosin light chain kinase 2 |
| CSTF1 | 54967.427 | 20q13.31 | cleavage stimulation factor, 3′ pre-RNA, subunit 1, 50 kDa |
| CASS4 | 54987.314 | 20q13.31 | Cas scaffolding protein family member 4 |
| RAE1 | 55926.618 | 20q13.31 | ribonucleic acid export 1 |
| RBM38 | 55966.454 | 20q13.31 | RNA binding motif protein 38 |
| CTCFL | 56072.224 | 20q13.31 | CCCTC-binding factor (zinc finger protein)-like |
| PCK1 | 56136.137 | 20q13.31 | phosphoenolpyruvate carboxykinase 1 (soluble) |
| ZBP1 | 56178.902 | 20q13.31 | Z-DNA binding protein 1 |
| PMEPA1 | 56223.448 | 20q13.31-q13.33 | prostate transmembrane protein, androgen induced 1 |
| C20orf85 | 56725.983 | 20q13.32 | chromosome 20 open reading frame 85 |
| PPP4R1L | 56812.975 | 20q13.32 | protein phosphatase 4, regulatory subunit 1 -like |
| APCDD1L | 57034.426 | 20q13.32 | adenomatosis polyposis coli down-regulated 1 -like |
| STX16 | 57226.309 | 20q13.32 | syntaxin 16 |
| MIR296 | 57392.67 | 20q13.32 | microRNA 296 |
| MIR298 | 57393.281 | 20q13.32 | microRNA 298 |
| CTSZ | 57570.242 | 20q13.32 | cathepsin Z |
| TUBB1 | 57594.309 | 20q13.32 | tubulin, beta 1 class VI |
| PHACTR3 | 58152.564 | 20q13.32-q13.33 | phosphatase and actin regulator 3 |
| TAF4 | 60549.854 | 20q13.33 | TAF4 RNA polymerase II, TATA box binding protein (TBP)-associated |
| | | | factor, 135 kDa |
| PSMA7 | 60711.783 | 20q13.33 | proteasome (prosome, macropain) subunit, alpha type, 7 |
| ADRM1 | 60877.952 | 20q13.33 | adhesion regulating molecule 1 |
| GATA5 | 61038.553 | 20q13.33 | GATA binding protein 5 |
| MIR1-1 | 61151.513 | 20q13.33 | microRNA 1-1 |
| MIR133A2 | 61162.119 | 20q13.33 | microRNA 133a-2 |
| MRGBP | 61427.805 | 20q13.33 | MRG/MORF4L binding protein |
| DIDO1 | 61509.09 | 20q13.33 | death inducer-obliterator 1 |
| MIR124-3 | 61809.852 | 20q13.33 | microRNA 124-3 |
| SRMS | 62171.277 | 20q13.33 | src-related kinase lacking C-terminal regulatory tyrosine and N-terminal |
| | | | myristylation sites |
| TNFRSF6B | 62328.004 | 20q13.33 | tumor necrosis factor receptor superfamily, member 6b, decoy |
| DNAJC5 | 62526.455 | 20q13.33 | DnaJ (Hsp40) homolog, subfamily C, member 5 |
| UCKL1 | 62571.182 | 20q13.33 | uridine-cytidine kinase 1 -like 1 |
| PRPF6 | 62612.431 | 20q13.33 | pre-mRNA processing factor 6 |
| SOX18 | 62679.079 | 20q13.33 | SRY (sex determining region Y)-box 18 |
| RGS19 | 62704.535 | 20q13.33 | regulator of G-protein signaling 19 |
| MYT1 | 62795.827 | 20q13.33 | myelin transcription factor 1 |

* Information obtained from http://atlasgeneticsoncology.org/Indexbychrom/idxg_20.html (last accessed on Sep. 11, 2014)

1. A method for identifying an abnormal sample of cells comprising:
a) hybridizing a set of chromosomal probes to the sample, wherein the set comprises probes to 3q26, 5p15, CEP7, and 20q13;
b) evaluating cells of the sample to detect and quantify the presence of each probe in the set;
c) categorizing the evaluated cells of the sample as normal or abnormal, wherein the normal cells contain exactly two copies of each probe in the set and the abnormal cells do not contain exactly two copies of each probe in the set;
d) calculating the percentage of the abnormal cells in the evaluated cells of the sample; and
e) identifying the sample of cells as abnormal if the percentage of abnormal cells in the evaluated cells is greater than or equal to a cut-off value of 0.3%.

2. The method of claim 1, wherein the sample of cells is a sample of cervical, vaginal, or anal cells.

3. The method of claim 2, wherein the abnormal cells are selected from the group consisting of: cells having a single gain, cells having multiple gains, tetra-ploid cells, and combinations thereof.

4. The method of claim 3, wherein a minimum of 1,000 cells in the sample are evaluated.

5. The method of claim 4, wherein the sample of cells is classified as abnormal if:
i. the percentage of cells having a single gain is ≧0.3%;
ii. the percentage of cells having multiple gains is ≧0.7%; or
iii. the percentage of tetra-ploid cells is ≧0.8%.

6. The method of claim 4, wherein the sample of cells is classified as abnormal if:
i. the percentage of cells having a single gain is ≧0.7%;
ii. the percentage of cells having multiple gains is ≧1.0%; or
iii. the percentage of tetra-ploid cells is ≧1.1%.

7. The method of claim 4, wherein the sample of cells is classified as abnormal if:
i. the percentage of cells having a single gain is ≧1.2%;
ii. the percentage of cells having multiple gains is ≧0.7%; or
iii. the percentage of tetra-ploid cells is ≧0.8%.

8. The method of claim 4, wherein the sample of cells is classified as abnormal if:
i. the percentage of cells having a gain in 3q26 is ≧1.3%;
ii. the percentage of cells having a gain in 5p15 is ≧1.2%;
iii. the percentage of cells having a gain in CEP7 is ≧1.0%;
iv. the percentage of cells having a gain in 20q13 is ≧1.0%;
v. the percentage of cells having multiple gains is ≧1.3%; or
vi. the percentage of tetra-ploid cells is ≧1.5%.

9. The method of claim 4, wherein the sample of cells is classified as abnormal if:
i. the percentage of cells having a gain in 3q26 is ≧2.2%;
ii. the percentage of cells having a gain in 5p15 is ≧3.2%;
iii. the percentage of cells having a gain in CEP7 is ≧1.6%;
iv. the percentage of cells having a gain in 20q13 is ≧0.9%.
v. the percentage of cells having multiple gains is ≧1.0%; or
vi. the percentage of tetra-ploid cells is ≧1.2%.

10. The method of claim 1, wherein the steps of the method are performed manually.

11. The method of claim 1, wherein the steps of the method are performed by an automated system.

12. The method of claim 11, further comprising the step of verifying steps (b)-(e) manually.

13. The method of claim 11, further comprising the step of verifying steps (b)-(e) manually anytime an abnormal cell having a multiple gains is detected by the automated system.

14. A method for detecting an abnormal sample of cervical cells comprising:
a) hybridizing a first nucleic acid probe to a target nucleic acid sequence on chromosome 3q of the cervical cells to form a first hybridization complex;
b) hybridizing a second nucleic acid probe to a target nucleic acid on chromosome 5p of the cervical cells to form a second hybridization complex;
c) hybridizing a third nucleic acid probe to a target nucleic acid on chromosome 20q of the cervical cells to form a third hybridization complex;
d) hybridizing a fourth nucleic acid probe to centromere of chromosome 7 (CEN7) to form a fourth hybridization complex;
e) evaluating cells within the sample to detect and quantify:
i. the formation of the first hybridization complex on chromosome 3q;
ii. the formation of the second hybridization complex on chromosome 5p;
iii. the formation of the third hybridization complex on 20q;
iv. the formation of the fourth hybridization complex on CEN7,
f) categorizing each cell within the evaluated cells as normal or abnormal, wherein
i. the normal cell contains exactly two copies of 3q, 5p, 20q, and CEN7; and
ii. the abnormal cell contains more than two copies of 3q, 5p, 20q, CEN7, or a combination thereof;
g) calculating the percentage of abnormal cells present in the evaluated cells of the sample; and
h) classifying the sample of cervical cells as abnormal if the percentage of abnormal cells in the evaluated cells is greater than or equal to a cut-off value of 0.3%.

15. The method of claim 14, wherein the abnormal cells are selected from the group consisting of: cells having a single gain, cells having multiple gains, tetra-ploid cells, and combinations thereof.

16. The method of claim 14, wherein a minimum of 1,000 cells in the sample are evaluated.

17. The method of claim 14, wherein the sample of cells is classified as abnormal if:
i. the percentage of cells having a gain in 3q26 is ≧1.3%;
ii. the percentage of cells having a gain in 5p15 is ≧1.2%;
iii. the percentage of cells having a gain in CEP7 is ≧1.0%;
iv. the percentage of cells having a gain in 20q13 is ≧1.0%;
v. the percentage of cells having multiple gains is ≧1.3%; or
vi. the percentage of tetra-ploid cells is ≧1.5%.

18. The method of claim 14, wherein the sample of cells is classified as abnormal if:
i. the percentage of cells having a gain in 3q26 is ≧2.2%;
ii. the percentage of cells having a gain in 5p15 is ≧3.2%;
iii. the percentage of cells having a gain in CEP7 is ≧1.6%;
iv. the percentage of cells having a gain in 20q13 is ≧0.9%.
v. the percentage of cells having multiple gains is ≧1.0%; or
vi. the percentage of tetra-ploid cells is ≧1.2%.

19. The method of claim 14, wherein the steps of the method are performed by an automated system.

20. A method for detecting an abnormal sample of cervical cells comprising:
a) hybridizing a first nucleic acid probe to a target nucleic acid sequence on 3q26 of the cervical cells to form a first hybridization complex;
b) hybridizing a second nucleic acid probe to a target nucleic acid on 5p15 of the cervical cells to form a second hybridization complex;
c) hybridizing a third nucleic acid probe to a target nucleic acid on 20q13 of the cervical cells to form a third hybridization complex;
d) hybridizing a fourth nucleic acid probe to centromere of chromosome 7 (CEN7) to form a fourth hybridization complex;
e) evaluating at least 1,000 cells within the sample to detect and quantify:
i. the formation of the first hybridization complex on chromosome 3q26;
ii. the formation of the second hybridization complex on chromosome 5p15;
iii. the formation of the third hybridization complex on 20q13;
iv. the formation of the fourth hybridization complex on CEN7,
f) categorizing each cell within the evaluated cells as normal or abnormal, wherein
i. the normal cell contains exactly two copies of 3q26, 5p15, 20q13, and CEN7; and
ii. the abnormal cell is selected from the group consisting of: a cell having a single gain, a cell having multiple gains, a tetra-ploid cell, and combinations thereof;
g) calculating the percentage of abnormal cells present in the evaluated cells of the sample; wherein the steps of (a)-(g) are performed manually or by an automated system, the method further comprising the step of
h) classifying the entire sample of cervical cells as abnormal if, the following percentages of abnormal cells are observed when the steps of (a)-(g) are performed manually:
i. cells having a gain in 3q26 is ≧1.3%;
ii. cells having a gain in 5p15 is ≧1.2%;
iii. cells having a gain in CEP7 is ≧1.0%;
iv. cells having a gain in 20q13 is ≧1.0%.
v. cells having multiple gains is ≧1.3%; or
vi. tetra-ploid cells is ≧1.5%;
or
i) classifying the entire sample of cervical cells as abnormal if, the following percentages of abnormal cells are observed when the steps of (a)-(g) are performed by an automated system:
i. cells having a gain in 3q26 is ≧2.2%;
ii. cells having a gain in 5p15 is ≧3.2%;
iii. cells having a gain in CEP7 is ≧1.6%;
iv. cells having a gain in 20q13 is ≧0.9%.
v. cells having multiple gains is ≧1.0%; or
vi. tetra-ploid cells is ≧1.2%.
